(19) United States
(12) Patent Application Publication
(10) Pub. No.: US 2026/0098019 A1
(43) Pub. Date: Apr. 9, 2026

(54) SYNTHESIS OF NSAID CONJUGATES AS ANTI-INFLAMMATORY AGENTS USING THE MOLECULAR HYBRIDIZATION

(71) Applicant: AUGUSTA UNIVERSITY RESEARCH INSTITUTE, INC., Augusta, GA (US)

(72) Inventor: Siva PANDA, Augusta, GA (US)

(21) Appl. No.: 19/112,976

(22) PCT Filed: Oct. 9, 2023
(86) PCT No.: PCT/US2023/076359

§ 371 Date: Mar. 19, 2025

Related U.S. Application Data
(60) Provisional application No. 63/414,197, filed on Oct. 7, 2022.

(51) Int. Cl. C07D 249/06; A61K 31/4192; A61K 45/06; A61P 29/00; C07D 403/12

(52) U.S. Cl. CPC C07D 249/06; A61K 31/4192; A61K 45/06; A61P 29/00; C07D 403/12

(57) ABSTRACT

Described are synthetic compounds having anti-inflammatory properties and optionally analgesic properties. These compounds are derivatives of FDA-approved anti-inflammatory, such as ibuprofen and indomethacin, and can be formed using reactions under microwave irradiation. Generally, the compounds contains a moiety of the parent drug, a triazolyl heterocycle, and a substituted or unsubstituted aryl group. In some forms, the compounds show anti-inflammatory properties and optionally analgesic properties with similar or higher efficiencies compared with their respective parent drugs, with additional advantages including no or reduced adverse effects (e.g., without ulcerogenic liability in the gastric) and/or selective inhibition of COX-2 over COX-1. Pharmaceutical compositions suitable for the delivery of the compounds to a subject in need thereof are disclosed. The pharmaceutical formulation can be administered by oral administration, parenteral administration, inhalation, mucosal administration, or a combination thereof. Methods for preventing or treating an inflammatory disease or disorder in a subject are also disclosed.

### CROSS-REFERENCE TO RELATED APPLICATIONS

This application claims the benefit of and priority to U.S. Provisional Application No. 63/414,197 filed on Oct. 7, 2022, the contents of which are specifically incorporated herein in their entireties.

### FIELD OF THE INVENTION

The disclosed invention is generally in the field of synthetic compounds having anti-inflammatory properties, and methods of use thereof.

### BACKGROUND OF THE INVENTION

Non-steroidal anti-inflammatory drugs (NSAIDs) are used as the first-line drugs against inflammation, pain, and fever. The non-selective inhibition nature of traditional NSAIDs against the cyclooxygenase (COX) isoenzymes (COX-1 and COX-2) restricts their uses due to the associated gastrointestinal and ulcerogenic side effects. The COX enzymes are responsible for the metabolic conversion of arachidonic acid to prostanoids including prostaglandin H₂. COX exists in three isoforms: cyclooxygenase-1, 2, and 3 (COX-1, COX-2, and COX-3). COX-1 and COX-2 are of primary interest, as they are involved in physiological as well as pathological processes. COX-1 is responsible for the prostaglandin mediated functions of the gastrointestinal and cardiovascular systems. Under normal conditions, COX-2 is present at a low level. COX-2 is expressed in response to pro-inflammatory and pathogenic stimuli. In addition. COX-2 plays a critical role not only in inflammation, but also in various pathologies that include cancer, neurodegenerative diseases, and multidrug resistance.

The development of new drug candidates with selective inhibitory effect against COX-2 over COX-1 is challenging as both isoforms possess similar cellular expression locations and more than 60% sequence homology. Further, known COX-2 inhibitor drugs (e.g., celecoxib and rofecoxib) have serious drawbacks, especially in the cardiovascular system.

There remains a need to develop non-steroidal anti-inflammatory drugs, with no or reduced side effects, such as ulcers or erosions to the stomach.

Therefore, it is an object of the present invention to provide synthetic non-steroidal compounds that process anti-inflammatory properties, with no or reduced side effects, such as ulcers or erosions to the gastric.

It is a further object of the present invention to provide methods of making synthetic non-steroidal compounds that are non-steroidal and process anti-inflammatory properties, with no or reduced side effects, such as ulcers or erosions to the gastric.

It is a further object of the present invention to provide methods for using synthetic non-steroidal compounds that are non-steroidal and process anti-inflammatory properties, with no or reduced side effects, such as ulcers or erosions to the gastric.

### SUMMARY OF THE INVENTION

Described herein are synthetic non-steroidal compounds with anti-inflammatory properties and/or peripheral and/or central analgesic properties. Pharmaceutical formulations containing the compounds and methods of making and using the compounds are also disclosed.

The disclosed compounds are derivatives of FDA-approved anti-inflammatory drugs (also referred to herein as “parent drugs”), such as ibuprofen and indomethacin, and can be formed by conjugation of a substituted or unsubstituted aryl group to the parent, drug via azide-alkyne click chemistry (and thus the compounds are also referred to herein as “conjugates”). The conjugates contain a moiety of the parent drug that corresponds to the unreacted portion of the parent drug, a triazolyl heterocycle, and a substituted or unsubstituted aryl group. Optionally, the parent drug moiety and the triazolyl heterocycle are linked by a linker.

Generally, the conjugates show anti-inflammatory, antipyretic properties and/or analgesic properties with similar or higher potency and/or efficacy compared with their respective parent drugs, and should be suitable for use in the prevention or treatment of a variety of anti-inflammatory disease or disorders, and in providing analgesia to subjects in need thereof. The conjugates have additional advantages compared with the current NSAIDs, such as no or reduced adverse effects (e.g., without ulcerogenic effects).

In some forms, the compound can have the structure of

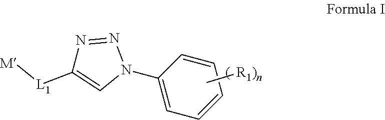

wherein: (i) R₁ is a halide, an unsubstituted alkyl, a substituted alkyl, an alkoxy, a nitro, a carbonyl, a hydroxyl, an amino, an amido, a cyano, a thio, an unsubstituted alkenyl, a substituted alkenyl, an unsubstituted alkynyl, a substituted alkynyl, an unsubstituted alkylaryl, a substituted alkylaryl, an unsubstituted aryl, or a substituted aryl;
(ii) n is an integer from 0 to 5, from 0 to 4, from 0 to 3, or 1 or 2, such as 1;
(iii) L₁ is

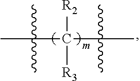

m is an integer from 0 to 6, R₂ and R₃ are independently H, an unsubstituted alkyl, or a substituted alkyl;
(iv) M′ is a parent drug moiety; and
(v) each substituent is independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a carbonyl, an alkoxy, a halogen, a hydroxyl, a phenoxy, a thiol, an alkylthio, a phenylthio, an arylthio, a cyano, an isocyano, a nitro, a carboxyl, an amino, an amido, an oxo, a silyl, a sulfinyl, a sulfonyl, a sulfonic acid, a phosphonium, a phosphanyl, a phosphoryl, or a phosphonyl.

In some forms, the parent drug is ibuprofen or indomethacin. In these forms, M′ of Formula I is a moiety of ibuprofen or indomethacin, having the structure of

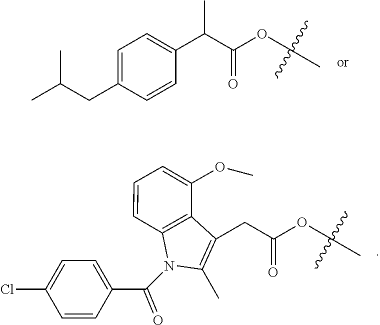 [SMILES: *OC(=O)C(C)c1ccc(CC(C)C)cc1 ; *OC(=O)Cc1c(C)n(C(=O)c2ccc(Cl)cc2)c2cccc(OC)c12]

Pharmaceutical compositions and pharmaceutical formulations, preferably, in a unit dosage form suitable for the delivery of the conjugates and their preparation are disclosed. Generally, the pharmaceutical composition or formulation contains one or more of the conjugates and a suitable pharmaceutically acceptable excipient. The one or more conjugates in the pharmaceutical compositions or formulations are in an effective amount for preventing or treating an inflammatory disease or disorder in a subject in need thereof. The pharmaceutical composition or formulation may further contain one or more additional active agents, such as one or more additional anti-inflammatory agents.

Methods of making the disclosed conjugates include exposing a reaction mixture to a microwave irradiation for a time period sufficient to form a product comprising the conjugate. The reaction mixture contains a first reactant which includes a portion of the parent compound, a second reactant, and a solvent. Typically, the microwave irradiation to which the reaction mixture is exposed has an energy suitable for heating the reaction mixture to a temperature effective to initiate the reaction and maintaining the reaction mixture at such a temperature for a suitable time period for the reaction to complete. For example, the microwave irradiation has an energy in a range from about 10 W to about 50 W, from about 10 W to about 40 W, from 10 W to about 30 W, or from 15 W to about 25 W, such as about 20 W. Using the methods described herein, conjugates can be produced at a yield of at least 60%, at least 65%, at least 70%, or at least 75%.

Methods of using the conjugates are disclosed. The conjugates are administered to a subject in need thereof, to prevent or treat an inflammatory disease or disorder, provide analgesia or as an antipyretic. The administration step can occur one or more times. The subject is typically a mammal, such as a human. In some forms of the method, the pharmaceutical formulation can be administered by oral administration, parenteral administration, inhalation, mucosal administration, topical or a combination thereof.

In some forms of the method, following a single administration of the pharmaceutical formulation following a dosage regimen, an effective amount of the conjugate is administered to the subject, and the amount is effective to prevent or reduce one or more symptoms of the inflammatory disease or disorder in the subject, as shown by one or more known clinical and/or biochemical measurements, such as inflammation-associated swelling of a body part (for example as indicated by the edema thickness of the body part); reduction in pain scale; reduced production of nitric oxide by macrophages and/or reduced release of one or more inflammatory cytokines from the macrophages, without any or with reduced ulcerogenic effects when compared to the parent compound. In some forms of the method, the conjugate(s) of the pharmaceutical formulation administered to the subject is/are in an effective amount to reduce or relief inflammation associated swelling of a body part and/or reduce or relief pain in the subject, with a similar or higher potency and/or efficacy compared with the treatment effect in a positive control, i.e., a subject having the same inflammatory disease or disorder as the subject treated with the pharmaceutical formulation but administered with a current NSAID, such as indomethacin, and/or compared to the parent compound of the conjugate.

### BRIEF DESCRIPTION OF THE DRAWINGS

FIG. 1 is a bar graph showing inhibition of edema thickness by exemplary conjugates.

FIG. 2A is a bar graph showing evaluation of NO production in RAW264.7 macrophages of Ibuprofen conjugates. 40 g/mL of ibuprofen conjugates (5a, 5b, 5d, and 5e) and indomethacin (positive control) was used for the treatment of LPS-stimulated RAW264.7 macrophages. The Griess method was used to evaluate the nitrite content in cell supernatants. Significant differences indicated in the graphs are all in comparison to LPS-stimulated cells only. FIG. 2B is a bar graph showing cell viability test results. Cytotoxicity of the macrophages was evaluated using MTT assay. Values are mean±SD (n=3).

FIGS. 3A-3C are bar graphs showing the expression levels of IL-6 (FIG. 3A), TNF-α (FIG. 3B), and iNOS (FIG. 3C) mRNA in LPS-stimulated RAW264.7 macrophages. 40 μg/mL of indomethacin (positive control), 5a, 5b, 5d and 5e was used for treatment of LPS-stimulated RAW264.7 macrophages (10 ng/mL). RT-qPCR was used to measure mRNA levels of IL-6, TNF-α, and iNOS using the comparative method (2^{-ΔΔCT}). Significant differences indicated in the graphs are all in comparison to LPS-stimulated cells only (designated as ##). Values are means S.D. (n=3).

FIGS. 4A and 4B are 3D schematics showing the overlay of the bioactive conformation of Flurbiprofen with the Glide XP docked pose of the ligand in COX-1 crystal structure. PDB entry 3N8W (FIG. 4A) and the overlay of the bioactive conformation of SC-558 with the Glide XP docked pose of the ligand in COX-2 crystal structure, PDB entry 6COX (FIG. 4B).

FIGS. 5A and 5B are 2D (FIG. 5A) and 3D (FIG. 5B) schematics showing the binding interactions and docked pose of conjugate 5e in the COX-1 crystal structure. PDB: 3N8W. Interactions with Arg120, Leu531, and Val349 are shown.

FIGS. 6A and 6B are 2D (FIG. 6A) and 3D (FIG. 6B) schematics showing the binding interactions and docked pose of conjugate 5a in the COX-1 crystal structure, PDB: 3N8W. There is a loss of interactions with Leu531 and Val349, whereas interaction with Ser530 is observed. The choro group is oriented on the other side of the binding pocket and interacts with Ile523.

FIGS. 7A and 7B are 2D (FIG. 7A) and 3D (FIG. 7B) schematics showing the binding interactions and docked pose of conjugate 5a in the COX-2 crystal structure, PDB: 6COX. The choro group interacts with Pro86 and Val89.

FIG. 8 is a 3D schematic showing the overlay of docked (XP) poses of indomethacin and ibuprofen in the COX-2 crystal structure, PDB: 6COX. The carboxylic acid moiety of indomethacin was used as a seed group for conjugation. Enhanced COX-2 potency was achieved by targeting Pro86 and Val89.

FIG. 9 is a graph showing the QSAR plot of the observed versus predicted log[% inhibition of edema thickness for the conjugates at 10 mg/kg (rat body weight) indomethacin mol equivalent at 3 h effect].

FIG. 10 is a graph showing the QSAR plot of the observed versus predicted 1/property “% inhibition of peripheral analgesic properties for the conjugates at 10 mg/kg (rat body weight) indomethacin mol equivalent”.

FIG. 11 is a graph showing the QSAR plot of the observed versus predicted property “% protection for the central analgesic property of the conjugates at 10 mg/kg (rat body weight) indomethacin mol equivalent”.

### DETAILED DESCRIPTION OF THE INVENTION

### I. Definitions

It is to be understood that the disclosed compounds, compositions, and methods are not limited to specific synthetic methods, specific analytical techniques, or to particular reagents unless otherwise specified, and, as such, may vary. It is also to be understood that the terminology used herein is for the purpose of describing particular forms and embodiments only and is not intended to be limiting.

“Substituted,” as used herein, refers to all permissible substituents of the compounds or functional groups described herein. In the broadest sense, the permissible substituents include acyclic and cyclic, branched and unbranched, carbocyclic and heterocyclic, aromatic and nonaromatic substituents of organic compounds. Illustrative substituents include, but are not limited to, halogens, hydroxyl groups, or any other organic groupings containing any number of carbon atoms, preferably 1-14 carbon atoms, and optionally include one or more heteroatoms such as oxygen, sulfur, or nitrogen grouping in linear, branched, or cyclic structural formats. Representative substituents include a substituted or unsubstituted alkyl, a substituted or unsubstituted alkenyl, a substituted or unsubstituted alkynyl, a substituted or unsubstituted heterocyclyl, a substituted or unsubstituted phenyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a halogen, a hydroxyl, an alkoxy, a phenoxy, an aroxy, a silyl, a thiol, an alkylthio, a substituted alkylthio, a phenylthio, an arylthio, a cyano, an isocyano, a nitro, a substituted or unsubstituted carbonyl, a carboxyl, an amino, an amido, an oxo, a sulfinyl, a sulfonyl, a sulfonic acid, a phosphonium, a phosphanyl, a phosphoryl, a phosphonyl, an amino acid. Such a substituted or unsubstituted alkyl, a substituted or unsubstituted alkenyl, a substituted or unsubstituted alkynyl, a substituted or unsubstituted heterocyclyl, a substituted or unsubstituted phenyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a halogen, a hydroxyl, an alkoxy, a phenoxy, an aroxy, a silyl, a thiol, an alkylthio, a substituted alkylthio, a phenylthio, an arylthio, a cyano, an isocyano, a nitro, a substituted or unsubstituted carbonyl, a carboxyl, an amino, an amido, an oxo, a sulfinyl, a sulfonyl, a sulfonic acid, a phosphonium, a phosphanyl, a phosphoryl, a phosphonyl, and an amino acid can be further substituted.

Heteroatoms such as nitrogen may have hydrogen substituents and/or any permissible substituents of organic compounds described herein which satisfy the valences of the heteroatoms. It is understood that “substitution” or “substituted” includes the implicit proviso that such substitution is in accordance with permitted valence of the substituted atom and the substituent, and that the substitution results in a stable compound, i.e., a compound that does not spontaneously undergo transformation such as by rearrangement, cyclization, elimination, etc.

“Alkyl,” as used herein, refers to the radical of saturated aliphatic groups, including straight-chain alkyl groups, branched-chain alkyl, and cycloalkyl (alicyclic). In some forms, a straight chain or branched chain alkyl has 30 or fewer carbon atoms in its backbone (e.g., C₁-C₃₀ for straight chains, C₃-C₃₀ for branched chains), 20 or fewer, 15 or fewer, or 10 or fewer. Alkyl includes methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. Likewise, a cycloalkyl is a non-aromatic carbon-based ring composed of at least three carbon atoms, such as a nonaromatic monocyclic or nonaromatic polycyclic ring containing 3-30 carbon atoms, 3-20 carbon atoms, or 3-10 carbon atoms in their ring structure, and have 5, 6 or 7 carbons in the ring structure. Cycloalkyls containing a polycyclic ring system can have two or more non-aromatic rings in which two or more carbons are common to two adjoining rings (i.e., “fused cycloalkyl rings”). Examples of cycloalkyl groups include, but are not limited to, cyclopropyl, cyclobutyl, cyclopentyl, cyclohexyl, cycloheptyl, cyclooctanyl, etc.

“Substituted alkyl” refers to alkyl moieties having one or more substituents replacing a hydrogen on one or more carbons of the hydrocarbon backbone. Such substituents can be any substituents described above, e.g., halogen (such as fluorine, chlorine, bromine, or iodine), hydroxyl, carbonyl (such as a carboxyl, alkoxycarbonyl, formyl, or an acyl), thiocarbonyl (such as a thioester, a thioacetate, or a thioformate), aryl, alkoxyl, aralkyl, phosphonium, phosphanyl, phosphonyl, phosphoryl, phosphate, phosphonate, a phosphinate, amino, amido, amidine, imine, cyano, nitro, azido, oxo, sulfhydryl, thiol, alkylthio, silyl, sulfinyl, sulfate, sulfonate, sulfamoyl, sulfonamido, sulfonyl, heterocyclyl, an aromatic or heteroaromatic moiety. —NRR′, wherein R and R′ are independently hydrogen, alkyl, or aryl, and wherein the nitrogen atom is optionally quaternized; —SR, wherein R is a phosphonyl, a sulfinyl, a silyl a hydrogen, an alkyl, or an aryl; —CN; —NO₂; —COOH; carboxylate; —COR, —COOR, or —CON(R)₂, wherein R is hydrogen, alkyl, or aryl; imino, silyl, ether, haloalkyl (such as —CF₃, —CH₂—CF₃, —CCl₃); —CN; —NCOCOCH₂CH₂; —NCOCOCHCH; and —NCS; and combinations thereof.

It will be understood by those skilled in the art that the moieties substituted on the hydrocarbon chain can themselves be substituted, if appropriate. For instance, the substituents of a substituted alkyl may include halogen, hydroxy, nitro, thiols, amino, aralkyl, azido, imino, amido, phosphonium, phosphanyl, phosphoryl (including phosphonate and phosphinate), oxo, sulfonyl (including sulfate, sulfonamido, sulfamoyl and sulfonate), and silyl groups, as well as ethers, alkylthios, carbonyls (including ketones, aldehydes, carboxylates, and esters), haloalkyls, —CN and the like. Cycloalkyls can be substituted in the same manner.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths.

“Heteroalkyl,” as used herein, refers to straight or branched chain, or cyclic carbon-containing alkyl radicals, or combinations thereof, containing at least one heteroatom on the carbon backbone. Suitable heteroatoms include, but are not limited to, O, N, Si, P and S, wherein the nitrogen, phosphorous and sulfur atoms are optionally oxidized, and the nitrogen heteroatom is optionally quaternized. For example, the term “heterocycloalkyl group” is a cycloalkyl group as defined above where at least one of the carbon atoms of the ring is substituted with a heteroatom such as, but not limited to, nitrogen, oxygen, sulphur, or phosphorus.

The term “alkenyl” as used herein is a hydrocarbon group of from 2 to 24 carbon atoms and structural formula containing at least one carbon-carbon double bond. Alkenyl groups include straight-chain alkenyl groups, branched-chain alkenyl, and cycloalkenyl. A cycloalkenyl is a non-aromatic carbon-based ring composed of at least three carbon atoms and at least one carbon-carbon double bond, such as a nonaromatic monocyclic or nonaromatic polycyclic ring containing 3-30 carbon atoms and at least one carbon-carbon double bond, 3-20 carbon atoms and at least one carbon-carbon double bond, or 3-10 carbon atoms and at least one carbon-carbon double bond in their ring structure, and have 5, 6 or 7 carbons and at least one carbon-carbon double bond in the ring structure. Cycloalkenyls containing a polycyclic ring system can have two or more non-aromatic rings in which two or more carbons are common to two adjoining rings (i.e., “fused cycloalkenyl rings”) and contain at least one carbon-carbon double bond. Asymmetric structures such as (AB)C═C(C′D) are intended to include both the E and Z isomers. This may be presumed in structural formulae herein wherein an asymmetric alkene is present, or it may be explicitly indicated by the bond symbol C. The term “alkenyl” as used throughout the specification, examples, and claims is intended to include both “unsubstituted alkenyls” and “substituted alkenyls,” the latter of which refers to alkenyl moieties having one or more substituents replacing a hydrogen on one or more carbons of the hydrocarbon backbone. The term “alkenyl” also includes “heteroalkenyl.”

The term “substituted alkenyl” refers to alkenyl moieties having one or more substituents replacing one or more hydrogen atoms on one or more carbons of the hydrocarbon backbone. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, oxo, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

“Heteroalkenyl,” as used herein, refers to straight or branched chain, or cyclic carbon-containing alkenyl radicals, or combinations thereof, containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. For example, the term “heterocycloalkenyl group” is a cycloalkenyl group where at least one of the carbon atoms of the ring is substituted with a heteroatom such as, but not limited to, nitrogen, oxygen, sulphur, or phosphorus.

The term “alkynyl group” as used herein is a hydrocarbon group of 2 to 24 carbon atoms and a structural formula containing at least one carbon-carbon triple bond. Alkynyl groups include straight-chain alkynyl groups, branched-chain alkynyl, and cycloalkynyl. A cycloalkynyl is a non-aromatic carbon-based ring composed of at least three carbon atoms and at least one carbon-carbon triple bond, such as a nonaromatic monocyclic or nonaromatic polycyclic ring containing 3-30 carbon atoms and at least one carbon-carbon triple bond, 3-20 carbon atoms and at least one carbon-carbon triple bond, or 3-10 carbon atoms and at least one carbon-carbon triple bond in their ring structure, and have 5, 6 or 7 carbons and at least one carbon-carbon triple bond in the ring structure. Cycloalkynyls containing a polycyclic ring system can have two or more non-aromatic rings in which two or more carbons are common to two adjoining rings (i.e., “fused cycloalkynyl rings”) and contain at least one carbon-carbon triple bond. Asymmetric structures such as (AB)C≡C(C″D) are intended to include both the E and Z isomers. This may be presumed in structural formulae herein wherein an asymmetric alkyne is present, or it may be explicitly indicated by the bond symbol C. The term “alkynyl” as used throughout the specification, examples, and claims is intended to include both “unsubstituted alkynyls” and “substituted alkynyls,” the latter of which refers to alkynyl moieties having one or more substituents replacing a hydrogen on one or more carbons of the hydrocarbon backbone. The term “alkynyl” also includes “heteroalkynyl.”

The term “substituted alkynyl” refers to alkynyl moieties having one or more substituents replacing one or more hydrogen atoms on one or more carbons of the hydrocarbon backbone. Such substituents can be any substituents described above. e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

“Heteroalkynyl,” as used herein, refers to straight or branched chain, or cyclic carbon-containing alkynyl radicals, or combinations thereof, containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. For example, the term “heterocycloalkynyl group” is a cycloalkynyl group where at least one of the carbon atoms of the ring is substituted with a heteroatom such as, but not limited to, nitrogen, oxygen, sulphur, or phosphorus.

“Aryl,” as used herein, refers to C₅-C₂₆-membered aromatic or fused aromatic ring systems. Examples of aromatic groups are benzene, naphthalene, anthracene, phenanthrene, chrysene, pyrene, corannulene, coronene, etc.

The term “substituted aryl” refers to an aryl group, wherein one or more hydrogen atoms on one or more aromatic rings are substituted with one or more substituents including, but not limited to, halogen, azide, alkyl, aralkyl, alkenyl, alkynyl, cycloalkyl, hydroxyl, alkoxy, carbonyl (such as a ketone, aldehyde, carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, imino, alkylthio, sulfate, sulfonate, sulfamoyl, sulfoxide, sulfonamido, sulfonyl, heterocyclyl, alkylaryl, haloalkyl (such as CF₃, —CH₂—CF₃, —CCl₃), —CN, aryl, heteroaryl, and combinations thereof.

“Heterocycle” and “heterocyclyl” are used interchangeably, and refer to a cyclic radical attached via a ring carbon or nitrogen atom of a non-aromatic monocyclic or polycyclic ring containing 3-30 ring atoms, 3-20 ring atoms, 3-10 ring atoms, or 5-6 ring atoms, where each ring contains carbon and one to four heteroatoms each selected from the group consisting of non-peroxide oxygen, sulfur, and N(Y) wherein Y is absent or is H, O, C₁-C₁₀ alkyl, phenyl or benzyl, and optionally containing 1-3 double bonds and optionally substituted with one or more substituents. Heterocyclyl are distinguished from heteroaryl by definition. Heterocycles can be a heterocycloalkyl, a heterocycloalkenyl, a heterocycloalkynyl, etc, such as piperazinyl, piperidinyl, piperidonyl, 4-piperidonyl, dihydrofuro[2,3-b]tetrahydrofuran, morpholinyl, piperazinyl, piperidinyl, piperidonyl, 4-piperidonyl, piperonyl, pyranyl, 2H-pyrrolyl, 4H-quinolizinyl, quinuclidinyl, tetrahydrofuranyl, 6H-1,2,5-thiadiazinyl. Heterocyclic groups can optionally be substituted with one or more substituents as defined above for alkyl and aryl.

The term “heteroaryl” refers to C₅-C₂₆-membered aromatic or fused aromatic ring systems, in which one or more carbon atoms on one or more aromatic ring structures have been substituted with a heteroatom. Suitable heteroatoms include, but are not limited to, oxygen, sulfur, and nitrogen. Examples of heteroaryl groups pyrrole, furan, thiophene, imidazole, oxazole, thiazole, triazole, tetrazole, pyrazole, pyridine, pyrazine, pyridazine and pyrimidine, and the like. Examples of heteroaryl rings include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, naphthyridinyl, octahydroisoquinolinyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, pteridinyl, purinyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolyl, quinazolinyl, quinolinyl, quinoxalinyl, tetrahydroisoquinolinyl, tetrahydroquinolinyl, tetrazolyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. One or more of the rings can be substituted as defined below for “substituted heteroaryl.”

The term “substituted heteroaryl” refers to a heteroaryl group in which one or more hydrogen atoms on one or more heteroaromatic rings are substituted with one or more substituents including, but not limited to, halogen, azide, alkyl, aralkyl, alkenyl, alkynyl, cycloalkyl, hydroxyl, alkoxy, carbonyl (such as a ketone, aldehyde, carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, imino, alkylthio, sulfate, sulfonate, sulfamoyl, sulfoxide, sulfonamido, sulfonyl, heterocyclyl, alkylaryl, haloalkyl (such as CF₃, —CH₂—CF₃, —CCl₃), —CN, aryl, heteroaryl, and combinations thereof.

The term “polyaryl” refers to a chemical moiety that includes two or more fused aryl groups. When two or more fused heteroaryl groups are involved, the chemical moiety can be referred to as a “polyheteroaryl.”

The term “substituted polyaryl” refers to a polyaryl in which one or more of the aryls are substituted, with one or more substituents including, but not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfoxide, sulfonamido, sulfonyl, heterocyclyl, alkylaryl, haloalkyl, —CN, aryl, heteroaryl, and combinations thereof. When a polyheteroaryl is involved, the chemical moiety can be referred to as a “substituted polyheteroaryl.”

The term “cyclic ring” or “cyclic group” refers to a substituted or unsubstituted monocyclic ring or a substituted or unsubstituted polycyclic ring (such as those formed from single or fused ring systems), such as a substituted or unsubstituted cycloalkyl, a substituted or unsubstituted cycloalkenyl, a substituted or unsubstituted cycloalkynyl, or a substituted or unsubstituted heterocyclyl, that have from three to 30 carbon atoms, as geometric constraints permit. The substituted cycloalkyls, cycloalkenyls, cycloalkynyls, and heterocyclyls are substituted as defined above for the alkyls, alkenyls, alkynyls, and heterocyclyls, respectively.

The term “aralkyl” as used herein is an aryl group or a heteroaryl group having an alkyl, alkynyl, or alkenyl group as defined above attached to the aromatic group, such as an aryl, a heteroaryl, a polyaryl, or a polyheteroaryl. An example of an aralkyl group is a benzyl group.

The terms “alkoxyl” or “alkoxy,” “aroxy” or “aryloxy,” generally describe compounds represented by the formula —OR^{v}, wherein R^{v} includes, but is not limited to, a substituted or unsubstituted alkyl, a substituted or unsubstituted alkenyl, a substituted or unsubstituted alkynyl, a substituted or unsubstituted cycloalkyl, a substituted or unsubstituted heterocyclyl, a substituted or unsubstituted cycloalkenyl, a substituted or unsubstituted heterocycloalkenyl, a substituted or unsubstituted aryl, a substituted or unsubstituted heteroaryl, a substituted or unsubstituted polyaryl, a substituted or unsubstituted polyheteroaryl, a substituted or unsubstituted arylalkyl, a substituted or unsubstituted heteroalkyl, a substituted or unsubstituted alkylaryl, a substituted or unsubstituted alkylheteroaryl, a substituted or unsubstituted aralkyl, a substituted or unsubstituted carbonyl, a phosphonium, a phosphanyl, a phosphonyl, a sulfinyl, a silyl, a thiol, an amido, and an amino. Exemplary alkoxyl groups include methoxy, ethoxy, propyloxy, tert-butoxy and the like. A “lower alkoxy” group is an alkoxy group containing from one to six carbon atoms. An “ether” is two functional groups covalently linked by an oxygen as defined below. Accordingly, the substituent of an alkyl that renders that alkyl an ether is or resembles an alkoxyl, such as can be represented by one of —O-alkyl, —O-alkenyl, —O-alkynyl, —O-arakyl, —O-aryl, —O-heteroaryl, —O-polyaryl, —O-polyheteroaryl, —O-heterocyclyl, etc.

The term “substituted alkoxy” refers to an alkoxy group having one or more substituents replacing one or more hydrogen atoms on one or more carbons of the alkoxy backbone. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, alkylthio, oxo, sulfate, sulfonate, sulfamoyl, sulfonamido, sulfonyl, heterocyclyl, alkylaryl, haloalkyl, —CN, aryl, heteroaryl, and combinations thereof.

The term “ether” as used herein is represented by the formula A²OA¹, where A² and A¹ can be, independently, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted aralkyl, a substituted or unsubstituted polyaryl, a substituted or unsubstituted polyheteroaryl, a phosphonium, a phosphanyl, a phosphonyl, a sulfinyl, a silyl, a thiol, a substituted or unsubstituted carbonyl, an alkoxy, an amido, or an amino, described above.

The term “polyether” as used herein is represented by the formula:

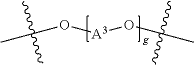

where A³ can be, independently, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted aralkyl, a substituted or unsubstituted polyaryl, a substituted or unsubstituted polyheteroaryl, a phosphonium, a phosphanyl, a substituted or unsubstituted carbonyl, an alkoxy, an amido, or an amino, described above; g can be a positive integer from 1 to 30.

The term “phenoxy” is art recognized and refers to a compound of the formula —OR^{v} wherein R^{v} is C₆H₅ (i.e., —O—C₆H₅). One of skill in the art recognizes that a phenoxy is a species of the aroxy genus.

The term “substituted phenoxy” refers to a phenoxy group, as defined above, having one or more substituents replacing one or more hydrogen atoms on one or more carbons of the phenyl ring. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, and combinations thereof.

The terms “aroxy” and “aryloxy.” as used interchangeably herein, are represented by —O-aryl or —O-heteroaryl, wherein aryl and heteroaryl are as defined herein.

The terms “substituted aroxy” and “substituted aryloxy,” as used interchangeably herein, represent —O-aryl or —O-heteroaryl, having one or more substituents replacing one or more hydrogen atoms on one or more ring atoms of the aryl and heteroaryl, as defined herein. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

The term “amino” as used herein includes the group

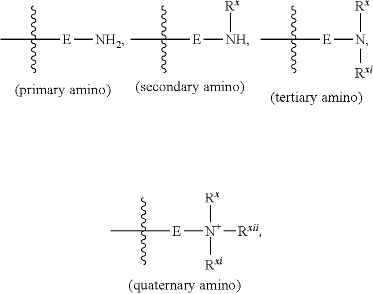

wherein, E is absent, or E is substituted or unsubstituted alkyl, substituted or unsubstituted alkenyl, substituted or unsubstituted alkynyl, substituted or unsubstituted aralkyl, substituted or unsubstituted aryl, substituted or unsubstituted heteroaryl, a substituted or unsubstituted polyaryl, a substituted or unsubstituted polyheteroaryl, substituted or unsubstituted heterocyclyl, wherein independently of E, R^{x}, R^{xi}, and R^{xii} each independently represent a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₙ—R′″; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. The term “quaternary amino” also includes the groups where the nitrogen, R^{x}, R^{xi}, and R^{xii} with the N⁺ to which they are attached complete a heterocyclyl or heteroaryl having from 3 to 14 atoms in the ring structure. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The terms “amide” or “amido” are used interchangeably, refer to both “unsubstituted amido” and “substituted amido” and are represented by the general formula:

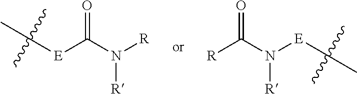

wherein, E is absent, or E is a substituted or unsubstituted alkyl, a substituted or unsubstituted alkenyl, a substituted or unsubstituted alkynyl, a substituted or unsubstituted aralkyl, a substituted or unsubstituted aryl, substituted or unsubstituted heteroaryl, a substituted or unsubstituted polyaryl, a substituted or unsubstituted polyheteroaryl, or a substituted or unsubstituted heterocyclyl, wherein independently of E, R and R′ each independently represent a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘ—R′″, or R and R′ taken together with the N atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. In some forms, when E is oxygen, a carbamate is formed. It is understood by those of ordinary skill in the art, that the E groups listed above are divalent (e.g., methylene, ethane-1,2-diyl, ethene-1,2-diyl, 1,4-phenylene, cyclohexane-1,2-diyl).

“Carbonyl,” as used herein, is art-recognized and includes such moieties as can be represented by the general formula:

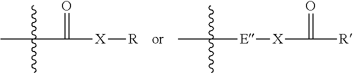

wherein X is a bond, or represents an oxygen or a sulfur, and R represents a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, an amido, an amino, or —(CH₂)ₘ—R″, or a pharmaceutical acceptable salt; E″ is absent, or E″ is a substituted or unsubstituted alkyl, a substituted or unsubstituted alkenyl, a substituted or unsubstituted alkynyl, a substituted or unsubstituted aralkyl, a substituted or unsubstituted aryl, a substituted or unsubstituted heteroaryl, a substituted or unsubstituted polyaryl, a substituted or unsubstituted polyheteroaryl, a substituted or unsubstituted heterocyclyl; R′ represents a hydrogen, a substituted or unsubstituted alkyl, a substituted or unsubstituted alkenyl, a substituted or unsubstituted alkynyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, an amido, an amino, or —(CH₂)ₘ—R″; R″ represents a hydroxyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, and combinations thereof. It is understood by those of ordinary skill in the art, that the E″ groups listed above are divalent (e.g., methylene, ethane-1,2-diyl, ethene-1,2-diyl, 1,4-phenylene, cyclohexane-1,2-diyl). Where X is oxygen and R is defined as above, the moiety is also referred to as a carboxyl group. When X is oxygen and R is hydrogen, the formula represents a “carboxylic acid.” Where X is oxygen and R′ is hydrogen, the formula represents a “formate.” Where X is oxygen and R or R′ is not hydrogen, the formula represents an “ester.” In general, where the oxygen atom of the above formula is replaced by a sulfur atom, the formula represents a “thiocarbonyl” group. Where X is sulfur and R or R′ is not hydrogen, the formula represents a “thioester.” Where X is sulfur and R is hydrogen, the formula represents a “thiocarboxylic acid.” Where X is sulfur and R′ is hydrogen, the formula represents a “thioformate.” Where X is a bond and R is not hydrogen, the above formula represents a “ketone.” Where X is a bond and R is hydrogen, the above formula represents an “aldehyde.”

The term “phosphanyl” is represented by the formula

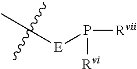

wherein, E is absent, or E is a substituted or unsubstituted alkyl, a substituted or unsubstituted alkenyl, a substituted or unsubstituted alkynyl, a substituted or unsubstituted aralkyl, a substituted or unsubstituted cycloalkyl, a substituted or unsubstituted aryl, a substituted or unsubstituted heteroaryl, a substituted or unsubstituted polyaryl, a substituted or unsubstituted polyheteroaryl, a substituted or unsubstituted heterocyclyl, wherein independently of E, R^{vi} and R^{vii} each independently represent a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g., a substituted or unsubstituted alkylaryl, a substituted or unsubstituted arylalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘ—R′″, or R^{vi} and R^{vii} taken together with the P atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “phosphonium” is represented by the formula

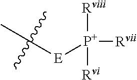

wherein, E is absent, or E is a substituted or unsubstituted alkyl, a substituted or unsubstituted alkenyl, a substituted or unsubstituted alkynyl, a substituted or unsubstituted aralkyl, a substituted or unsubstituted cycloalkyl, a substituted or unsubstituted aryl, a substituted or unsubstituted heteroaryl, a substituted or unsubstituted polyaryl, a substituted or unsubstituted polyheteroaryl, a substituted or unsubstituted heterocyclyl, wherein independently of E, R^{vi}, R^{vii}, and R^{viii} each independently represent a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘ—R′″, or R^{vi}, R^{vii}, and R^{viii} taken together with the P⁺ atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “phosphonyl” is represented by the formula

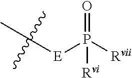

wherein E is absent, or E is a substituted or unsubstituted alkyl, a substituted or unsubstituted alkenyl, a substituted or unsubstituted alkynyl, a substituted or unsubstituted aralkyl (e.g., a substituted or unsubstituted alkylaryl, a substituted or unsubstituted arylalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, oxygen, alkoxy, aroxy, or substituted alkoxy or substituted aroxy, wherein, independently of E, R^{vi} and R^{vii} are independently a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘ—R′″, or R^{vi} and R^{vii} taken together with the P atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “phosphoryl” defines a phosphonyl in which E is absent, oxygen, alkoxy, aroxy, substituted alkoxy or substituted aroxy, as defined above, and independently of E, R^{vi} and R^{vii} are independently hydroxyl, alkoxy, aroxy, substituted alkoxy or substituted aroxy, as defined above. When E is oxygen, the phosphoryl cannot be attached to another chemical species, such as to form an oxygen-oxygen bond, or other unstable bonds, as understood by one of ordinary skill in the art. When E, R^{vi} and R^{vii} are substituted, the substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “sulfinyl” is represented by the formula

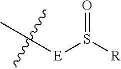

wherein E is absent, or E is a substituted or unsubstituted alkyl, a substituted or unsubstituted alkenyl, a substituted or unsubstituted alkynyl, a substituted or unsubstituted aralkyl (e.g., a substituted or unsubstituted alkylaryl, a substituted or unsubstituted arylalkyl, etc.), a substituted or unsubstituted aryl, a substituted or unsubstituted heteroaryl, a substituted or unsubstituted heterocyclyl, a substituted or unsubstituted polyaryl, a substituted or unsubstituted polyheteroaryl, wherein independently of E, R represents a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a silyl, a thiol, an amido, an amino, or —(CH₂)ₘ—R′″, or E and R taken together with the S atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “sulfonyl” is represented by the formula

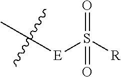

wherein E is absent, or E is a substituted or unsubstituted alkyl, a substituted or unsubstituted alkenyl, a substituted or unsubstituted alkynyl, a substituted or unsubstituted aralkyl (e.g., a substituted or unsubstituted alkylaryl, a substituted or unsubstituted arylalkyl, etc.), a substituted or unsubstituted aryl, a substituted or unsubstituted heteroaryl, a substituted or unsubstituted heterocyclyl, a substituted or unsubstituted polyaryl, a substituted or unsubstituted polyheteroaryl, wherein independently of E. R represents a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, an amido, an amino, or —(CH₂)ₘ—R′″, or E and R taken together with the S atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “sulfonic acid” refers to a sulfonyl, as defined above, wherein R is hydroxyl, and E is absent, or E is substituted or unsubstituted cycloalkyl, substituted or unsubstituted heterocyclyl, substituted or unsubstituted alkylaryl, substituted or unsubstituted arylalkyl, substituted or unsubstituted aryl, a substituted or unsubstituted polyaryl, a substituted or unsubstituted polyheteroaryl, or substituted or unsubstituted heteroaryl. Such substituents can be any substituents described above. e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “sulfate” refers to a sulfonyl, as defined above, wherein E is absent, oxygen, alkoxy, aroxy, substituted alkoxy or substituted aroxy, as defined above, and R is independently hydroxyl, alkoxy, aroxy, substituted alkoxy or substituted aroxy, as defined above. When E is oxygen, the sulfate cannot be attached to another chemical species, such as to form an oxygen-oxygen bond, or other unstable bonds, as understood by one of ordinary skill in the art. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “sulfonate” refers to a sulfonyl, as defined above, wherein E is oxygen, alkoxy, aroxy, substituted alkoxy or substituted aroxy, as defined above, and R is independently hydrogen, substituted or unsubstituted alkyl, substituted or unsubstituted alkenyl, substituted or unsubstituted alkynyl, substituted or unsubstituted amino, substituted or unsubstituted cycloalkyl, substituted or unsubstituted heterocyclyl, substituted or unsubstituted aralkyl, substituted or unsubstituted alkylaryl, substituted or unsubstituted arylalkyl, substituted or unsubstituted aryl, substituted or unsubstituted heteroaryl, a substituted or unsubstituted polyaryl, a substituted or unsubstituted polyheteroaryl, —(CH₂)ₘ—R′″, R′″ represents a hydroxy group, substituted or unsubstituted carbonyl group, an aryl, a cycloalkyl ring, a cycloalkenyl ring, a heterocycle, an amido, an amino, or a polycycle; and m is zero or an integer ranging from 1 to 8. When E is oxygen, sulfonate cannot be attached to another chemical species, such as to form an oxygen-oxygen bond, or other unstable bonds, as understood by one of ordinary skill in the art. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “sulfamoyl” refers to a sulfonamide or sulfonamide represented by the formula

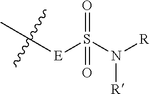

wherein E is absent, or E is substituted or unsubstituted alkyl, a substituted or unsubstituted alkenyl, a substituted or unsubstituted alkynyl, a substituted or unsubstituted aralkyl (e.g., a substituted or unsubstituted alkylaryl, a substituted or unsubstituted cycloalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, wherein independently of E. R and R′ each independently represent a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, an amido, an amino, or —(CH₂)ₘ—R′″, or R and R′ taken together with the N atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “silyl group” as used herein is represented by the formula —SiRR′R,″ where R, R′, and R″ can be, independently, a hydrogen, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted aralkyl (e.g. a substituted or unsubstituted alkylaryl, a substituted or unsubstituted arylalkyl, etc.), a substituted or unsubstituted polyaryl, a substituted or unsubstituted polyheteroaryl, a substituted or unsubstituted carbonyl, a phosphonium, a phosphanyl, a phosphonyl, a sulfinyl, a thiol, an amido, an amino, an alkoxy, or an oxo, described above. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

The terms “thiol” are used interchangeably and are represented by —SR, where R can be a hydrogen, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted aralkyl (e.g. a substituted or unsubstituted alkylaryl, a substituted or unsubstituted arylalkyl, etc.), a substituted or unsubstituted polyaryl, a substituted or unsubstituted polyheteroaryl, a substituted or unsubstituted carbonyl, a phosphonium, a phosphanyl, an amido, an amino, an alkoxy, an oxo, a phosphonyl, a sulfinyl, or a silyl, described above. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

The disclosed compounds and substituent groups, can, independently, possess two or more of the groups listed above. For example, if the compound or substituent group is a straight chain alkyl group, one of the hydrogen atoms of the alkyl group can be substituted with a hydroxyl group, an alkoxy group, etc. Depending upon the groups that are selected, a first group can be incorporated within second group or, alternatively, the first group can be pendant (i.e., attached) to the second group. For example, with the phrase “an alkyl group comprising an ester group,” the ester group can be incorporated within the backbone of the alkyl group. Alternatively, the ester can be attached to the backbone of the alkyl group. The nature of the group(s) that is (are) selected will determine if the first group is embedded or attached to the second group.

The compounds and substituents can be substituted, independently, with the substituents described above in the definition of “substituted.”

The numerical ranges disclose individually each possible number that such a range could reasonably encompass, as well as any sub-ranges and combinations of sub-ranges encompassed therein. For example, in a given range carbon range of C₃-C₉, the range also discloses C₃, C₄, C₅, C₆, C₇, C₈, and C₉, as well as any subrange between these numbers (for example, C₄-C₆), and any possible combination of ranges possible between these values. In yet another example, a given temperature range may be from about 25° C. to 30° C., where the range also discloses temperatures that can be selected independently from about 25, 26, 27, 28, 29, and 30° C., as well as any range between these numbers (for example, 26 to 28° C.), and any possible combination of ranges between these values.

Use of the term “about” is intended to describe values either above or below the stated value, which the term “about” modifies, to be within a range of approximately +/−10%. When the term “about” is used before a range of numbers (i.e., about 1-5) or before a series of numbers (i.e., about 1, 2, 3, 4, etc.) it is intended to modify both ends of the range of numbers and/or each of the numbers recited in the entire series, unless specified otherwise.

The disclosed compounds and substituent groups, can, independently, possess two or more of the groups listed above. For example, if the compound or substituent group is a straight chain alkyl group, one of the hydrogen atoms of the alkyl group can be substituted with a hydroxyl group, an alkoxy group, etc. Depending upon the groups that are selected, a first group can be incorporated within second group or, alternatively, the first group can be pendant (i.e., attached) to the second group. For example, with the phrase “an alkyl group comprising an ester group.” the ester group can be incorporated within the backbone of the alkyl group. Alternatively, the ester can be attached to the backbone of the alkyl group. The nature of the group(s) that is (are) selected will determine if the first group is embedded or attached to the second group.

The compounds and substituents can be substituted with, independently, with the substituents described above in the definition of “substituted.”

### II. Compositions

Described herein are synthetic compounds having anti-inflammatory properties and should be suitable for use in the prevention or treatment of a variety of anti-inflammatory disease or disorders, and to provide analgesia in subjects in need thereof. In some forms, these compounds also have peripheral and/or central analgesic properties.

The compounds disclosed herein are derivatives of FDA-approved anti-inflammatory drugs (also referred to herein as “parent drugs”), such as ibuprofen and indomethacin. Such compounds can be formed by conjugation of a substituted or unsubstituted aryl group to the parent drug via azide-alkyne click chemistry (and thus the compounds are also referred to herein as “conjugates”). Accordingly, the formed conjugate contains a moiety of the parent drug that corresponds to the unreacted portion of the parent drug, a triazolyl heterocycle, and the substituted or unsubstituted aryl group. Optionally, the parent drug moiety and the triazolyl heterocycle are linked by a linker.

Generally, the conjugates disclosed herein show anti-inflammatory properties and and/or analgesic properties with similar or higher efficacy and/or potency compared with their respective parent drugs. For example, the conjugates show a similar or higher anti-inflammatory efficacy, as indicated by a similar or greater reduction of edema thickness of a body part of a subject in need thereof, compared with a control treated with their respective parent drugs. Without being bound to any theories, it is believed that the disclosed compounds can suppress the production of NO and/or inflammatory cytokines, such as IL-6, TNF-α, and iNOS, and thereby provide anti-inflammatory properties. Additionally, the conjugates may have a similar or higher analgesic efficiency, as indicated by a similar or more reduction of peripheral and/or central pain of a subject in need thereof, compared with a control treated with their respective parent drugs.

Further, the overall structure of the disclosed conjugates can overcome many disadvantages of the current anti-inflammatory drugs, in particular the current NSAIDs. For example, 2-(4-isobutylphenyl)propanoic acid, known as ibuprofen (Ibu), is extensively used as an over the counter NSAIDs in musculoskeletal disorders, osteoarthritis, and rheumatoid arthritis and as an antipyretic. Indomethacin (Indo) is also extensively used as a potent prescription NSAID. However, both Ibu and Indo are associated with several adverse effects in chronic use, such as gastrointestinal and ulcerogenic side effects, which restricts their use, in particular in individuals having other health complications. In contrast, the conjugated disclosed herein show anti-inflammatory properties and/or analgesic properties with no or reduced adverse effects, for example, without ulcerogenic effects. Additionally, in some forms, the disclosed conjugates can selectively inhibit COX-2 over COX-1. The therapeutic anti-inflammatory action of NSAIDs is produced by the inhibition of COX-2, while the undesired side effects arise from inhibition of COX-1 activity.

Pharmaceutical compositions and formulations containing the conjugates are also disclosed.

### A. Conjugates

The disclosed conjugates can have the structure of Formula I:

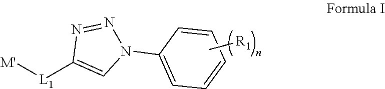

wherein: (i) R₁ is a halide, an unsubstituted alkyl, a substituted alkyl, an alkoxy, a nitro, a carbonyl, a hydroxyl, an amino, an amido, a cyano, a thio, an unsubstituted alkenyl, a substituted alkenyl, an unsubstituted alkynyl, a substituted alkynyl, an unsubstituted alkylaryl, a substituted alkylaryl, an unsubstituted aryl, or a substituted aryl;
(ii) n is an integer from 0 to 5, from 0 to 4, from 0 to 3, or 1 or 2, such as 1;
(iii) L₁ is

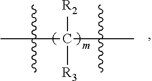

m is an integer from 0 to 6, R₂ and R₃ are independently H, an unsubstituted alkyl, or a substituted alkyl;
(iv) M′ is a parent drug moiety; and
(v) each substituent is independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a carbonyl, an alkoxy, a halogen, a hydroxyl, a phenoxy, a thiol, an alkylthio, a phenylthio, an arylthio, a cyano, an isocyano, a nitro, a carboxyl, an amino, an amido, an oxo, a silyl, a sulfinyl, a sulfonyl, a sulfonic acid, a phosphonium, a phosphanyl, a phosphoryl, or a phosphonyl.

The alkyl can be a linear alkyl, a branched alkyl, or a cyclic alkyl (either monocyclic or polycyclic). The terms “cyclic alkyl” and “cycloalkyl” are used interchangeably herein. Exemplary alkyl include a linear C₁-C₃₀ alkyl, a branched C₄-C₃₀ alkyl, a cyclic C₃-C₃₀ alkyl, a linear C₁-C₂₀ alkyl, a branched C₄-C₂₀ alkyl, a cyclic C₃-C₂₀ alkyl, a linear C₁-C₁₀ alkyl, a branched C₄-C₁₀ alkyl, a cyclic C₃-C₁₀ alkyl, a linear C₁-C₆ alkyl, a branched C₄-C₆ alkyl, a cyclic C₃-C₆ alkyl, a linear C₁-C₄ alkyl, cyclic C₃-C₄ alkyl, such as a linear C₁-C₁₀, C₁-C₉, C₁-C₈, C₁-C₇, C₁-C₆, C₁-C₅, C₁-C₄, C₁-C₃, or C₁-C₂ alkyl group, a branched C₃-C₉, C₃-C₉, C₃-C₈, C₃-C₇, C₃-C₆, C₃-C₅, or C₃-C₄ alkyl group, or a cyclic C₃-C₉, C₃-C₉, C₃-C₈, C₃-C₇, C₃-C₆, C₃-C₅, or C₃-C₄ alkyl group. The cyclic alkyl can be a monocyclic or polycyclic alkyl, such as a C₄-C₃₀, C₄-C₂₅, C₄-C₂₀, C₄-C₁₅, C₄-C₁₆, C₄-C₁₅, C₄-C₁₄, C₄-C₁₃, C₄-C₁₂, C₄-C₁₀, C₄-C₉, C₄-C₆, C₄-C₇, C₄-C₆, or C₄-C₅ monocyclic or polycyclic alkyl group.

The alkenyl can be a linear alkenyl, a branched alkenyl, or a cyclic alkenyl (either monocyclic or polycyclic). The terms “cyclic alkenyl” and “cycloalkenyl” are used interchangeably herein. Exemplary alkenyl include a linear C₂-C₃₀ alkenyl, a branched C₄-C₃₀ alkenyl, a cyclic C₃-C₃₀ alkenyl, a linear C₂-C₂₀ alkenyl, a branched C₄-C₂₀ alkenyl, a cyclic C₃-C₂₀ alkenyl, a linear C₂-C₁₀ alkenyl, a branched C₄-C₁₀ alkenyl, a cyclic C₃-C₁₀ alkenyl, a linear C₂-C₆ alkenyl, a branched C₄-C₆ alkenyl, a cyclic C₃-C₆ alkenyl, a linear C₂-C₄ alkenyl, cyclic C₃-C₄ alkenyl, such as a linear C₂-C₁₀, C₂-C₉, C₂-C₈, C₂-C₇, C₂-C₆, C₂-C₅, C₂-C₄, C₂-C₃, C₂ alkenyl group, a branched C₃-C₉, C₃-C₉, C₃-C₈, C₃-C₇, C₃-C₆, C₃-C₅, C₃-C₄ alkenyl group, or a cyclic C₃-C₉, C₃-C₉, C₃-C₈, C₃-C₇, C₃-C₆, C₃-C₅, C₃-C₄ alkenyl group. The cyclic alkenyl can be a monocyclic or polycyclic alkenyl, such as a C₄-C₃₀, C₄-C₂₅, C₄-C₂₀, C₄-C₁₈, C₄-C₁₆, C₄-C₁₅, C₄-C₁₄, C₄-C₁₃, C₄-C₁₂, C₄-C₁₀, C₄-C₉, C₄-C₈, C₄-C₇, C₄-C₆, or C₄- C₅ monocylcic or polycyclic alkenyl group.

The alkynyl can be a linear alkynyl, a branched alkynyl, or a cyclic alkynyl (either monocyclic or polycyclic). The terms “cyclic alkynyl” and “cycloalkynyl” are used interchangeably herein. Exemplary alkynyl include a linear C₂-C₃₀ alkynyl, a branched C₄-C₃₀ alkynyl, a cyclic C₃-C₃₀ alkynyl, a linear C₂-C₂₀ alkynyl, a branched C₄-C₂₀ alkynyl, a cyclic C₃-C₂₀ alkynyl, a linear C₂-C₁₀ alkynyl, a branched C₄-C₁₀ alkynyl, a cyclic C₃-C₁₀ alkynyl, a linear C₂-C₆ alkynyl, a branched C₄-C₆ alkynyl, a cyclic C₃-C₆ alkynyl, a linear C₂-C₄ alkynyl, cyclic C₃-C₄ alkynyl, such as a linear C₂-C₁₀, C₂-C₉, C₂-C₈, C₂-C₇, C₂-C₆, C₂-C₅, C₂-C₄, C₂-C₃, C₂ alkynyl group, a branched C₃-C₉, C₃-C₉, C₃-C₈, C₃-C₇, C₃-C₆, C₃-C₅, C₃-C₄ alkynyl group, or a cyclic C₃-C₉, C₃-C₉, C₃-C₈, C₃-C₇, C₃-C₆, C₃-C₅, C₃-C₄ alkynyl group. The cyclic alkynyl can be a monocyclic or polycyclic alkynyl, such as a C₄-C₃₀, C₄-C₂₅, C₄-C₂₀, C₄-C₁₈, C₄-C₁₆, C₄-C₁₅, C₄-C₁₄, C₄-C₁₃, C₄-C₁₂, C₄-C₁₀, C₄-C₉, C₄- C₈, C₄-C₇, C₄-C₆, or C₄-C₅ monocyclic or polycyclic alkynyl group.

It is understood that any of the exemplary alkyl, alkenyl, and alkynyl groups can be heteroalkyl, heteroalkenyl, and heteroalkynyl, respectively.

The aryl group can be a C₅-C₃₀ aryl, a C₅-C₂₀ aryl, a C₅-C₁₂ aryl, a C₅-C₁₁ aryl, a C₅-C₉ aryl, a C₆-C₂₀ aryl, a C₆-C₁₂ aryl, a C₆-C₁₁ aryl, or a C₆-C₉ aryl. It is understood that the aryl can be a heteroaryl, such as a C₅-C₃₀ heteroaryl, a C₅-C₂₀ heteroaryl, a C₅-C₁₂ heteroaryl, a C₅-C₁₁ heteroaryl, a C₅-C₉ heteroaryl, a C₆-C₃₀ heteroaryl, a C₆-C₂₀ heteroaryl, a C₆-C₂ heteroaryl, a C₆-C₁₁ heteroaryl, or a C₆-C₉ heteroaryl. For any of Formulae I, Ia, II, III, and IV, the polyaryl group can be a C₁₀-C₃₀ polyaryl, a C₁₀-C₂₀ polyaryl, a C₁₀-C₁₂ polyaryl, a C₁₀-C₁₁ polyaryl, or a C₁₂-C₂₀ polyaryl. It is understood that the aryl can be a polyheteroaryl, such as a C₁₀-C₃₀ polyheteroaryl, a C₁₀-C₂₀ polyheteroaryl, a C₁₀-C₁₂ polyheteroaryl, a C₁₀-C₁₁ polyheteroaryl, or a C₁₂-C₂₀ polyheteroaryl.

In some forms of Formula I, the substituents can be independently an unsubstituted alkyl, an unsubstituted aryl, an unsubstituted aralkyl, a carbonyl, an alkoxy, a halogen, a hydroxyl, a cyano, a nitro, an amino, an amido, or an oxo.

In some forms of Formula I, the substituents can be independently an unsubstituted alkyl (e.g. a linear C₁-C₆, C₁-C₅, C₁-C₄, C₁-C₃, C₁-C₂ alkyl group, a branched C₃-C₆, C₃-C₅, C₃-C₄ alkyl group, or a cyclic C₃-C₆, C₃-C₅, C₃-C₄ alkyl group), an unsubstituted aryl, an unsubstituted aralkyl, a carbonyl (e.g. a carboxyl), an alkoxy, a halogen, a hydroxyl, an amino, or an amido.

The parent drug moiety M′ of the disclosed conjugates can be the unreacted portion of any suitable parent drugs, such as the unreacted portion of a NSAID that has a carboxyl group, a primary or secondary amino group, and/or a hydroxyl group. “Unreacted portion” refers to the portion of the parent drug that remain unchanged after the conjugation reactions forming the conjugates. Generally, suitable parent drugs for forming the conjugates contain a reactive group that can be converted and attach to an alkynyl group, such as a carboxyl group, A primary or secondary amino group, and/or a hydroxyl group. The alkynyl group can then react with an azide-modified aryl (with or without additional substituents) to form the conjugates.

Preferably, the parent drug moiety M′ of the conjugates is the unreacted portion of a NSAID, such as a NSAID having a carboxyl group as the reactive group, a primary or secondary amino group, and/or a hydroxyl group. In some forms, the parent drug moiety M′ of the conjugates is the unreacted portion of a NSAID having a carboxyl group, for example, ibuprofen, indomethacin, aspirin, mefenamic acid, diclofenac, naproxen, sulindac, salsalate, etodolac, ketoprofen, ketorolac, flurbiprofen, oxaprozin, diflunisal, meclofenamate, fenoprofen, and tolmetin. In some forms, the parent drug moiety M′ of the conjugates is the unreacted portion of a NSAID having a primary or secondary amino group as the reactive group, for example, celecoxib and acetaminophen. In some forms, the parent drug moiety M′ of the conjugates is the unreacted portion of a NSAID having a hydroxyl group as the reactive group, for example, piroxicam and meloxicam. In some forms, the parent drug moiety M′ of the conjugates is the unreacted portion of a NSAID having a carboxyl group and a hydroxyl group, where either group can function as the reactive group, for example, salsalate and diflunisal.

For example, ibuprofen and indomethacin each contain a carboxyl group that can be converted and attach to an alkynyl group, which then reacts with an azide containing aryl group to form the conjugate. In these forms, the parent drug moiety M′ of the conjugate is

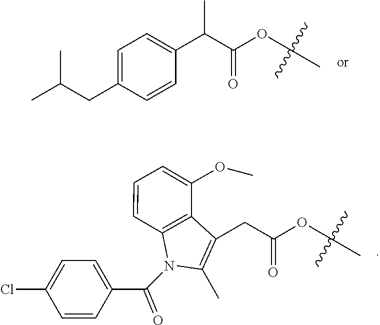 [SMILES: *OC(=O)C(C)c1ccc(CC(C)C)cc1 ; *OC(=O)Cc1c(C)n(C(=O)c2ccc(Cl)cc2)c2cccc(OC)c12]

Similarly, aspirin, mefenamic acid, diclofenac, naproxen, sulindac, salsalate, etodolac, ketoprofen, ketorolac, flurbiprofen, oxaprozin, diflunisal, meclofenamate, fenoprofen, and tolmetin each contain a carboxyl group that can be converted and attach to an alkynyl group, which then reacts with an azide containing aryl group to form the conjugate. In these forms, the parent drug moiety M′ of the conjugate is

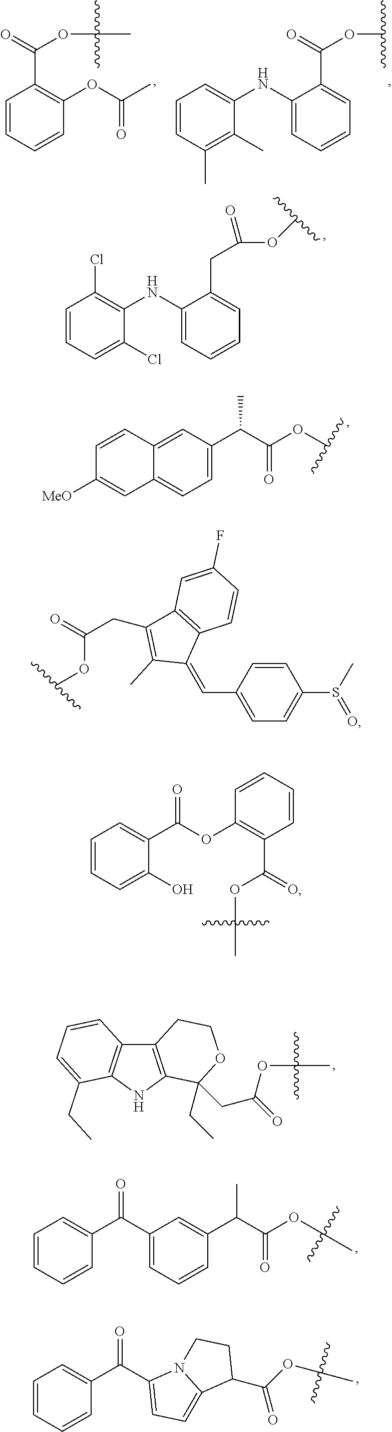

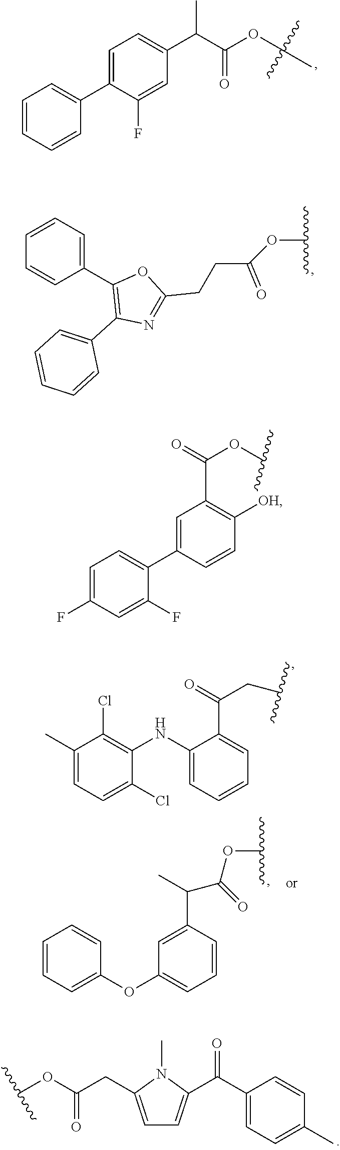 [SMILES: *OC(=O)C(C)c1ccc(-c2ccccc2)c(F)c1 ; *OC(=O)CCc1nc(-c2ccccc2)c(-c2ccccc2)o1 ; *OC(=O)c1cc(-c2ccc(F)cc2F)ccc1O ; *CC(=O)c1ccccc1Nc1c(Cl)ccc(C)c1Cl ; *OC(=O)C(C)c1cccc(Oc2ccccc2)c1 ; *OC(=O)Cc1ccc(C(=O)c2ccc(C)cc2)n1C]

For example, celecoxib contains a primary amino group that can be converted and attach to an alkynyl group, which then reacts with an azide containing aryl group to form the conjugate. In these forms, the parent drug moiety M′ of the conjugate is

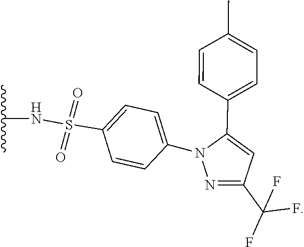 [SMILES: *NS(=O)(=O)c1ccc(-n2nc(C(F)(F)F)cc2-c2ccc(C)cc2)cc1]

For example, piroxicam and meloxicam each contain a hydroxyl group that can be converted and attach to an alkynyl group, which then reacts with an azide containing aryl group to form the conjugate. In these forms, the parent drug moiety M′ of the conjugate is

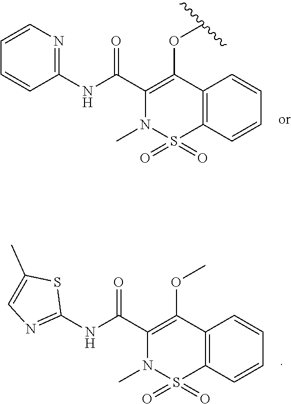 [SMILES: *OC1=C(C(=O)Nc2ccccn2)N(C)S(=O)(=O)c2ccccc21 ; COC1=C(C(=O)Nc2ncc(C)s2)N(C)S(=O)(=O)c2ccccc21]

For example, salsalate and diflunisal each contain a carboxyl group and a hydroxyl group, where either one can be converted and attach to an alkynyl group, which then reacts with an azide containing aryl group to form the conjugate. In these forms, the parent drug moiety M′ of the conjugate is

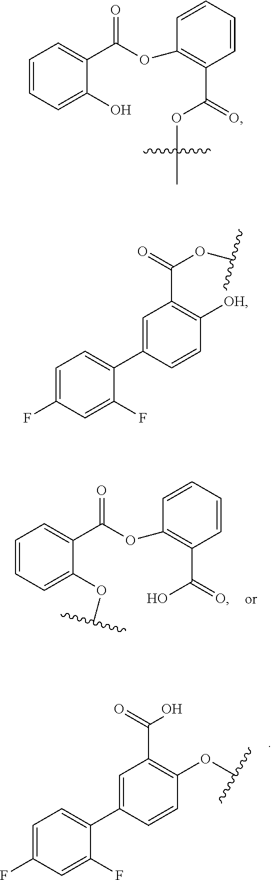 [SMILES: *OC(=O)c1ccccc1OC(=O)c1ccccc1O ; *OC(=O)c1cc(-c2ccc(F)cc2F)ccc1O ; *Oc1ccccc1C(=O)Oc1ccccc1C(=O)O ; *Oc1ccc(-c2ccc(F)cc2F)cc1C(=O)O]

In some forms of Formula I, m of the linker L₁ is an integer from 1 to 4, from 1 to 3, or 1 or 2, such as 1. In some forms of Formula I, R₂ and R₃ of the linker L₁ are independently H or an unsubstituted linear or branched C₁-C₆ alkyl, such as methyl, ethyl, n-propanol, iso-propanol, n-butyl, isobutyl, or tert-butyl. In some forms of Formula I, m of the linker L₁ is 1 or 2, such as 1, and R₂ and R₃ are independently H or an unsubstituted linear or branched C₁-C₄ alkyl, such as methyl, ethyl, n-propanol, iso-propanol, n-butyl, isobutyl, or tert-butyl.

In some forms of Formula I, n is an integer from 1 to 3, or 1 or 2, such as 1. When n is 3, R₁ can be at the ortho- and para-positions, ortho- and meta-positions, or meta- and para-positions. When n is 2, R₁ can be at the ortho-position, meta-position, ortho- and meta-positions, ortho- and para-positions, or meta- and para-positions. When n is 1, R₁ can be at the ortho-position, meta-position, or para-position, preferably ortho-position or para-position.

In some forms of Formula 1, R₁ is a halide, an unsubstituted linear or branched C₁-C₆ alkyl (such as methyl, ethyl, n-propanol, iso-propanol, n-butyl, isobutyl, or tert-butyl), a nitro, or

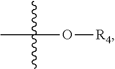

and R₄ is an unsubstituted linear or branched C₁-C₆ alkyl. In some forms of Formula I, R₁ is a halide, an unsubstituted linear or branched C₁-C₄ alkyl, such as methyl, ethyl, n-propanol, iso-propanol, n-butyl, isobutyl, or tert-butyl, a nitro, or

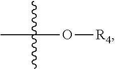

and R₄ is an unsubstituted linear or branched C₁-C₄ alkyl, such as methyl, ethyl, n-propanol, iso-propanol, n-butyl, isobutyl, or tert-butyl. In some forms of Formula I, R₁ is a halide, a methyl or ethyl, a nitro, a methoxy, or an ethoxy.

In some forms of Formula I, n is 1 or 2, R₁ is a halide, an unsubstituted linear or branched C₁-C₆ alkyl (such as methyl, ethyl, n-propanol, iso-propanol, n-butyl, isobutyl, or tert-butyl), a nitro, or

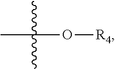

and R₄ is an unsubstituted linear or branched C₁-C₆ alkyl, and R₁ is at the ortho-position, meta-position, ortho- and meta-positions, ortho- and para-positions, or meta- and para-positions.

In some forms of Formula I, n is 1, R₁ is a halide, an unsubstituted linear or branched C₁-C₆ alkyl (such as methyl, ethyl, n-propanol, iso-propanol, n-butyl, isobutyl, or tert-butyl), a nitro, or

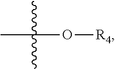

and R₄ is an unsubstituted linear or branched C₁-C₆ alkyl, and R₁ is at the ortho-position or para-position.

The conjugates may contain one or more chiral centers or may otherwise be capable of existing as multiple stereoisomers. These may be pure (single) stereoisomers or mixtures of stereoisomers, such as enantiomers, diastereomers, and enantiomerically or diastereomerically enriched mixtures. The conjugates may be capable of existing as geometric isomers. Accordingly, it is to be understood that the disclosed conjugates include pure geometric isomers or mixtures of geometric isomers.

The conjugates may be neutral or may be one or more pharmaceutically acceptable salts, crystalline forms, non-crystalline forms, hydrates, or solvates, or a combination thereof. References to the conjugates may refer to the neutral molecule, and/or those additional forms thereof collectively and individually from the context. Pharmaceutically acceptable salts of the conjugates include the acid addition and base salts thereof.

Suitable acid addition salts of the conjugates are formed from acids which form non-toxic salts. Examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts of the conjugates are formed from bases which form non-toxic salts. Examples include the aluminum, arginine, benzathine, calcium, choline, diethylamine, diolamine, glycine, lysine, magnesium, meglumine, olamine, potassium, sodium, tromethamine and zinc salts.

Hemisalts of acids and bases of the conjugates may also be formed, for example, hemisulphate and hemicalcium salts.

Exemplary conjugates are shown below:

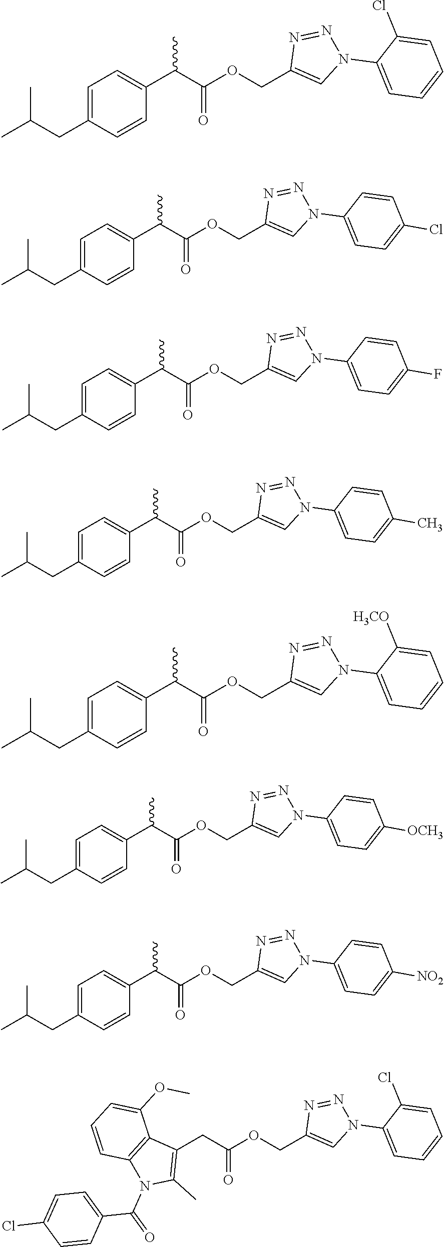

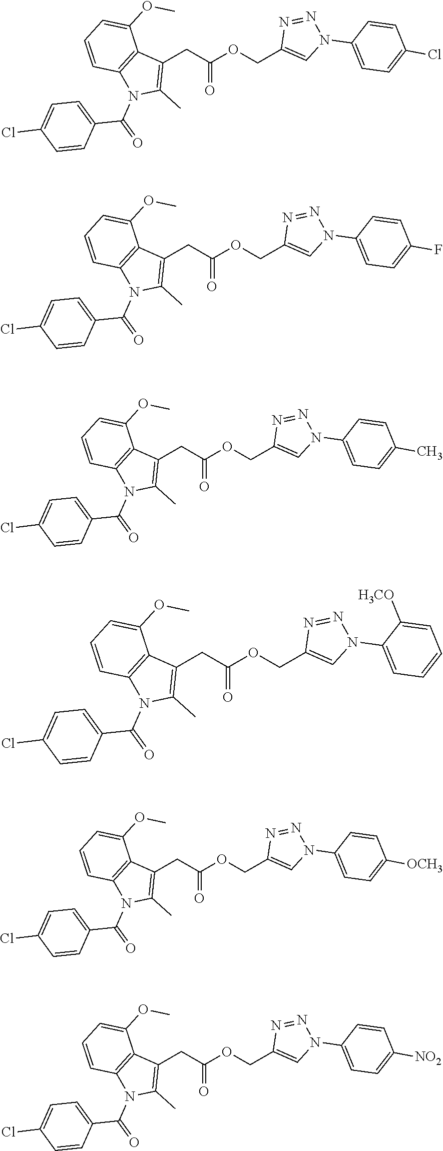 [SMILES: COc1cccc2c1c(CC(=O)OCc1cn(-c3ccc(Cl)cc3)nn1)c(C)n2C(=O)c1ccc(Cl)cc1 ; COc1cccc2c1c(CC(=O)OCc1cn(-c3ccc(F)cc3)nn1)c(C)n2C(=O)c1ccc(Cl)cc1 ; COc1cccc2c1c(CC(=O)OCc1cn(-c3ccc(C)cc3)nn1)c(C)n2C(=O)c1ccc(Cl)cc1 ; COc1ccccc1-n1cc(COC(=O)Cc2c(C)n(C(=O)c3ccc(Cl)cc3)c3cccc(OC)c23)nn1 ; COc1ccc(-n2cc(COC(=O)Cc3c(C)n(C(=O)c4ccc(Cl)cc4)c4cccc(OC)c34)nn2)cc1 ; COc1cccc2c1c(CC(=O)OCc1cn(-c3ccc([N+](=O)[O-])cc3)nn1)c(C)n2C(=O)c1ccc(Cl)cc1]

### B. Pharmaceutical Compositions

Pharmaceutical compositions (also referred herein as “pharmaceutical formulations”) including unit dosage forms suitable for the delivery of the conjugates (including their pharmaceutically acceptable salts) and their preparation are disclosed. Generally, the pharmaceutical formulation contains the conjugates described herein and a pharmaceutically acceptable excipient/carrier. The term “pharmaceutically acceptable carrier” and “pharmaceutically acceptable excipient” are used interchangeably herein to describe any ingredient in the formulation other than the conjugates described herein. The pharmaceutical formulations can include an effective amount of one or more of the conjugates described herein and/or their pharmaceutically acceptable salts (together referred to as “conjugates”), for administration in a subject in need thereof, to prevent an inflammatory disease or disorder, provide analgesia or as an antipyretic. It is to be understood that combinations and/or mixtures of the conjugates may be included in the pharmaceutical composition or formulation.

In some forms, the pharmaceutical composition or formulation can further contain one or more active agents in addition to the conjugates, such as one or more additional anti-inflammatory agents.

Any one or more of the conjugates provided herein can be expressly included or expressly excluded from the pharmaceutical compositions, dosage units, and/or methods of use or treatment disclosed herein.

### 1. Oral Formulations

The conjugates can be administered orally. Oral administration may involve swallowing so that the conjugate enters the gastrointestinal tract, or buccal or sublingual administration may be employed by which the conjugate enters the bloodstream directly from the mouth.

Formulations suitable for oral administration of the conjugates disclosed herein include solid formulations such as tablets, capsules containing particulates, liquids, powders, lozenges (including liquid-filled lozenges), chews, multi- and nano-particulates, gels, solid solutions, liposomes, films, ovules, sprays and liquid formulations. In some forms, the conjugates can be associated with a suitable carrier, such as particles or micelles. e.g. polymeric particles or micelles, lipid particles, and dendrimers. In these forms, the conjugates can be encapsulated in, covalently bond to, and/or complexed with the particles. These particles containing the conjugates can be used for increasing the solubility of the conjugates disclosed herein. Examples of materials suitable for forming the particles (nanoparticles and/or microparticles) containing the disclosed conjugates include, but are not limited to, poly(alkylene glycol) or a copolymer thereof, such as poly(ethylene glycol) (“PEG”) and copolymers thereof, phospholipids, and polyamidoamine (“PAMAM”) or derivatives thereof (e.g. hydroxyl PAMAM). These particles containing the disclosed conjugates may be further formulated into tablets, capsules, powders, etc.

Liquid formulations include suspensions, solutions, syrups and elixirs. Such formulations may be employed as fillers in soft or hard capsules and typically contain a carrier, for example, water, ethanol, polyethylene glycol, propylene glycol, methylcellulose or a suitable oil, and one or more emulsifying agents and/or suspending agents. Liquid formulations may also be prepared by the reconstitution of a solid, for example, from a sachet.

The conjugates may also be used in fast dissolving, fast-disintegrating dosage forms such as those described in Expert Opinion in Therapeutic Patents, 11 (6), 981-986, by Liang and Chen (2001).

For tablet or capsule dosage forms, depending on dose, the conjugates may make up from 1 weight % to 80 weight % of the dosage form or from 5 weight % to 60 weight % of the dosage form. In addition to the conjugates described herein, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will contain from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (as, for example, the monohydrate, spray-dried monohydrate or anhydrous form), mannitol, xylitol, dextrose, sucrose, sorbitol, microcrystalline cellulose, starch and dibasic calcium phosphate dihydrate.

Tablets or capsules may also optionally contain surface active agents, such as sodium lauryl sulfate and polysorbate 80, and glidants such as silicon dioxide and talc. When present, surface active agents may contain from 0.2 weight % to 5 weight % of the tablet, and glidants may contain from 0.2 weight % to 1 weight % of the tablet.

Tablets or capsules also generally contain lubricants such as magnesium stearate, calcium stearate, zinc stearate, sodium stearyl fumarate, and mixtures of magnesium stearate with sodium lauryl sulphate. Lubricants generally contain from 0.25 weight % to 10 weight %, preferably from 0.5 weight % to 3 weight % of the tablet.

Other possible ingredients include glidants (e.g. Talc or colloidal anhydrous silica at about 0.1 weight % to about 3 weight %), antioxidants, colorants, flavouring agents, preservatives and taste-masking agents.

Exemplary tablets contain up to about 80% of one or more of the conjugates described herein, from about 10 weight % to about 90 weight % binder, from about 0 weight % to about 85 weight % diluent, from about 2 weight % to about 10 weight % disintegrant, and from about 0.25 weight % to about 10 weight % lubricant.

Tablet or capsule blends may be compressed directly or by roller to form tablets. Tablet or capsule blends or portions of blends may alternatively be wet-, dry-, or melt-granulated, melt congealed, or extruded before tableting. The final formulation may contain one or more layers and may be coated or uncoated; it may even be encapsulated.

Solid formulations of the conjugates for oral administration may be formulated to be immediate and/or modified release. Modified release formulations include delayed, sustained, pulsed, controlled, targeted and programmed release formulations.

### 2. Parenteral Formulations

The conjugates can also be administered directly into the blood stream, into muscle, or into an internal organ. Suitable routes for such parenteral administration include intravenous, intraarterial, intraperitoneal, intrathecal, epidural, intracerebroventricular, intraurethral, intrasternal, intracranial, intramuscular, and subcutaneous delivery. Suitable means for parenteral administration include needle (including microneedle) injectors, needle-free injectors, and infusion techniques.

Parenteral formulations are typically aqueous solutions which may contain excipients such as salts, carbohydrates and buffering agents (preferably at a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilization, may readily be accomplished using standard pharmaceutical techniques well known to those skilled in the art.

The solubility of the conjugates used in the preparation of a parenteral formulation may be increased by the use of appropriate formulation techniques, such as the incorporation of solubility-enhancing agents or the association of the conjugates with particles, such as those described above. For example, formulations for parenteral administration can contain a suitable carrier that can increase the solubility of the conjugates disclosed herein. For example, the conjugates disclosed herein can be encapsulated in, covalently bond to, or complexed with polymeric nanoparticles, microparticles, or micelles, such as nanoparticles, microparticles, or micelles formed by a poly(lactic-co-glycolic acid), poly(lactic-co-glycolic acid)-poly(ethylene glycol), poly(lactic acid)-poly(ethylene oxide), poly(caprolactone)-poly(ethylene glycol), or a copolymer thereof.

Formulations of the conjugates for parenteral administration may be formulated to be immediate and/or modified release. Modified release formulations include delayed, sustained, pulsed, controlled, targeted and programmed release formulations. Thus, the conjugates may be formulated as a solid, semi-solid, or thixotropic liquid for administration as an implanted depot providing modified release of the active agents. Examples of such formulations include drug-coated stents and poly(dl-lactic-coglycolic)acid (PGLA) microspheres.

### 3. Pulmonary and Mucosal Formulations

The conjugates can be formulated for pulmonary or mucosal administration. The administration can include delivery of the composition to the lungs, nasal, oral (sublingual, buccal), vaginal, or rectal mucosa.

For example, the conjugates can also be administered intranasally or by oral inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atomiser (preferably an atomiser using electrohydrodynamics to produce a fine mist), or nebuliser, with or without the use of a suitable propellant, such as water, ethanol-water mixture, 1,1,1,2-tetrafluoroethane or 1,1,1,2,3,3,3-heptafluoropropane. For intranasal or oral inhalation use, the powder may contain a bioadhesive agent, for example, chitosan or cyclodextrin. The term aerosol as used herein refers to any preparation of a fine mist of particles, which can be in solution or a suspension, whether or not it is produced using a propellant. Aerosols can be produced using standard techniques, such as ultrasonication or high-pressure treatment.

The pressurized container, pump, spray, atomizer, or nebulizer contains a solution or suspension of one or more of the conjugates including, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the conjugates is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or hydroxypropylmethylcellulose), blisters and cartridges for use in an inhaler or insufflator may be formulated to contain a powder mix of the conjugates described herein, a suitable powder base such as lactose or starch and a performance modifier such as 1-leucine, mannitol, or magnesium stearate. The lactose may be anhydrous or in the form of a monohydrate, preferably the latter. Other suitable excipients include dextran, glucose, maltose, sorbitol, xylitol, fructose, sucrose, and trehalose.

A suitable solution formulation for use in an atomizer using electrohydrodynamics to produce a fine mist may contain from 1 μg to 20 mg of one or more of the conjugates described herein per actuation and the actuation volume may vary from 1 μl to 100 μl. A typical formulation may contain one or more of the conjugates described herein, propylene glycol, sterile water, ethanol and sodium chloride. Alternative solvents that may be used instead of propylene glycol include glycerol and polyethylene glycol.

Suitable flavors, such as menthol and levomenthol, or sweeteners, such as saccharin or saccharin sodium, may be added to those formulations intended for inhaled/intranasal administration.

Formulations for inhaled/intranasal administration may be formulated to be immediate and/or modified release using, for example, PGLA. Modified release formulations include delayed, sustained, pulsed, controlled, targeted, and programmed release formulations.

In the case of dry powder inhalers and aerosols, the dosage unit is determined by means of a valve which delivers a metered amount. Units in accordance with the conjugates are typically arranged to administer a metered dose or “puff”. The overall daily dose will be administered in a single dose or, more usually, as divided doses throughout the day.

In some forms, the conjugates described herein can be formulated for pulmonary delivery, such as intranasal administration or oral inhalation. Carriers for pulmonary formulations can be divided into those for dry powder formulations and for administration as solutions. Aerosols for the delivery of therapeutic agents to the respiratory tract are known in the art. For administration via the upper respiratory tract, the formulation can be formulated into an aqueous solution, e.g., water or isotonic saline, buffered or un-buffered, or as an aqueous suspension, for intranasal administration as drops or as a spray. Such aqueous solutions or suspensions may be isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers. One skilled in the art can readily determine a suitable saline content and pH for an innocuous aqueous solution for nasal and/or upper respiratory administration.

In some forms, the aqueous solution is water, physiologically acceptable aqueous solutions containing salts and/or buffers, such as phosphate buffered saline (PBS), or any other aqueous solution acceptable for administration to an animal or human. Such solutions are well known to a person skilled in the art and include, but are not limited to, distilled water, de-ionized water, pure or ultrapure water, saline, phosphate-buffered saline (PBS). Other suitable aqueous vehicles include, but are not limited to. Ringer's solution and isotonic sodium chloride. Aqueous suspensions may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

In some forms, solvents that are low toxicity organic (i.e. nonaqueous) class 3 residual solvents, such as ethanol, acetone, ethyl acetate, tetrahydrofuran, ethyl ether, and propanol may be used for the formulations. The solvent is selected based on its ability to readily aerosolize the formulation. The solvent should not detrimentally react with the conjugates. An appropriate solvent should be used that dissolves the conjugates or forms a suspension of the conjugates. The solvent should be sufficiently volatile to enable formation of an aerosol of the solution or suspension. Additional solvents or aerosolizing agents, such as freons, can be added as desired to increase the volatility of the solution or suspension.

In some forms, the pharmaceutical compositions may contain minor amounts of polymers, surfactants, or other excipients well known to those of the art. In this context, “minor amounts” means no excipients are present that might affect or mediate uptake of the conjugates by cells and that the excipients that are present in amount that do not adversely affect uptake of conjugates by cells. In some forms, the conjugates described herein may be associated with particles, such as those described above. For example, formulations for pulmonary or mucosal administration can contain a suitable carrier that can increase the solubility of the conjugates disclosed herein. For example, the conjugates disclosed herein can be encapsulated in, covalently bond to, or complexed with polymeric nanoparticles, microparticles, or micelles, such as nanoparticles, microparticles, or micelles formed by a poly(alkylene glycol) or a copolymer thereof.

Dry lipid powders can be directly dispersed in ethanol because of their hydrophobic character. For lipids stored in organic solvents such as chloroform, the desired quantity of solution is placed in a vial, and the chloroform is evaporated under a stream of nitrogen to form a dry thin film on the surface of a glass vial. The film swells easily when reconstituted with ethanol. To fully disperse the lipid molecules in the organic solvent, the suspension is sonicated. Nonaqueous suspensions of lipids can also be prepared in absolute ethanol using a reusable PARI LC Jet+ nebulizer (PARI Respiratory Equipment, Monterey, CA).

### 4. Topical Formulations

The conjugates can be administered directly to the external surface of the skin or the mucous membranes (including the surface membranes of the nose, lungs and mouth), such that the conjugates cross the external surface of the skin or mucous membrane and enters the underlying tissues.

Formulations for topical administration generally contain a dermatologically acceptable carrier that is suitable for application to the skin, has good aesthetic properties, is compatible with the active agents and any other components, and will not cause any untoward safety or toxicity concerns.

The carrier can be in a wide variety of forms. For example, emulsion carriers, including, but not limited to, oil-in-water, water-in-oil, water-in-oil-in-water, and oil-in-water-in-silicone emulsions, are useful herein. These emulsions can cover a broad range of viscosities, e.g., from about 100 cps to about 200,000 cps. These emulsions can also be delivered in the form of sprays using either mechanical pump containers or pressurized aerosol containers using conventional propellants. These carriers can also be delivered in the form of a mousse or a transdermal patch. Other suitable topical carriers include anhydrous liquid solvents such as oils, alcohols, and silicones (e.g., mineral oil, ethanol isopropanol, dimethicone, cyclomethicone, and the like); aqueous-based single phase liquid solvents (e.g., hydro-alcoholic solvent systems, such as a mixture of ethanol and/or isopropanol and water); and thickened versions of these anhydrous and aqueous-based single phase solvents (e.g. where the viscosity of the solvent has been increased to form a solid or semi-solid by the addition of appropriate gums, resins, waxes, polymers, salts, and the like). Examples of topical carrier systems useful in the present formulations are described in the following four references all of which are incorporated herein by reference in their entirety: “Sun Products Formulary” Cosmetics & Toiletries, vol. 105, pp. 122-139 (December 1990); “Sun Products Formulary,” Cosmetics & Toiletries, vol. 102, pp. 117-136 (March 1987); U.S. Pat. No. 5,605,894 to Blank et al., and U.S. Pat. No. 5,681,852 to Bissett.

Formulations for topical administration may be formulated to be immediate and/or modified release. Modified release formulations include delayed, sustained, pulsed, controlled, targeted and programmed release formulations. Thus, the conjugates may be formulated as a solid, semi-solid, or thixotropic liquid for administration as an implanted depot providing modified release of the active compound. Examples of such formulations include drug-coated stents and poly(dl-lactic-coglycolic)acid (PGLA) microspheres.

### 5. Additional Active Agent(s)

In some forms, the pharmaceutical composition or pharmaceutical formulation can include one or more additional active agents, such as one or more additional anti-inflammatory agents. Anti-inflammatory agents that can be included in the pharmaceutical compositions or formulations are known, for example, see the WebMD, “Anti-inflammatory Drugs,” web site webmd.com/arthritis/anti-inflammatory-drugs; Barnes, Nature, 402(6760): 31-38 (1999); and Rainsford, Inflammation in the pathogenesis of chronic diseases 3:27 (2007).

Exemplary anti-inflammatory drugs that can be included in the pharmaceutical composition or pharmaceutical formulation include, but are not limited to, ibuprofen, naproxen sodium, aspirin, naproxen sodium, diclofenac potassium, celecoxib, sulindac, oxaprozin, piroxicam, indomethacin, meloxicam, fenoprofen, naproxen, esomeprazole, diclofenac, diflunisal, etodolac, ketorolac tromethamine, katoprofen, meclofenamate, nabumetone, salsalate, tolmetin, and steroids, such as corticosteroids (e.g. hydrocortisone, cortisone, ethamethasoneb, prednisone, prednisolone, triamcinolone, methylprednisolone, and dexamethasone) and mineralocorticoids (e.g. fludrocortisone), and a combination thereof.

### 6. Effective Amount

Effective amounts of the conjugates contained in the pharmaceutical composition or pharmaceutical formulation depend on many factors, including the indication being treated, the route of administration, co-administration of other therapeutic compositions, and the overall condition of the patient. Exemplary effective amount of the conjugates contained in the pharmaceutical formulation (in unit dosage form) can be from 0.01 mg to 1500 mg, from 0.1 mg to 1500 mg, from 1 mg to 1500 mg, from 10 mg to 1500 mg, from 20 mg to 1500 mg, from 0.01 mg to 1000 mg, from 0.1 mg to 1000 mg, from 1 mg to 1000 mg, from 10 mg to 1000 mg, from 20 mg to 1000 mg, from 0.01 mg to 700 mg, from 0.1 mg to 700 mg, from 1 mg to 700 mg, from 10 mg to 700 mg, from 20 mg to 700 mg, from 50 mg to 700 mg, from 0.01 mg to 500 mg, from 0.1 mg to 500 mg, from 1 mg to 500 mg, from 10 mg to 500 mg, from 20 mg to 500 mg, from 50 mg to 500 mg, from 0.01 mg to 100 mg, or from 0.1 mg to 100 mg.

In some forms, the total amount of the one or more conjugates in the pharmaceutical formulation can be at least 0.01 wt %, at least 0.05 wt %, at least 0.1 wt %, in a range from about 0.01 wt % to about 50 wt %, from about 0.05 wt % to about 50 wt %, from about 0.1 wt % to about 50 wt %, from about 0.01 wt % to about 40 wt %, from about 0.05 wt % to about 40 wt %, from about 0.1 wt % to about 40 wt %, from about 0.01 wt % to about 30 wt %, from about 0.05 wt % to about 30 wt %, from about 0.1 wt % to about 30 wt %, from 0.01 wt % to 20 wt %, from about 0.05 wt % to about 20 wt %, from about 0.1 wt % to about 20 wt %, from about 0.1 wt % to about 15 wt %, from about 0.2 wt % to about 20 wt %, from about 0.1 wt % to about 10 wt %, from about 0.5 wt % to about 20 wt %, from about 0.5 wt % to about 15 wt %, from about 0.5 w % to about 10 wt %, from about 0.5 wt % to about 5 wt %, from about 0.1 wt % to about 5 wt %, or from about 0.1 wt % to about 1 wt %. The term “total amount of the one or more conjugates in the pharmaceutical formulation” refers to the sum of the weight of the one or more conjugates relative to the total weight of the pharmaceutical formulation.

### III. Methods of Making the Conjugates

Methods of making the conjugates are disclosed. Generally, the method includes (i) exposing a reaction mixture to a microwave irradiation for a time period sufficient to form a product comprising the conjugates disclosed herein. Typically, the reaction mixture is under stirring during step (i). In some forms, the method produces the conjugates at a yield of at least 60%, at least 65%, at least 70%, or at least 75%. The yield of the conjugate can be calculated using the formula: % yield=(actual weight of the purified conjugate/theoretical weight of the conjugate)×100%.

The reaction mixture contains a first reactant, a second reactant, and a solvent. Suitable solvents for forming the reactant mixture depend on the solubility of the reactants. Typically, the solvent can dissolve all of the reactants and form a solution. Examples of suitable solvents for forming the reaction mixture include, but are not limited to, water, an alcohol, or a mixture thereof, such as a mixture of butanol (n-butanol or tert-butanol) and water. When a mixture of two solvents are used to form the reaction mixture, such as a mixture of butanol and water, the first solvent and second solvent can have a volume ratio in a range from 100:1 to 1:100, from 50:1 to 1:50, from 20:1 to 1:20, from 10:1 to 1:10, from 5:1 to 1:5, or from 2:1 to 1:2, such as 2:1.

In some forms, the reaction mixture can further contain a reducing agent and/or a catalyst. Examples of suitable reducing agents that can be used in the reaction include, but are not limited to, sodium iso-ascorbate, and copper thiophene carboxylate, and a combination thereof. An example of suitable catalysts that can be used in the reaction is CuSO₄.

The first reactant in the reaction mixture can have the structure of Formula II and the second reactant can have the structure of Formula III:

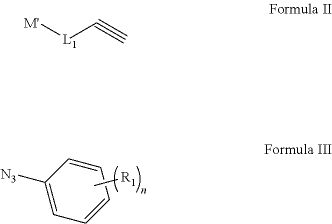

wherein M′, L₁, R₁, and n are as defined above for Formula I.

The microwave irradiation to which the reaction mixture is exposed has an energy suitable for heating the reaction mixture to a temperature suitable for performing the reaction between the first and second reactants. For example, the microwave irradiation has an energy to heat the reaction mixture to a suitable temperature to initiate the reaction and then maintain such a temperature for the reaction mixture for a suitable time period for the reaction to complete. For example, the microwave irradiation used in step (i) has an energy in a range from about 10 W to about 50 W, from about 10 W to about 40 W, from 10 W to about 30 W, or from 15 W to about 25 W, such as about 20 W. Such an energy can heat the reaction mixture to a temperature in a range from about 30° C. to about 100° C., from about 40° C. to about 90° C., or from about 50° C. to about 80° C. such as about 70° C., to initiate reaction between the first and second reactants. This temperature is maintained for the reaction mixture for a suitable period to complete the reaction. For example, the reaction mixture is maintained at a temperature in any of the above-described ranges, for a time period in a range from about 30 mins to about 5 hours, from about 1 hour to about 4 hours, or from about 1 hour to about 3 hours, such as about 2 hours.

Optionally, the method further includes (a) maintaining a second reaction mixture at a suitable temperature for a time period sufficient to form the first reactant. Step (a) is performed prior to step (i).

The second reaction mixture contains a first starting reactant, a second starting reactant, and a solvent. Examples of suitable solvents for forming the second reaction mixture include, but are not limited to, tetrahydrofuran, dichloromethane, dimethyl sulfoxide, dimethyl formamide, ethyl acetate, ethyl lactate, acetone, 1-butanol, 1-propanol, 2-propanol, ethanol, isopropyl acetate, methanol, methyl ethyl ketone, t-butanol, 2-methyl tetrahydrofuran, acetonitrile, or toluene, or a combination thereof.

In some forms, the reaction mixture can further contain a catalyst. Examples of suitable catalysts that can be used in the second reaction mixture include, but are not limited to, Cs₂CO₃, potassium carbonate, triethylamine, sodium bicarbonate, tetrabutylammonium fluoride, and potassium hydroxide, and combinations thereof.

The first starting reactant in the reaction mixture can have the structure of Formula IV and the second starting reactant can have the structure of Formula V:

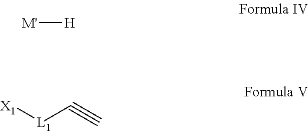

wherein L₁ is as defined above for Formula I and X₁ is a halide, such as fluoride, bromide, or chloride. Formula IV (M′-H) represents the structure of a parent drug, where M′ is the parent drug moiety (i.e., unreacted portion of the parent drug). Following step (a), the H of the parent drug will be replaced by

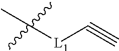

of the second starting reactant of Formula V to form the first reactant of Formula II.

The second reaction mixture can be maintained at a suitable temperature, such as room temperature (20° C. to 25° C. at 1 atm), for a period of time sufficient, such as a time period from 12 hour to 36 hours, to form the first reactant of Formula II. In some forms, the second reaction mixture can be heated from a low temperature, such as about 0° C., to room temperature to initiate the reaction between the first and second starting reactants. The second reaction mixture is then maintained at room temperature for a time period from 12 hour to 36 hours, to form the first reactant of Formula II.

In some forms, the method can further include a step of purifying the product containing the conjugate, such as conjugate having the structure of Formula I, subsequent to step (i). In some forms, the product containing the conjugate can be purified by column chromatography using a suitable organic solvent as eluent, such as ethyl acetate or hexane, or a combination thereof, such as 5% ethyl acetate/95% hexanes. In some forms, following purification, the conjugates is produced in the form of a solid or an oil.

In some forms, the conjugate is a derivative of ibuprofen or indomethacin, which is produced following step (a) and step (i) below and under the conditions as described above.

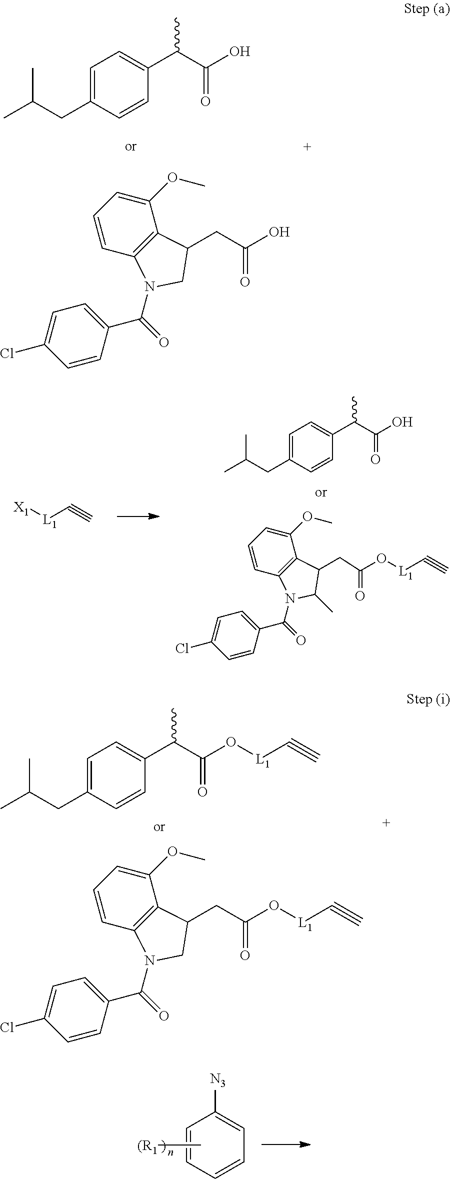

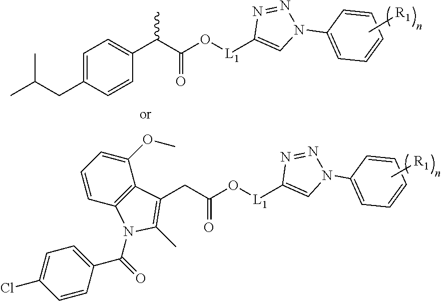

### IV. Methods of Using the Conjugates

Methods of using the conjugates are described herein. The disclosed conjugates can be used for preventing or treating a variety of inflammatory diseases or disorders.

It will be appreciated the disclosed methods can be methods of treatment of the symptoms and conditions associated with inflammatory diseases or disorders. “Treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

Generally, treatment of inflammatory diseases or disorders using the conjugates disclosed herein can have a similar or higher potency/efficacy compared with treatment of the same inflammatory diseases or disorders using their respective parent drugs. For example, treatment of inflammatory diseases or disorders using the conjugates can achieve a similar or higher anti-inflammatory efficiency, as indicated by a similar or more reduction of edema thickness of a body part of a subject in need thereof, compared with a control treated with their respective parent drugs. The conjugates may also have a similar or higher analgesic efficiency, as indicated by a similar or more reduction of peripheral and/or central pain of a subject in need thereof, compared with a control treated with their respective parent drugs.

Further, treatment of inflammatory diseases or disorders using the conjugates disclosed herein can overcome many disadvantages associated with treatment using the current anti-inflammatory drugs, in particular the current NSAIDs. For example, treatment of inflammatory diseases or disorders using the conjugates can achieve a similar or higher anti-inflammatory efficiencies and optionally a similar or higher analgesic efficiencies with no or reduced adverse effects, such as without ulcerogenic effects.

### A. Preventing or Treating Inflammatory Diseases or Disorders

Methods of using the conjugates for preventing or treating an inflammatory disease or disorder in a subject in need thereof are disclosed. The inflammatory disease or disorder can be local or systemic in the subject.

Generally, the method for preventing or treating an inflammatory disease or disorder in a subject in need thereof includes administering to the subject a pharmaceutical formulation containing one or more of the conjugates disclosed herein. The subject can be a mammal, such as a human, an animal kept in a zoo, a domestic animal for example, a dog, a cat, a monkey, rabbits, guinea pigs, etc. or a rat, that is in need of treatment. The administration step can occur one or more times.

Typically, following a single administration or all of the administrations of the pharmaceutical formulation, an effective amount of the conjugates to prevent or treat the inflammatory disease or disorder in the subject, as shown by one or more known clinical and/or biochemical measurements, such as inflammation-associated swelling of a body part (for example as indicated by the edema thickness of the body part) and/or pain score, is administered to the subject. Additional known clinical and biochemical measurements that can be used to evaluate the treatment effect of the pharmaceutical formulation are described in Silvio de Almeida Junior, BrJP., 2(4):386-389 (2019), such as formalin test, formalin-induced orofacial pain, hot plate test, Randall-Selitto test. Von Frey test, Tail flick test. Carrageenan-induced intraplantar edema, Croton oil-induced ear edema in mice, Carrageenan-induced peritonitis, and/or Pleurisy.

In some forms, following a single administration or all of the administrations of the pharmaceutical formulation, the conjugates of the pharmaceutical formulation administered to the subject are in an effective amount to change one or more clinical measurements of the subject, such as to reduce inflammation-associated swelling of a body part and/or reduce or relief pain of the subject, compared with the subject before administered with the pharmaceutical formulation.

For example, following a single administration or all of the administrations of the pharmaceutical formulation, the conjugates of the pharmaceutical formulation administered to the subject are in an effective amount to decrease the edema thickness of a body part of a subject by at least 10%, at least 20%, at least 30%, at least 40%, at least 50%, at least 60%, at least 70%, at least 80%, or at least 90%, compared with the edema thickness of the same body part of the subject before administered with the pharmaceutical formulation; and/or to decrease pain score by at least 10%, at least 20%, at least 40%, at least 50%, at least 60%, at least 70%, at least 80%, or at least 90% of the subject, compared with the subject before administered with the pharmaceutical formulation. The pain may be peripherical or central pain, or a combination thereof.

In some forms, following a single administration or all of the administrations of the pharmaceutical formulation, the conjugates of the pharmaceutical formulation administered to the subject are in an effective amount to change one or more clinical measurements of the subject, such as to reduce inflammation-associated swelling of a body part and/or reduce or relief pain of the subject, compared with the subject before administered with the pharmaceutical formulation, without or with less adverse effects typically associated with current NSAIDs, such as ulcerogenic liability in the gastric.

For example, following a single administration or all of the administrations of the pharmaceutical formulation, the conjugates of the pharmaceutical formulation administered to the subject are in an effective amount to decrease the edema thickness of a body part of a subject by at least 10%, at least 20%, at least 30%, at least 40%, at least 50%, at least 60%, at least 70%, at least 80%, or at least 90%, compared with the edema thickness of the same body part of the subject before administered with the pharmaceutical formulation; and/or to decrease pain score (peripheral and/or central pain) by at least 10%, at least 20%, at least 40%, at least 50%, at least 60%, at least 70%, at least 80%, or at least 90% of the subject, compared with the subject before administered with the pharmaceutical formulation, without ulcerogenic liability in the gastric (such as without developing an ulcer or erosion in the stomach or colon).

In some forms of the method, the conjugates of the pharmaceutical formulation administered to the subject are in an effective amount to reduce or relief inflammation associated swelling of a body part and/or reduce or relief pain of the subject with a similar or higher efficiency, compared with that in a positive control, tested under the same conditions. The “positive control” is a subject having the same inflammatory disease or disorder as the subject treated with the pharmaceutical formulation, but is treated with a current NSAID, such as indomethacin in place of the pharmaceutical formulation disclosed herein.

For example, the conjugates of the pharmaceutical formulation administered to the subject are in an effective amount to reduce edema thickness of a body part and/or to reduce pain score associated with a peripheral or central pain of the subject, compared with the subject before administered with the pharmaceutical formulation, and the level of reduction (for the edema thickness or the pain score, or for each of the edema thickness and pain score) is at least 10%, at least 20%, at least 30%, at least 40%, at least 45%, at least 50%, at least 55%, at least 60%, at least 65%, at least 70%, at least 75%, at least 80%, at least 85%, at least 90%, in a range from 10% to 95%, from 10% to 90%, from 20% to 95%, from 20% to 90%, from 30% to 95%, from 30% to 90%, from 30% to 85%, from 30% to 80%, from 40% to 95%, from 40% to 90%, from 40% to 85%, or from 40% to 80%, more than the level of reduction of edema thickness of the body part and/or the pain score associated with the peripheral or central pain in a positive control, tested under the same conditions.

In some forms, the change of the one or more clinical measurements as described above, such as reduce inflammation associated swelling of a body part of a subject and/or reduce or relief pain of the subject, can occur within 30 mins, within 60 mins, within 2 hours, within 4 hours, within 8 hours, within 12 hours, or within 24 hours following the administration of the pharmaceutical formulation.

For example, the reduction of edema thickness of the body part of the subject as described above can occur within 30 mins, within 1 hour, within 2 hours, within 3 hours, within 4 hours, or within 24 hours, following a single administration or all of the administrations of the pharmaceutical formulation. For example, the reduction or relief of pain of the subject as described above can occur within 30 mins, within 60 mins, within 90 mins, or within 120 mins, following a single administration or all of the administrations of the pharmaceutical formulation.

In some forms, the change of the one or more clinical measurements as described above, such as reduce inflammation associated swelling of a body part of a subject and/or reduce or relief pain of the subject, can occur shortly following the administration of the pharmaceutical formulation, such as within 30 mins, within 60 mins, within 90 mins. or within 120 mins. and last for a time period of at least 2 hours, at least 3 hours, at least 4 hours, at least 6 hours, at least 8 hours, at least 10 hours, at least 12 hours, at least 14 hours, at least 16 hours, at least 18 hours, or at least 24 hours.

For example, the reduction of edema thickness of the body part of the subject as described above can occur within 1 hour, within 2 hours, within 3 hours, or within 4 hours, following a single administration or all of the administrations of the pharmaceutical formulation, and last for at least 3 hours, at least 4 hours, at least 6 hours, at least 8 hours, at least 10 hours, at least 12 hours, at least 16 hours, at least 20 hours, or at least 24 hours.

### 1. Diseases

Both acute and chronic inflammatory diseases or disorders can be treated by the disclosed methods. Examples of suitable inflammatory diseases or disorders and symptoms associated with the inflammatory diseases or disorders that can be treated by the disclosed method include, but are not limited to, asthma, chronic peptic ulcer, tuberculosis, rheumatoid arthritis, periodontitis, ulcerative colitis. Crohn's disease, sinusitis, active hepatitis, acute bronchitis, appendicitis, ingrown toenail, sore throat, and physical trauma or wound, and a combination thereof.

### 2. Administration Routes

The pharmaceutical formulation containing one or more of the disclosed conjugates can be administered to the subject by oral administration, parenteral administration, inhalation, mucosal, topical administration, or a combination thereof.

For example, the pharmaceutical formulation containing one or more of the disclosed conjugates can be orally administered to a subject by a medical professional or the subject being treated (e.g., self-administration). The pharmaceutical formulation containing one or more of the disclosed conjugates can be administered as tablets, capsules containing particulates, granules, powders, lozenges (including liquid-filled lozenges), chews, multi- and nano-particulates, gels, or liquids (e.g., solution or suspensions in aqueous or non-aqueous solvent).

Optionally, the pharmaceutical formulation containing one or more of the disclosed conjugates can be administered to the subject by intravenous injection or intraperitoneal injection. The intravenous injection or intraperitoneal injection can be performed by a medical professional or the subject being treated (e.g., self-injection).

Alternatively, the pharmaceutical formulation containing one or more of the disclosed conjugates can be administered to the subject by inhalation, such as mouth inhalation and/or nasal inhalation.

Optionally, the pharmaceutical formulation containing one or more of the disclosed conjugates can be administered to the subject by topically applying the compound(s) or the pharmaceutical composition or formulation on one or more of the exposed surfaces of the subject.

### 3. Effective Amount

Administering an effective amount of the conjugates of the pharmaceutical formulation can be achieved in a single administration step or using multiple steps of administering the pharmaceutical formulation.

For example, if the unit dosage form contains an effective amount of the conjugates to prevent or treat the inflammatory disease or disorder in the subject, as indicated by one or more clinical measurements and/or one or more biochemical measurements of the subject as described above, then the method only requires a single administration step.

Alternatively, if the unit dosage form contains less than the needed effective amount of the conjugates to prevent or treat the inflammatory disease or disorder in the subject, then the method involves at least two steps of administering the pharmaceutical formulation, and optionally more than two steps of administering the pharmaceutical formulation to the subject until an effective amount of the conjugates is administered to the subject to prevent or treat the inflammatory disease or disorder, as indicated by one or more clinical measurements and/or one or more biochemical measurements of the subject as described above. When multiple administration steps are needed to administer an effective amount of the conjugates to the subject, each administration step may involve administering the same dosage or different dosages of the pharmaceutical formulation to the patient.

In some forms, the method involves a single administration of the pharmaceutical formulation to administer an effective amount of the conjugates to prevent or treat the inflammatory disease or disorder in the subject, as shown by change of one or more clinical measurements and/or one or more biochemical measurements of the subject as described above.

In some forms, treatment regimens utilizing the conjugates include administration of from about 0.1 mg to about 300 mg of the conjugates per kilogram body weight of the recipient per day, achieved in multiple doses or in a single dose. In some embodiments, a suitable dose may be in the range of 0.05 to 300 mg per kilogram body weight of the recipient, 0.05 to 200 mg per kilogram body weight of the recipient, 0.05 to 100 mg per kilogram body weight of the recipient, 0.1 to 300 mg per kilogram body weight of the recipient, 0.1 to 200 mg per kilogram body weight of the recipient, 0.1 to 100 mg per kilogram body weight of the recipient, 1 to 150 mg per kilogram body weight, 1 to 100 mg per kilogram body weight, 2 to 100 mg per kilogram body weight, 2 to 50 mg per kilogram body weight, 2 to 25 mg per kilogram body weight, 5 to 100 mg per kilogram body weight, 5 to 80 mg per kilogram body weight, 5 to 50 mg per kilogram body weight, 5 to 30 mg per kilogram body weight, 0.5 to 50 mg per kilogram body weight, or 5 to 20 mg per kilogram body weight, such as about 10 mg per kilogram body weight. Such a dose may be administered one time or multiple times in a day to achieve a suitable treatment regimen, such as from about 0.1 mg to about 300 mg of the conjugates per kilogram body weight of the recipient per day.

i. Reduction of Nitric Oxide Production

Nitric oxide (“NO”) is a marker of inflammatory responses. Measuring the production of NO can be used for evaluating the progression of inflammation; inhibition of its production can be used for controlling inflammatory reactions and diseases.

In some forms of the method, following a single administration or all of the administrations, the conjugates of the administered pharmaceutical formulation can reduce production of nitric oxide by the macrophages in the subject, compared with the production of nitric oxide by the macrophages in the subject before administered with the pharmaceutical formulation. The production of NO by the macrophages in the subject before and after administered with the pharmaceutical formulation is tested under the same conditions. “Same conditions” for testing the production of nitric oxide means the test is performed using the same assay and using the same protocol, such as the same amount of cells and enzymes, same buffer and buffer concentration, same dye and dye concentration, and same incubation time and temperature, etc.

In some forms of the method, the conjugates of the pharmaceutical formulation administered to the subject are in an effective amount to reduce production of nitric oxide by the macrophages in the subject, compared with the production of nitric oxide by the macrophages in the subject before administered with the pharmaceutical formulation.

In some forms of the method, the conjugates of the pharmaceutical formulation administered to the subject are in an effective amount to reduce the production of NO by the macrophages in the subject by at least 10%, at least 20%, at least 30%, at least 40%, at least 45%, at least 50%, at least 55%, at least 60%, at least 65%, at least 70%, at least 75%, at least 80%, at least 85%, at least 90%, in a range from 10% to 95%, from 10% to 90%, from 20% to 95%, from 20% to 90%, from 30% to 95%, from 30% to 90%, from 30% to 85%, from 30% to 80%, from 40% to 95%, from 40% to 90%, from 40% to 85%, or from 40% to 80%, compared with the production of NO by the macrophages in the subject before administered with the pharmaceutical formulation.

In some forms of the method, the conjugates of the pharmaceutical formulation administered to the subject are in an effective amount to reduce the production of NO by the macrophages, without affecting cell viability of the macrophages in the subject, by at least 10%, at least 20%, at least 30%, at least 40%, at least 45%, at least 50%, at least 55%, at least 60%, at least 65%, at least 70%, at least 75%, at least 80%, at least 85%, at least 90%, in a range from 10% to 95%, from 10% to 90%, from 20% to 95%, from 20% to 90%, from 30% to 95%, from 30% to 90%, from 30% to 85%, from 30% to 80%, from 40% to 95%, from 40% to 90%, from 40% to 85%, or from 40% to 80%, compared with the production of NO by the macrophages in the subject before administered with the pharmaceutical formulation. The term “without affecting cell viability of the macrophages” means that the reduction of cell viability of the macrophages treated with the pharmaceutical formulation is less than about 20%, less than about 15%, less than about 10%, or less than about 5%, in a 24-h period, compared with the cell viability of macrophages in the subject before treated with the pharmaceutical formulation.

In some forms of the method, the conjugates of the pharmaceutical formulation administered to the subject are in an effective amount to reduce production of nitric oxide by the macrophages in the subject, with a similar or higher efficiency compared with the production of nitric oxide by the macrophages in a positive control, tested under the same conditions. The “positive control” is a subject having the same inflammatory disease or disorder as the subject treated with the pharmaceutical formulation, but is treated with a current NSAID, such as indomethacin in place of the pharmaceutical formulation disclosed herein. The reduction of NO production in the positive control is the difference between the amount of NO produced by the macrophage of the subject before treatment and the amount of NO produced by the macrophage of the subject after treatment with the current NSAID.

For example, the conjugates of the pharmaceutical formulation administered to the subject are in an effective amount to reduce the production of NO by the macrophages in the subject compared with the subject before administered with the pharmaceutical formulation, and the reduction is at least 10%, at least 20%, at least 30%, at least 40%, at least 45%, at least 50%, at least 55%, at least 60%, at least 65%, at least 70%, at least 75%, at least 80%, at least 85%, at least 90%, in a range from 10% to 95%, from 10% to 90%, from 20% to 95%, from 20% to 90%, from 30% to 95%, from 30% to 90%, from 30% to 85%, from 30% to 80%, from 40% to 95%, from 40% to 90%, from 40% to 85%, or from 40% to 80%, more than the level of reduction of NO production by the macrophages in a positive control, tested under the same conditions.

ii. Reduction of Proinflammatory Cytokine Release

Macrophages can release inflammatory cytokines, such as IL-10, IL-6, and TNF-α, which participate in the pathogenesis of inflammatory conditions.

In some forms of the method, following a single administration or all of the administrations, the conjugates of the administered pharmaceutical formulation can reduce the amount of an inflammatory cytokine, such as IL-1β, IL-6, and/or TNF-α, released from macrophages of the subject, compared with the amount of the same inflammatory cytokine released from the macrophages in the subject before administered with the pharmaceutical formulation. The amount of released inflammatory cytokine from the macrophages in the subject before and after treatment with the pharmaceutical formulation is determined under the same conditions. “Same conditions” for testing the amount of inflammatory cytokine released from macrophages means that test is performed using the same assay and using the same protocol, such as same amount of cells and enzymes, same buffer and buffer concentration, same dye and dye concentration, and same incubation time and temperature, etc.

In some forms of the method, the conjugates of the pharmaceutical formulation administered to the subject are in an effective amount to reduce the amount of an inflammatory cytokine, such as IL-1P, IL-6, IL-10, and/or TNF-α, released from macrophages in the subject, compared with the amount of the inflammatory cytokine released from the macrophages in the subject before administered with the pharmaceutical formulation, as shown by a reduced mRNA level of the inflammatory cytokine.

In some forms of the method, the conjugates of the pharmaceutical formulation administered to the subject are in an effective amount to reduce the amount of an inflammatory cytokine, such as IL-1, IL-6, and/or TNF-α, released from macrophages in the subject, as shown by a reduced mRNA level of the inflammatory cytokine by at least 5%, at least 10%, at least 15%, at least 20%, at least 25%, at least 30%, at least 35%, at least 40%, at least 45%, at least 50%, in a range from 5% to 70%, from 5% to 60%, from 5% to 50%, from 10% to 70%, from 10% to 60%, from 10% to 50%, from 5% to 45%, from 10% to 45%, from 5% to 40%, or from 10% to 40%, compared with the mRNA levels of the inflammatory cytokine in the subject before administered with the pharmaceutical formulation.

In some forms of the method, the conjugates of the pharmaceutical formulation administered to the subject are in an effective amount to reduce the amount of an inflammatory cytokine, such as IL-1β, IL-6, and/or TNF-α, released from macrophages in the subject, as shown by a reduced mRNA level of the inflammatory cytokine, with a similar or higher efficiency, compared with the amount of the inflammatory cytokine released from macrophages in a positive control, tested under the same conditions. The reduction of inflammatory cytokine release in the positive control is determined as the difference between a mRNA level of the inflammatory cytokine released from the macrophage of the subject before treatment and the mRNA level of the inflammatory cytokine released from the macrophage of the subject after treatment with a current NSAID, such as indomethacin.

For example, the conjugates of the pharmaceutical formulation administered to the subject are in an effective amount to reduce the amount of an inflammatory cytokine, such as IL-10, IL-6, and/or TNF-α, released from macrophages in the subject, with a similar or higher efficiency, compared with the reduction of amount of the inflammatory cytokine released from macrophages in a positive control. The reduction of inflammatory cytokine release with higher efficiency is shown by a more reduced mRNA level of the inflammatory cytokine, compared with the positive control. For example, the reduction of mRNA level of the inflammatory cytokine released from macrophages of the subject is at least 10%, at least 20%, at least 30%, at least 40%, in a range from 10% to 50%, from 10% to 40%, from 20% to 50%, from 20% to 40%, from 10% to 30%, or from 10% to 20%, more than the reduction of the inflammatory cytokine released from the macrophages in a positive control, tested under the same conditions.

In some forms, following a single administration or all of the administrations of the pharmaceutical formulation, the conjugates of the pharmaceutical formulation administered to the subject are in an effective amount to reduce the production of NO by the macrophages in the subject by at least 10%, at least 20%, at least 30%, at least 40%, at least 45%, at least 50%, at least 55%, at least 60%, at least 65%, at least 70%, at least 75%, at least 80%, at least 85%, at least 90%, in a range from 10% to 95%, from 10% to 90%, from 20% to 95%, from 20% to 90%, from 30% to 95%, from 30% to 90%, from 30% to 85%, from 30% to 80%, from 40% to 95%, from 40% to 90%, from 40% to 85%, or from 40% to 80%, compared with the production of NO by the macrophages in the subject before administered with the pharmaceutical formulation; and to reduce the amount of an inflammatory cytokine, such as IL-10, IL-6, and/or TNF-α, released from macrophages in the subject, as shown by a reduced mRNA level of the inflammatory cytokine by at least 5%, at least 10%, at least 15%, at least 20%, at least 25%, at least 30%, at least 35%, at least 40%, at least 45%, at least 50%, in a range from 5% to 70%, from 5% to 60%, from 5% to 50%, from 10% to 70%, from 10% to 60%, from 10% to 50%, from 5% to 45%, from 10% to 45%, from 5% to 40%, or from 10% to 40%, compared with the mRNA levels of the inflammatory cytokine released from the macrophages in the subject before administered with the pharmaceutical formulation.

### 4. Optional Steps

a. Administering Additional Active Agent(s)

One or more active agents (also referred to herein as “additional active agents”) besides the disclosed conjugates may be administered to the subject throughout the method or at different intervals during the method. For example, the one or more additional active agents is administered to the subject prior to, during, and/or subsequent to step (i) administering the pharmaceutical formulation containing the conjugates. In some forms, the one or more additional active agents can be included in the pharmaceutical formulation containing the conjugates and administered to the subject simultaneously with the conjugates.

In some forms, the one or more additional active agents are one or more anti-inflammatory agents as described above. The amount of the one or more additional anti-inflammatory agents administered will vary from subject to the subject according to their need The disclosed compounds, pharmaceutical compositions, methods of making, and methods of using can be further understood through the following numbered paragraphs.
1. A compound having the structure of:

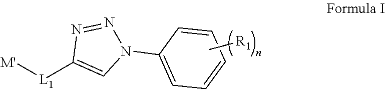

wherein:
(i) R₁ is a halide, an unsubstituted alkyl, a substituted alkyl, an alkoxy, a nitro, a carbonyl, a hydroxyl, an amino, an amido, a cyano, a thio, an unsubstituted alkenyl, a substituted alkenyl, an unsubstituted alkynyl, a substituted alkynyl, an unsubstituted alkylaryl, a substituted alkylaryl, an unsubstituted aryl, or a substituted aryl;
(ii) n is an integer from 0 to 5, from 0 to 4, from 0 to 3, or 1 or 2, such as 1;

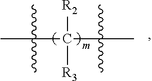

(iii) L₁ is R₃,
   wherein m is an integer from 0 to 6,
   R₂ and R₃ are independently H, an unsubstituted alkyl, or a substituted alkyl;
(iv) M′ is a parent drug moiety; and
(v) each substituent is independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a carbonyl, an alkoxy, a halogen, a hydroxyl, a phenoxy, a thiol, an alkylthio, a phenylthio, an arylthio, a cyano, an isocyano, a nitro, a carboxyl, an amino, an amido, an oxo, a silyl, a sulfinyl, a sulfonyl, a sulfonic acid, a phosphonium, a phosphanyl, a phosphoryl, or a phosphonyl.
2. The compound of paragraph 1, wherein M′ is

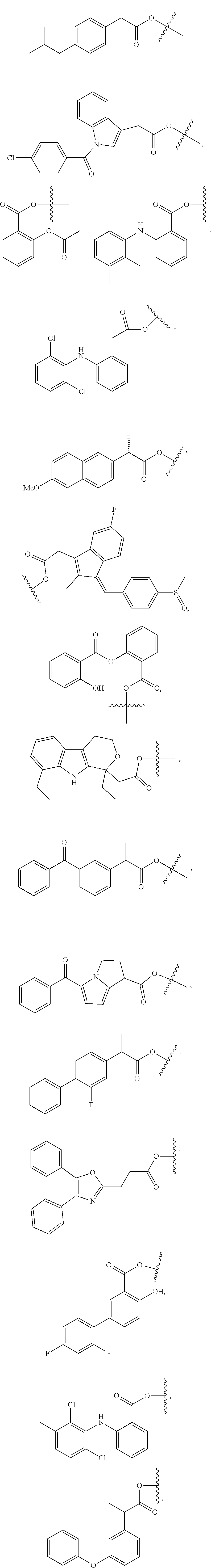

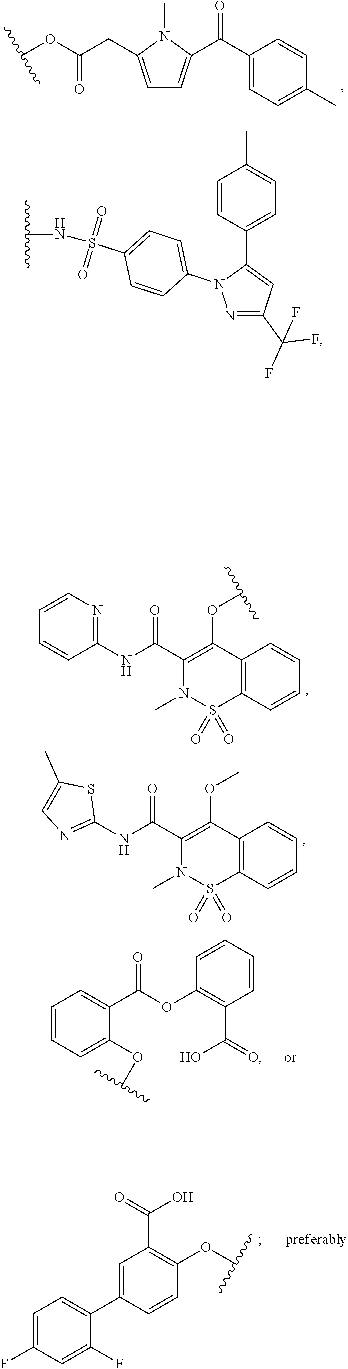

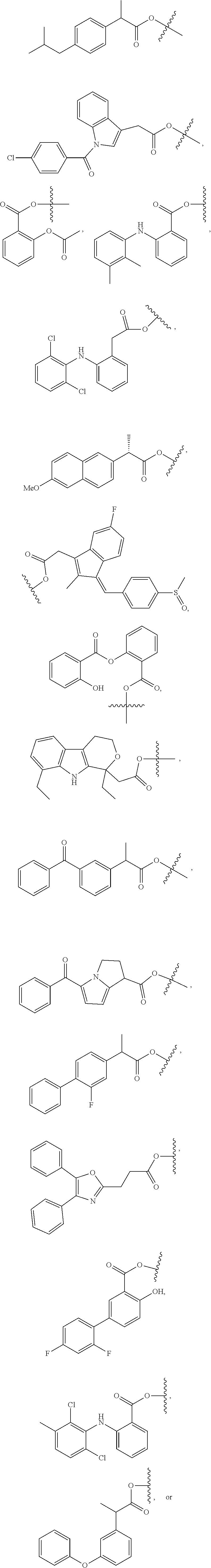

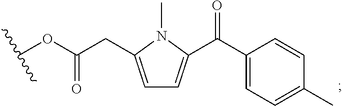 [SMILES: *OC(=O)Cc1ccc(C(=O)c2ccc(C)cc2)n1C]

more preferably or

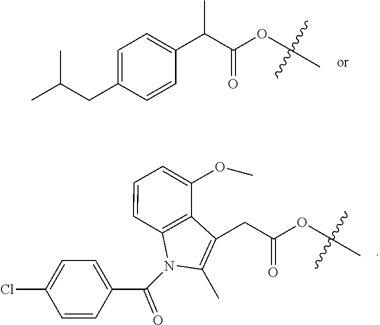 [SMILES: *OC(=O)C(C)c1ccc(CC(C)C)cc1 ; *OC(=O)Cc1c(C)n(C(=O)c2ccc(Cl)cc2)c2cccc(OC)c12]

3. The compound of paragraph 1 or 2, wherein m is an integer from 1 to 4, and R₂ and R₃ are independently H or an unsubstituted linear or branched C₁-C₆ alkyl, such as methyl, ethyl, 1-propanol, isopropanol, 1-butyl, isobutyl, or tertbutyl.
4. The compound of paragraph 3, wherein m is 1 or 2, and R₂ and R₃ are independently H or methyl.
5. The compound of any one of paragraphs 1-4, wherein n is an integer from 1-3, such as 1.
6. The compound of any one of paragraphs 1-5, wherein R₁ is a halide, an unsubstituted linear or branched C₁-C₆ alkyl, a nitro, or

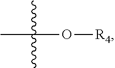

wherein R₄ is an unsubstituted linear or branched C₁-C₆ alkyl.
7. The compound of any one of paragraphs 1-6, wherein n is 1 and R₁ is at the ortho- or para-position.
8. The compound of any one of paragraphs 1-7, wherein the compound has the structure of.

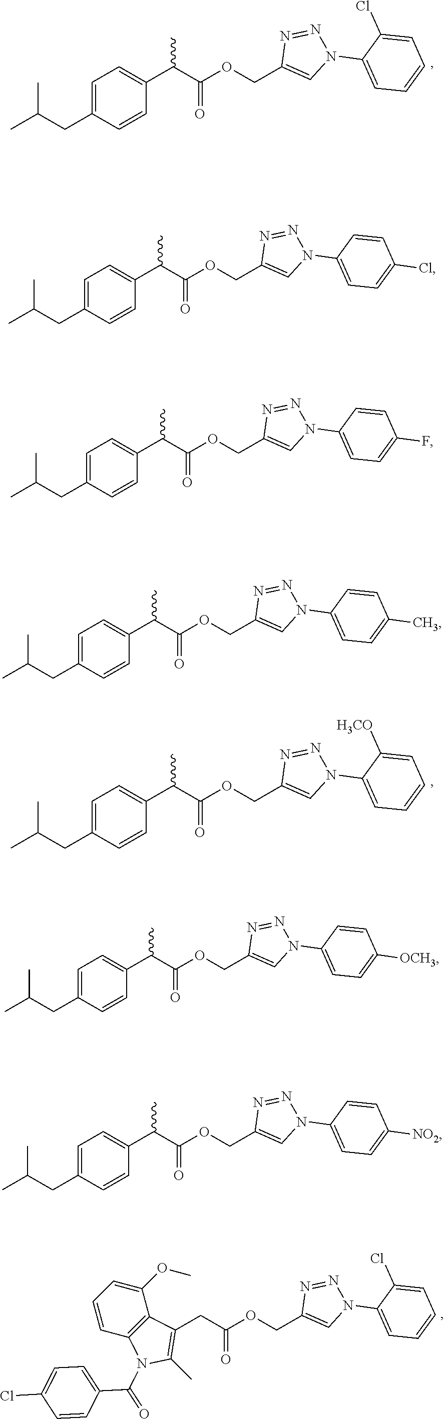

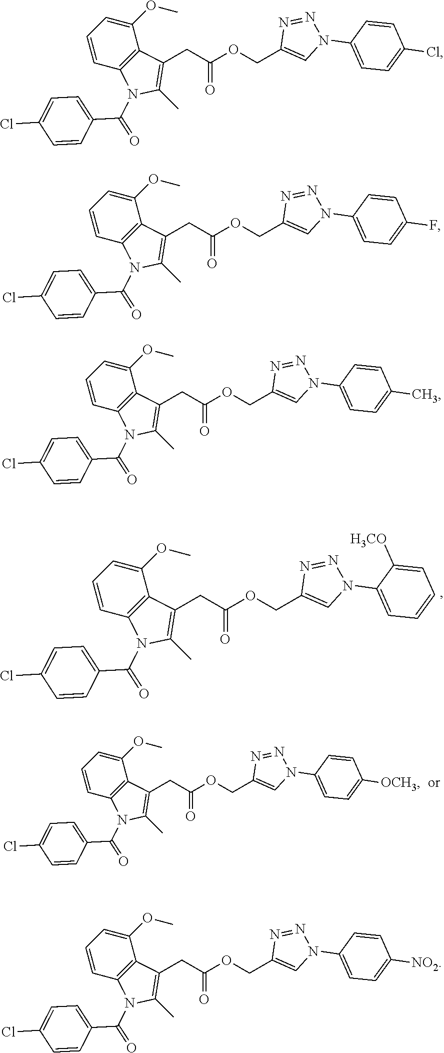

9. A pharmaceutical formulation comprising one or more compounds of any one of paragraphs 1-8; and a pharmaceutically acceptable excipient,
   wherein the one or more compounds are in an effective amount to prevent or treat an inflammatory disease or disorder in a subject.
10. The pharmaceutical formulation of paragraph 9, wherein the pharmaceutical formulation further comprises one or more active agents, and optionally wherein the one or more active agents is/are one or more anti-inflammatory agent.
11. The pharmaceutical formulation of paragraph 9 or 10, wherein the total amount of the one or more compounds in the pharmaceutical formulation is at least 0.01 wt %, at least 0.05 wt %, at least 0.1 wt %, in a range from 0.01 wt % to 50 wt %, from 0.05 wt % to 50 wt %, from 0.1 wt % to 50 wt %, from 0.01 wt % to 40 wt %, from 0.05 wt % to 40 wt %, from 0.1 wt % to 40 wt %, from 0.01 wt % to 30 w t %, from 0.05 wt % to 30 wt %, from 0.1 wt % to 30 wt %, from 0.01 wt % to 20 wt %, from 0.05 wt % to 20 wt %, from 0.1 wt % to 20 wt %, from 0.01 wt % to 10 wt %, from 0.05 wt % to 10 wt %, or from 0.1 wt % to 10 wt %.
12. A method of producing the compound of any one of paragraphs 1-8 comprising:
   (i) exposing a first reaction mixture to a microwave irradiation for a time period sufficient to form a product comprising the compound,
   wherein the first reaction mixture comprises a first reactant, a second reactant, and a first solvent,
   wherein the first reactant has the structure of:

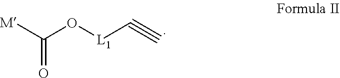

wherein the second reactant has the structure of:

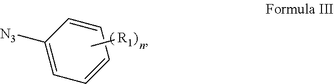

wherein:
(i) R₁ is a halide, an unsubstituted alkyl, a substituted alkyl, an alkoxy, a nitro, a carbonyl, a hydroxyl, an amino, an amido, a cyano, a thio, an unsubstituted alkenyl, a substituted alkenyl, an unsubstituted alkynyl, a substituted alkynyl, an unsubstituted alkylaryl, a substituted alkylaryl, an unsubstituted aryl, or a substituted aryl;
(ii) n is an integer from 0 to 5, from 0 to 4, from 0 to 3, or 1 or 2, such as 1;
(iii) L₁ is

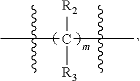

wherein m is an integer from 0 to 6,
R₂ and R₃ are independently H, an unsubstituted alkyl, or a substituted alkyl;
(iv) M′ is a parent drug moiety; and
(v) each substituent is independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a carbonyl, an alkoxy, a halogen, a hydroxyl, a phenoxy, a thiol, an alkylthio, a phenylthio, an arylthio, a cyano, an isocyano, a nitro, a carboxyl, an amino, an amido, an oxo, a silyl, a sulfinyl, a sulfonyl, a sulfonic acid, a phosphonium, a phosphanyl, a phosphoryl, or a phosphonyl.
13. The method of paragraph 12, wherein the microwave irradiation has an energy in a range from about 10 W to about 50 W, from about 10 W to about 40 W, from 10 W to about 30 W, or from 15 W to about 25 W, such as about 20 W.
14. The method of paragraph 12 or 13, wherein the first reaction mixture is maintained at a temperature in a range from about 30° C. to about 100° C., from about 40° C. to about 90° C., or from about 50° C. to about 80° C., such as about 70° C. for a time period in a range from about 30 mins to about 5 hours, from about 1 hour to about 4 hours, or from about 1 hour to about 3 hours, such as about 2 hours.
15. The method of any one of paragraphs 12-14, wherein the first solvent is water, an alcohol, or a mixture thereof, such as a mixture of 1-butanol and water.
16. The method of any one of paragraphs 12-15, wherein the reaction mixture further comprises a reducing agent and/or a first catalyst, and optionally wherein the reducing agent is sodium iso-ascorbate and/or copper thiophene carboxylate and the first catalyst is CuSO₄.
17. The method of any one of paragraphs 12-16, wherein the reaction mixture is under stirring during step (i).
18. The method of any one of paragraphs 12-17, wherein the method further comprises:
   (a) maintaining a second reaction mixture at a suitable temperature for a time period sufficient to produce the first reactant,
   wherein step (a) is performed prior to step (i),
   wherein the second reaction mixture comprises a first starting reactant, a second starting reactant, and a second solvent,
   wherein the first starting reactant has the structure of:

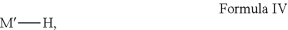

wherein the second starting reactant has the structure of:

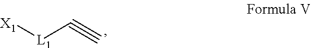

wherein:
(i) M′ and L₁ are as defined above in paragraph 12; and
(ii) X₁ is a halide, such as fluoride, bromide, or chloride.
19. The method of paragraph 18, wherein step (a) was maintained at room temperature (20° C. to 25° C. at 1 atm) for a time period in a range from 12 hour to 36 hours.
20. The method of paragraph 19, wherein prior to step (a), the second reaction mixture is heated from about 0° C. to the room temperature.
21. The method of any one of paragraphs 18-20, wherein the second solvent is tetrahydrofuran, dichloromethane, dimethyl sulfoxide, dimethyl formamide, ethyl acetate, ethyl lactate, acetone, 1-butanol, 1-propanol, 2-propanol, ethanol, isopropyl acetate, methanol, methyl ethyl ketone, t-butanol, 2-methyl tetrahydrofuran, acetonitrile, or toluene, or a combination thereof
22. The method of any one of paragraphs 18-21, wherein the second reaction mixture further comprises a second catalyst, and optionally wherein the second catalyst is Cs₂CO₃, potassium carbonate, triethylamine, sodium bicarbonate, tetrabutylammonium fluoride, or potassium hydroxide, or combinations thereof
23. The method of any one of paragraphs 12-22, further comprising purifying the product containing the compound subsequent to step (i).
24. The method of any one of paragraphs 12-23, wherein the compound has a yield of at least 60%, at least 65%, at least 70%, or at least 75%.
25. A method for preventing or treating an inflammatory disease or disorder in a subject in need thereof comprising
   (i) administering to the subject the pharmaceutical formulation of any one of paragraphs 9-11, wherein step (i) occurs one or more times.
26. The method of paragraph 25, wherein the method comprises only a single administration of the pharmaceutical formulation, wherein the pharmaceutical formulation comprises an effective amount of the compounds to change one or more clinical and/or biochemical measurements associated with the inflammatory disease or disorder of the subject (such as reduce or relief inflammation associated swelling and/or reduce or relief pain), compared to the subject before administered with the pharmaceutical formulation.
27. The method of paragraph 25, wherein the method comprises more than one step of administering to the subject the pharmaceutical formulation, wherein following all of the administration steps, an effective amount of the compounds to change one or more clinical and/or biochemical measurements associated with the inflammatory disease or disorder of the subject (such as reduce or relief inflammation associated swelling and/or reduce or relief pain), compared to the subject before administered with the pharmaceutical formulation, is administered to the subject.
28. The method of paragraph 26 or 27, wherein the effective amount of the compound is effective to reduce or relief inflammation associated swelling, as indicated by reduction of the edema thickness of a body part of the subject by at least 10%, at least 20%, at least 30%, at least 50%, at least 60%, at least 70%, at least 80%, or at least 90% within about 24 hours, about 12 hours, about 10 hours, about 5 hours, about 4 hours, about 3 hours, about 2 hours, or about 1 hour following the single administration or all of the administrations of the pharmaceutical formulation.
29. The method of any one of paragraphs 26-28, wherein the effective amount of the compound is effective to reduce or relief pain associated with the inflammatory disease or disorder.
30. The method of any one of paragraphs 26-29, wherein following the administration or all of the administrations of the pharmaceutical formulation, the subject does not develop an ulcer or erosion in the gastrointestinal system.
31. The method of any one of paragraphs 25-30, wherein in step (i), the pharmaceutical formulation is administered by oral administration, intramuscular administration, intravenous administration, intraperitoneal administration, or subcutaneous administration, or a combination thereof.
32. The method of any one of paragraphs 25-31, wherein during step (i), the dosage of the compounds in the pharmaceutical formulation is from about 0.1 mg to about 100 mg, from about 0.5 mg to about 50 mg, from about 1 mg to about 100 mg, from about 2 mg to about 100 mg, from about 2 mg to about 50 mg, from about 2 mg to about 25 mg, or from about 5 mg to about 20 mg per kg of the subject, such as about 10 mg per kg of the subject.
33. A method for treating macrophages in a subject in need thereof comprising:
   (i) administering to the subject the pharmaceutical formulation of any one of paragraphs 9-11, wherein step (i) occurs one or more times.
34. The method of paragraph 33, wherein the method comprises only a single administration of the pharmaceutical formulation, wherein the pharmaceutical formulation comprises an effective amount of the compounds to reduce production of nitric oxide by macrophages and/or reduce release of one or more inflammatory cytokines from the macrophages, compared with the subject before administered with the pharmaceutical formulation.
35. The method of paragraph 33, wherein the method comprises more than one step of administering to the subject the pharmaceutical formulation, wherein following all of the administration steps, an effective amount of the compounds to reduce production of nitric oxide by macrophages and/or reduce release of one or more inflammatory cytokines from the macrophages is administered to the subject, compared with the subject before administered with the pharmaceutical formulation.
36. The method of paragraph 34 or 35, wherein the reduction of the release of the one or more inflammatory cytokines is indicated by a decrease in the mRNA level associated with the inflammatory cytokine or each of the inflammatory cytokines.
37. The method of any one of paragraphs 34-36, wherein the inflammatory cytokine is or each of the inflammatory cytokines independently is IL-6, TNF-α, or iNOS.

### EXAMPLES

### Example 1: Exemplary Conjugates Show Anti-Inflammatory and Analgesic Properties, without Ulcerogenic Liability

### Materials and Methods

### Chemistry

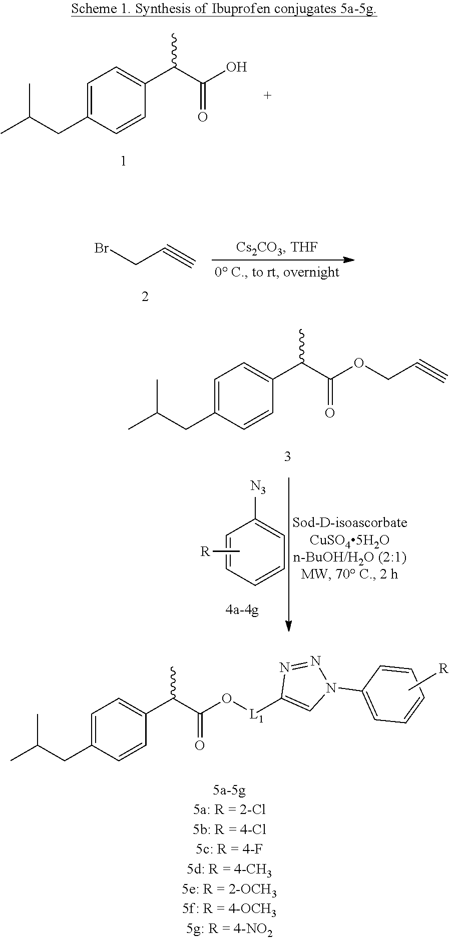

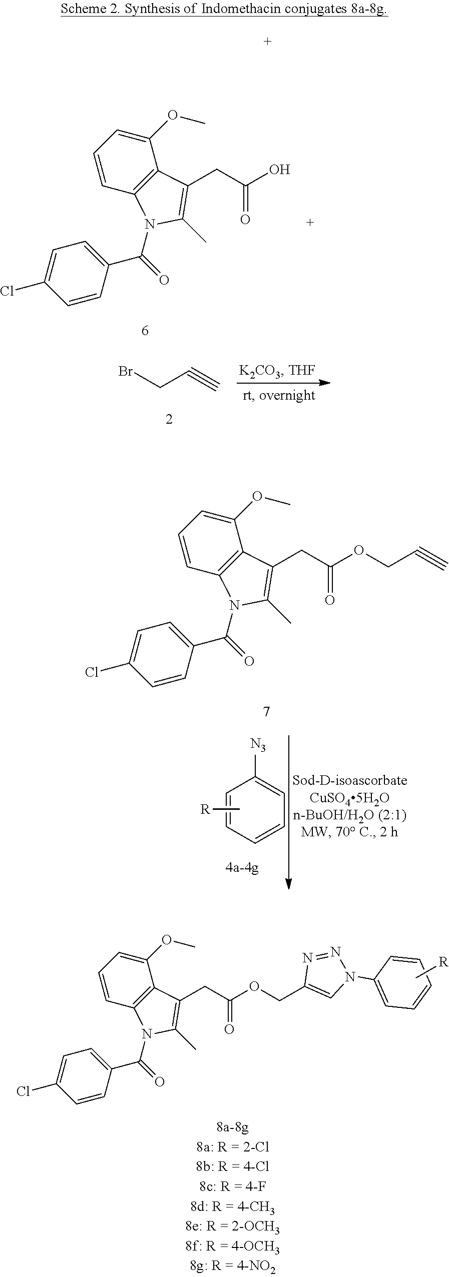

### General Method for Preparing 3 and 7

Melting points were determined on a capillary point apparatus equipped with a digital thermometer and were uncorrected. NMR spectra were recorded in CDCl₃ on a Bruker spectrometer operating at 500 MHz for ¹H NMR (with TMS as an internal standard) and 125 MHz for ¹³C NMR. The microwave-assisted reactions were performed with a single-mode cavity Discover Microwave Synthesizer (CEM Corporation, NC). The reaction mixtures were transferred into a 10 mL glass pressure microwave tube equipped with a magnetic stir bar. The tube was closed with a silicon septum and the reaction mixture was subjected to microwave irradiation (Discover mode; run time: 120 s; Power Max-cooling mode). High-resolution mass spectra were recorded with a TOF analyzer spectrometer using electron spray mode.

### Preparation of prop-2-yn-1-yl 2-(4-isobutylphenyl) propanoate (3)

To a solution of ibuprofen 1 (100 mg, 0.48 mmol) in THF (5 mL), cesium carbonate (316 mg, 0.97 mmol) and propargyl bromide 2 (0.10 mL, 0.97 mmol) were added. The reaction mixture was stirred starting at 0° C., raised to room temperature for reaction overnight. TLC was used to monitor the progress of the reaction. After completion of the reaction, the solvent was evaporated under reduced pressure and the residue was treated with cold water, extracted with ethyl acetate, and then dried under vacuum to get the desired compound 3 in pure form. Colorless oil, yield: 99% (117 mg). IR: νₘₐₓ/cm⁻¹; 3046, 2954, 2869, 2200, 1739, 1512, 1198; ¹H NMR δ: 7.18 (d, J=8.1 Hz, 2H, Ar—H), 7.08 (d, J=8.1 Hz, 2H, Ar—H), 4.69 (dd, J=15.6, 2.5 Hz, 1H, CH₂), 4.58 (dd, J=15.6, 2.5 Hz, 1H, CH₂), 3.72 (q, J=7.2 Hz, 1H, CH), 2.44-2.41 (m, 3H, CH+CH₂), 1.87-1.79 (m, 1H, CH), 1.49 (d, J=7.2 Hz, 3H, CH₃), 0.88 (d, J=6.6 Hz, 6H, 2CH₃); ¹³C NMR δ: 174.1, 140.9, 137.4, 129.6, 127.4, 77.9, 75.0, 52.4, 45.2, 45.1, 30.4, 22.6, 18.8; HRMS: m/z for C₁₆H₂₀O₂ [M⁺] Calcd.: 244.1463, Found: 244.1460.

### Preparation of prop-2-yn-1-yl 2-(1-(4-chlorobenzoyl)-5-methoxy-2-methyl-1H-indol-3-yl) acetate (7)

In a round bottom flask (50 mL) containing a small stir bar, a suspension of indomethacin 6 (500 mg, 1.40 mmol) in DMF (20 mL) along with anhydrous K₂CO₃ (386 mg, 2.79 mmol) were added. The reaction mixture was stirred at room temperature for 30 min then propargyl bromide 2 (0.25 mL, 2.79 mmol) was added. The reaction mixture was stirred overnight, and TLC was used to monitor the progress of the reaction. After completion of the reaction, the reaction mixture was poured on iced water and extracted with ethyl acetate (20 mL) three times. The combined organic layer was dried over sodium sulfate. The crude product was subjected to column chromatography to give pure compound 7. Brown oil, yield: 97% (1.07 g). IR: νₘₐₓ/cm⁻¹; 3032, 2931, 2900, 2120, 1739, 1656, 1478, 1255, 835: ¹H NMR δ: 7.63 (d, J=8.4 Hz, 2H, Ar—H), 7.44 (d, J=8.4 Hz, 2H, Ar—H), 6.94 (d, J=2.5 Hz, 1H. Ar—H), 6.85 (d, J=9.0 Hz, 1H, Ar—H), 6.65 (dd, J=9.0, 2.4 Hz, 1H, Ar—H), 4.68 (d, J=2.4 Hz, 2H, CH₂), 3.81 (s, 3H, OCH₃), 3.69 (s, 2H, CH₂), 2.45 (s, 1H, CH), 2.36 (s, 3H, CH₃); ¹³C NMR δ: 170.1, 168.4, 162.7, 156.2, 139.4, 136.2, 134.0, 131.3, 130.9, 130.6, 129.3, 115.1, 111.9, 101.3, 77.6, 75.3, 55.8, 52.6, 36.6, 31.6, 30.2, 13.5; HRMS: m/z for C₂₂H₁₈ClNO₄ [M⁺] Calcd.: 395.0924, Found: 395.0931.

General Method for Preparation of Compounds 5a-5g and 8a-5g:

In a dried heavy-walled Pyrex tube containing a small stir bar, a solution of the respective alkyne derivative (either 3 or 7) (500 mg, 1.0 eq.) in in n-BuOH/H₂O mixture (for 3) or t-BuOH/H₂O mixture (for 7) (2:1, 3 mL) were added. Sodium D-isoascorbate monohydrate (0.4 eq.) and copper sulfate pentahydrate (0.3 eq.) were added at room temperature. The corresponding aryl azide 4a-4g (1.2 eq.) was added. The reaction mixture was exposed to microwave irradiation (20 W) at 70° C. for 2 h, monitored by TLC. The mixture was allowed to cool down and then quenched with ice-cold water (15 mL). The product was extracted with ethyl acetate. The organic layer was washed with brine solution and dried over anhydrous sodium sulfate. The solvent was removed under reduced pressure and the targeted compounds 5a-5g and 8a-8g were isolated in good yields after purification using column chromatography (10% ethyl acetate/hexanes).

### (1-(2-Chlorophenyl)-1H-1,2,3-triazol-4-yl)methyl 2-(4-isobutylphenyl)propanoate (5a)

Yellow oil, yield: 79% (0.64 g). IR: νₘₐₓ/cm⁻¹; 3018, 2954, 2868, 1732, 1496, 1235, 1199, 759; ¹H NMR δ: 7.80 (s, 1H, CH), 7.55-7.53 (m, 2H, Ar—H), 7.45-7.39 (m, 2H, Ar—H), 7.31 (d, J=8.1 Hz, 2H, Ar—H), 7.04 (d, J=8.1 Hz, 2H, Ar—H), 5.31 (s, 2H, CH₂), 3.73 (q, J=7.2 Hz, 1H, CH), 2.38 (d, J=7.2 Hz, 2H, CH₂), 1.81-1.75 (m, 1H, CH), 1.48 (d, J=7.2 Hz, 3H, CH₃), 0.84 (d, J=6.6 Hz, 6H, 2CH₃); ¹³C NMR δ: 178.6, 174.7, 143.1, 140.8, 137.5, 134.9, 131.0, 130.9, 128.8, 128.1, 127.9, 127.4, 127.3, 125.7, 58.0, 45.2, 30.3, 22.5, 18.5; HRMS: m/z for C₂₂H₂₄ClN₃O₂ [M⁺] Calcd.: 397.1557, Found: 397.1563.

### (1-(4-Chlorophenyl)-1H-1,2,3-triazol-4-yl)methyl 2-(4-isobutylphenyl)propanoate (5b)

Yellowish white solid, m.p. 92-94° C., yield: 86% (0.70 g). IR: νₘₐₓ/cm⁻¹; 3046, 2972, 2882, 1734, 1503, 1231, 1197, 822; ¹H NMR δ: 7.73 (s, 1H, CH), 7.60-7.57 (m, 2H, Ar—H), 7.47-7.45 (m, 2H, Ar—H), 7.17 (d, J=8.0 Hz, 2H, Ar—H), 7.06 (d, J=8.0 Hz, 2H, Ar—H), 5.28 (s, 2H, CH₂), 3.72 (q. J=7.1 Hz, 1H, CH), 2.40 (d, J=7.2 Hz, 2H, CH₂), 1.83-1.75 (m, 1H, CH), 1.48 (d, J=7.2 Hz, 3H, CH₃), 0.85 (d, J=6.6 Hz, 6H, 2CH₃); ¹³C NMR δ: 174.8, 144.3, 141.0, 137.5, 135.5, 134.9, 130.1, 129.6, 127.4, 121.9, 121.6, 58.1, 45.2, 30.4, 22.6, 18.5; HRMS: m/z for C₂₂H₂₄ClN₃O₂ [M⁺] Calcd.: 397.1557, Found: 397.1559.

### (1-(4-Fluorophenyl)-1H-1,2,3-triazol-4-yl)methyl 2-(4-isobutylphenyl)propanoate (5c)

Yellowish white solid, m.p. 80-82° C., yield: 85% (0.66 g). IR: νₘₐₓ/cm⁻¹, 3098, 2954, 1728, 1514, 1229, 1200, 1053; ¹H NMR δ: 7.71 (s, 1H, CH), 7.63-7.59 (m, 2H, Ar—H), 7.18 (t, J=8.3 Hz, 4H, Ar—H), 7.06 (d, J=8.0 Hz, 2H, Ar—H), 5.28 (s, 2H, CH₂), 3.72 (q, J=7.1 Hz, 1H, CH), 2.40 (d, J=7.2 Hz, 2H, CH₂), 1.84-1.75 (m, 1H, CH), 1.48 (d, J=7.2 Hz, 3H, CH₃), 0.85 (d, J=6.6 Hz, 6H, 2CH₃); ¹³C NMR δ: 174.8, 163.7, 161.7, 144.2, 140.9, 137.5, 133.3, 129.6, 127.4, 122.8, 121.9, 117.0, 116.8, 58.1, 45.2, 30.4, 22.6, 18.5; HRMS: m/z for C₂₂H₂₄FN₃O₂ [M⁺] Calcd: 381.1853, Found: 381.1859.

### (1-(p-Tolyl)-1H-1,2,3-triazol-4-yl)methyl 2-(4-isobutylphenyl)propanoate (5d)

Yellowish white solid, m.p. 84-86° C., yield: 92% (0.71 g). IR: νₘₐₓ/cm⁻¹; 3016, 2973, 2926, 2868, 1735, 1520, 1232, 1198; ¹H NMR δ: 7.73 (s, 1H, CH), 7.51 (d, J=8.4 Hz, 2H, Ar—H), 7.27 (d, J=8.2 Hz, 2H, Ar—H), 7.17 (d, J=8.0 Hz, 2H, Ar—H), 7.06 (d, J=8.1 Hz, 2H, Ar—H), 5.28 (s, 2H, CH₂), 3.72 (q, J=7.1 Hz, 1H, CH), 2.41 (s, 2H, CH₂), 2.40 (s, 3H, CH₃), 1.84-1.76 (m, 1H, CH), 1.48 (d, J=7.2 Hz, 3H, CH₃), 0.85 (d, J=6.6 Hz, 6H, 2CH₃); ¹³C NMR δ: 174.8, 143.8, 140.9, 139.2, 137.5, 134.8, 130.4, 129.5, 127.4, 121.7, 120.6, 58.2, 45.2, 30.3, 22.6, 21.3, 18.5; HRMS: m/z for C₂₃H₂₇N₃O₂ [M⁺] Calcd.: 377.2103, Found: 377.2110.

### (1-(2-Methoxyphenyl)-1H-1,2,3-triazol-4-yl)methyl 2-(4-isobutylphenyl)propanoate (5e)

Yellow oil, yield: 87% (0.62 g). IR: νₘₐₓ/cm⁻¹; 3048, 2954, 2868, 1732, 1602, 1506, 1254, 1198; ¹H NMR δ: 8.00 (s, 1H, CH), 7.72 (dd, J=7.9, 1.6 Hz, 1H, Ar—H), 7.40 (dt. J=8.3, 1.6 Hz, 1H, Ar—H), 7.17 (d, J=8.0 Hz, 2H, Ar—H), 7.09-7.04 (m, 4H, Ar—H), 5.33 (d, J=12.8 Hz, 1H, CH₂), 5.25 (d, J=12.8 Hz, 1H, CH₂), 3.84 (s, 3H, OCH₃), 3.72 (t, J=7.2 Hz, 1H, CH), 2.39 (d, J=7.2 Hz, 2H, CH₂), 1.83-1.75 (m, 1H, CH), 1.48 (d, J=7.2 Hz, 3H, CH₃), 0.85 (d, J=6.6 Hz, 6H, 2CH₃); ¹³C NMR δ: 174.8, 151.3, 142.5, 140.8, 137.6, 130.4, 129.5, 127.4, 126.0, 125.7, 121.4, 112.4, 58.3, 56.1, 45.2, 30.4, 22.6, 18.7; HRMS: m/z for C₂₃H₂₇N₃O₃ [M⁺] Calcd.: 393.2052, Found: 393.2059.

### (1-(4-Methoxyphenyl)-1H-1,2,3-triazol-4-yl)methyl 2-(4-isobutylphenyl)propanoate (5j)

Colorless oil, yield: 85% (0.68 g), IR: νₘₐₓ/cm⁻¹; 3132, 2955, 2867, 2843, 1727, 1518, 1440; ¹H NMR δ: 7.73 (s, 1H, CH), 7.55 (d, J=9.0 Hz, 2H, Ar—H), 7.21 (d, J=8.1 Hz, 2H, Ar—H), 7.08 (d, J=8.1 Hz, 2H, Ar—H), 6.99 (d, J=9.0 Hz, 2H, Ar—H), 5.30 (s, 2H, OCH₂), 3.85 (s, 3H, OCH₃), 3.75 (q, J=7.2 Hz, 1H), 2.43 (d, J=7.2 Hz, 2H, CH₂), 1.87-1.78 (m, 1H, CH), 1.51 (d, J=7.2 Hz, 3H, CH₃), 0.88 (d, J=6.6 Hz, 6H, 2CH₃); ¹³C NMR δ: 174.6, 159.9, 143.6, 140.7, 137.4, 130.3, 129.4, 127.2, 122.1, 121.7, 114.8, 56.0, 55.6, 45.0, 44.9, 30.2, 22.4, 18.3; HRMS: m/z for C₂₃H₂₇N₃O₃ [M]⁺ Calcd.: 393.2052. Found: 393.2055.

### (1-(4-Nitrophenyl)-1H-1,2,3-triazol-4-yl)methyl 2-(4-isobutylphenyl)propanoate (5g)

Yellow crystals, yield: 89% (0.74 g), m.p. 65° C. IR: νₘₐₓ/cm⁻¹; 3139, 3098, 2954, 2866, 1740, 1598, 1517, 1371; ¹H NMR δ: 8.43 (d, J=9.1 Hz, 2H, Ar—H), 7.92 (d, J=9.1 Hz, 2H, Ar—H), 7.88 (s, 1H, CH), 7.23 (d, J=8.0 Hz, 2H, Ar—H), 7.11 (d, J=8.0 Hz, 2H, Ar—H), 5.38-5.32 (m, 2H, OCH₂), 3.78 (q, J=7.1 Hz, 1H, CH), 2.46 (d, J=7.2 Hz, 2H, CH₂), 1.89-1.81 (m, 1H, CH), 1.54 (d, J=7.1 Hz, 3H, CH₃), 0.90 (d, J=6.6 Hz, 6H, 2CH₃); ¹³C NMR δ: 174.6, 147.3, 144.8, 141.0, 140.8, 137.2, 129.4, 127.3, 125.5, 121.4, 120.5, 57.7, 45.0, 44.9, 30.2, 22.4, 18.2; HRMS: m/z for C₂₂H₂₄N₄O₄ [M]⁺ Calcd.: 408.1797. Found: 408.1801.

### (1-(2-Chlorophenyl)-1H-1,2,3-triazol-4-yl)methyl 2-(1-(4-chlorobenzoyl)-5-methoxy-2-methyl-H-indol-3-yl)acetate (8a)

Yellow oil, yield: 65% (0.45 g). IR: νₘₐₓ/cm⁻¹; 3045, 2948, 2879, 1737, 1680, 1590, 1477, 1316, 1221, 729; ¹H NMR δ: 7.92 (s, 1H, CH), 7.62 (d, J=8.5 Hz, 2H, Ar—H), 7.54 (dd, J=6.9, 2.4 Hz, 2H, Ar—H), 7.46-7.42 (m, 4H, Ar—H), 6.90 (d, J=2.5 Hz, 1H, Ar—H), 6.85 (d, J=9.0 Hz, 1H, Ar—H), 6.63 (dd, J=9.0, 2.5 Hz, 1H, Ar—H), 5.34 (s, 2H, CH₂), 3.76 (s, 3H, OCH₃), 3.70 (s, 2H, CH₂), 2.33 (s, 3H, CH₃); ¹³C NMR δ: 170.9, 168.5, 156.3, 142.7, 139.5, 136.2, 134.8, 134.1, 131.4, 131.1, 131.0, 130.7, 129.3, 128.8, 128.1, 127.9, 126.1, 115.2, 112.4, 111.9, 101.4, 58.3, 55.9, 30.5, 13.6; HRMS: m/z for C₂₈H₂₂Cl₂N₄O₄ [M⁺] Calcd.: 548.1018, Found: 548.1011.

### (1-(4-Chlorophenyl)-1H-1,2,3-triazol-4-yl)methyl 2-(1-(4-chlorobenzoyl)-5-methoxy-2-methyl-1H-indol-3-yl)acetate (8b)

Yellow oil, yield: 87% (0.59 g), IR: νₘₐₓ/cm⁻¹; 3152, 3112, 2924, 2851, 1735, 1677, 1309; ¹H NMR δ: 7.85 (s, 1H, CH), 7.66 (d, J=8.4 Hz, 2H, Ar—H), 7.62 (d, J=8.8 Hz, 2H, Ar—H), 7.51 (d, J=8.8 Hz, 2H, Ar—H), 7.49 (d, J=8.4 Hz, 2H, Ar—H), 6.96 (d, J=2.5 Hz, 1H, Ar—H), 6.91 (d, J=9.0 Hz, 1H, Ar—H), 6.69 (dd, J=9.0, 2.5 Hz, 1H, Ar—H), 5.36 (s, 2H, OCH₂), 3.80 (s, 3H, OCH₃), 3.75 (s, 2H, CH₂), 2.39 (s, 3H, CH₃); ¹³C NMR δ: 170.7, 168.3, 156.0, 143.8, 139.4, 136.1, 135.3, 134.7, 133.8, 131.2, 130.8, 130.5, 130.0, 129.2, 121.7, 115.0, 112.1, 111.6, 101.4, 58.1, 55.7, 30.3, 13.4; HRMS: m/z for C₂₈H₂₂Cl₂N₄O₄ [M]⁺ Calcd.: 548.1018. Found: 548.1021.

### (1-(4-Fluorophenyl)-1H-1,2,3-triazol-4-yl)methyl 2-(1-(4-chlorobenzoyl)-5-methoxy-2-methyl-1H-indol-3-yl)acetate (8c)

White crystals, yield: 85% (0.57 g), m.p. 71° C. IR: νₘₐₓ/cm⁻¹; 3093, 2925, 2853, 1731, 1688, 1525, 1477; ¹H NMR δ: 7.84 (s, 1H, CH), 7.67-7.63 (m, 4H, Ar—H), 7.49 (d, J=8.5 Hz, 2H, Ar—H), 7.25-7.22 (m, 2H, Ar—H), 6.96 (d, J=2.5 Hz, 1H, Ar—H), 6.91 (d, J=9.0 Hz, 1H, Ar—H), 6.69 (dd, J=9.0, 2.5 Hz, 1H, Ar—H), 5.36 (s, 2H, CH₂O), 3.79 (s, 3H, OCH₃), 3.74 (s, 2H, CH₂), 2.39 (s, 3H, CH₃); ¹³C NMR δ: 170.7, 168.3, 162.5 (d, J=249.4 Hz), 156.1, 143.6, 139.4, 136.1, 133.8, 133.1, 131.2, 130.8, 130.5, 129.2, 122.5 (d, J=8.6 Hz), 122.0, 116.8 (d, J=23.2 Hz), 115.0, 112.1, 111.6, 101.4, 58.2, 55.7, 30.3, 13.4; HRMS: m/z for C₂₈H₂₂ClFN₄O₄ [M]⁺ Calcd.: 532.1313. Found: 532.1321.

### (1-(p-Tolyl)-1H-1,2,3-triazol-4-yl)methyl 2-(1-(4-chlorobenzoyl)-5-methoxy-2-methyl-1H-indol-3-yl)acetate (8d)

Yellow solid, m.p. 130-132° C., yield: 72% (0.48 g). IR: νₘₐₓ/cm⁻¹; 3002, 2954, 2918, 1725, 1673, 1608, 1519, 1332, 1216, 818; ¹H NMR δ: 7.80 (s, 1H, CH), 7.61 (d, J=8.5 Hz, 2H, Ar—H), 7.49 (d, 0.1=8.4 Hz, 2H, Ar—H), 7.42 (d, 0.1=8.5 Hz, 2H, Ar—H), 7.28 (d, J=8.2 Hz, 2H, Ar—H), 6.92 (d, J=2.5 Hz, 1H, Ar—H), 6.86 (d, J=9.0 Hz, 1H, Ar—H), 6.64 (dd, J=9.0, 2.5 Hz, 1H, Ar—H), 5.32 (s, 2H, CH₂), 3.75 (s, 3H, OCH₃), 3.70 (s, 2H, CH₂), 2.40 (s, 3H, CH₃), 2.34 (s, 3H, CH₃); ¹³C NMR δ: 170.9, 168.5, 156.3, 143.5, 139.5, 139.3, 136.3, 134.7, 134.0, 131.4, 131.0, 130.7, 130.5, 129.3, 122.0, 120.6, 115.2, 112.4, 111.9, 101.5, 58.4, 55.9, 30.5, 21.3, 13.6; HRMS: m/z for C₂₉H₂₅ClN₄O₄ [M⁺] Calcd.: 528.1564, Found: 528.1571.

### (1-(2-Methoxyphenyl)-1H-1,2,3-triazol-4-yl)methyl 2-(1-(4-chlorobenzoyl)-5-methoxy-2-methyl-1H-indol-3-yl)acetate (8e)

Yellow paste, yield: 63% (0.43 g). IR: νₘₐₓ/cm⁻¹; 3061, 2987, 1729, 1681, 1589, 1532, 1311, 1217, 740; ¹H NMR δ: 8.24 (s, 1H, CH), 8.07 (d, J=8.8 Hz, 1H, Ar—H), 7.99 (dd, J=8.8, 2.3 Hz, 1H, Ar—H), 7.93 (d, J=2.2 Hz, 1H, Ar—H), 7.62-7.60 (m, 2H, Ar—H), 7.44-7.43 (m, 2H, Ar—H), 6.88-6.84 (m, 3H, Ar—H), 6.61 (dd, J=9.0, 2.5 Hz, 1H, Ar—H), 5.33 (s, 2H, CH₂), 3.97 (s, 3H, OCH₃), 3.74 (s, 3H, OCH₃), 3.69 (s, 2H, CH₂), 2.33 (s, 3H, CH₃); ¹³C NMR δ: 170.9, 168.5, 156.2, 150.8, 148.4, 142.9, 139.6, 136.2, 134.0, 131.4, 131.0, 130.9, 130.7, 129.3, 126.0, 125.4, 116.9, 115.1, 112.3, 111.9, 108.0, 101.5, 58.2, 57.0, 55.9, 30.4, 13.6; HRMS: m/z for C₂₉H₂₅ClN₄O₅ [M⁺] Calcd.: 544.1513, Found: 544.1511.

### (1-(4-Methoxyphenyl)-1H-1,2,3-triazol-4-yl)methyl 2-(1-(4-chlorobenzoyl)-5-methoxy-2-methyl-1H-indol-3-yl)acetate (8l)

Brown solid, m.p. 108-110° C. yield: 67% (0.46 g). IR: νₘₐₓ/cm⁻¹; 3004, 2926, 1726, 1645, 1606, 1519, 1215, 831; H NMR δ: 7.77 (s, 1H, CH), 7.60 (d, J=8.5 Hz, 2H, Ar—H), 7.51 (d, J=9.0 Hz, 2H, Ar—H), 7.42 (d, J=8.5 Hz, 2H, Ar—H), 6.97 (d, J=9.0 Hz, 2H, Ar—H), 6.91 (d, J=2.5 Hz, 1H, Ar—H), 6.86 (d, J=9.0 Hz, 1H, Ar—H), 6.64 (dd, J=9.0, 2.5 Hz, 1H, Ar—H), 5.31 (s, 2H, CH₂), 3.84 (s, 3H, OCH₃), 3.74 (s, 3H, OCH₃), 3.69 (s, 2H, CH₂), 2.33 (s, 3H, CH₂); ¹³C NMR δ: 170.9, 168.5, 160.2, 156.2, 143.4, 139.5, 136.2, 134.0, 131.3, 131.0, 130.7, 130.4, 129.3, 122.3, 122.2, 115.2, 115.0, 112.4, 111.9, 101.5, 58.3, 55.8, 30.5, 13.6; HRMS: m/z for C₂₉H₂₅ClN₄O₅ [M⁺] Calcd.: 544.1513, Found: 544.1519.

### (1-(4-Nitrophenyl)-1H-1,2,3-triazol-4-yl)methyl 2-(1-(4-chlorobenzoyl)-5-methoxy-2-methyl-1H-indol-3-yl)acetate (8g)

Yellow crystals, yield: 85% (0.60 g), m.p. 67° C. IR: νₘₐₓ/cm⁻¹; 3093, 2925, 2853, 1731, 1688, 1596, 1525; ¹H NMR δ: 8.42 (d, J=9.1 Hz, 2H, ArH), 7.93 (s, 1H, Ar—H), 7.89 (d, J=9.1 Hz, 2H, Ar—H), 7.67 (d, J=8.5 Hz, 2H, Ar—H), 7.49 (d, J=8.5 Hz, 2H, Ar—H), 6.97 (d, J=2.5 Hz, 1H, Ar—H), 6.92 (d, J=9.0 Hz, 1H, Ar—H), 6.70 (dd. J=9.0, 2.5 Hz, 1H, Ar—H), 5.39 (s, 2H, CH₂O), 3.80 (s, 3H, OCH₃), 3.76 (s, 2H, CH₂), 2.40 (s, 3H, CH₃); ¹³C NMR δ: 170.7, 168.4, 156.0, 147.4, 144.5, 140.9, 139.5, 136.2, 133.7, 131.2, 130.9, 130.4, 129.2, 125.5, 121.6, 120.5, 115.0, 112.0, 111.6, 101.5, 58.0, 55.8, 30.3, 13.4; HRMS: m/z for C₂₈H₂₂ClN₅O₆ [M]⁺ Calcd.: 559.1258. Found: 559.1255.

### Biological and Computational Studies

Details of the experimental techniques utilized for biological and computational studies are described below. All the biological procedures followed the standards and were approved by the Research Ethics Committee, Faculty of Pharmacy, Cairo University, Egypt (number PC: 2989). All the experiments were performed in accordance with the relevant guidelines and regulations.

### Anti-Inflammatory Activity Screening

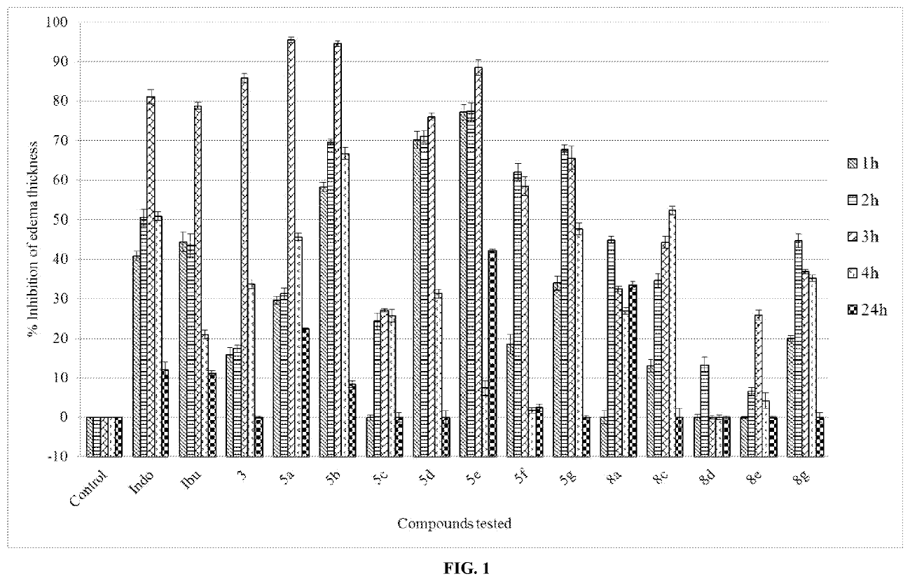

The anti-inflammatory activity of the conjugates was determined in-vivo by the acute carrageenan induced paw edema standard method in rats. Adult Wister rats of either sex (pregnant female animals were excluded) weighing 120-150 g were divided into 19 groups of 6 animals each. The conjugates were dissolved in DMSO, at a dose of 10 mg kg⁻¹ (rat body weight) indomethacin mol equivalent, and were given to rats in test groups intraperitoneally 1 h before induction of inflammation. The control group was given DMSO only. Paw edema was induced by subcutaneous injection of freshly prepared 1% solution of carrageenan in saline (0.9%, 0.1 ml per rat) into sub plantar tissue of the right hind paw of rats. The thickness of the paw was measured (in mm) after successive time intervals (1, 2, 3, 4 and 24 h) and compared with the initial hind pav thickness of each rat for determining the edema thickness. Data were collected, checked, corrected, and analyzed (SPSS 16). Quantitative variables from normal distribution were expressed as means±SE “standard error”. The anti-inflammatory activity was expressed as percentage inhibition of edema thickness in treated animals in comparison with the control group according to eqn. (1) (Table 1, FIG. 1).

$$\% Inhibition of edema = \frac{\text{?}}{\text{?}} \times 100 \qquad ( 1 )$$

$$\text{?} \text{indicates text missing or illegible when filed}$$

where, V_{c} and Vₜ are the means of edema paw thickness for the control and tested compound treated animal groups, respectively.

The potency of the tested conjugates was expressed as % inhibition of edema thickness for the tested compounds relative to % inhibition of edema for indomethacin “reference standard” at 3 h effect “indomethacin reveals its maximum bio-properties at the mentioned time” according to eqn. (2) (Table 1).

$$\% Potency = \frac{\begin{matrix}\% Inhibition of edema for the tested compound \\ treated group\end{matrix}}{\% Inhibition of edema for indomethacin treated group} \times 100 \qquad ( 2 )$$

### Peripheral Analgesic Testing

Peripheral analgesic activity was measured by the standard acetic acid-induced writhing test in mice. Six albino mice of either sex (20-25 g) were used in each group (19 groups). Mice were intraperitoneally (i,p.) administered with 10 mg kg⁻¹ (mice body weight) indomethacin mol equivalent of the conjugates, ibuprofen, or indomethacin suspended in distilled/sterile water with two drops of Tween 80. One hour after the i.p. administration, each mouse was injected with 0.1 ml of 1% acetic acid solution i.p. The control group animals were given sterile/distilled water with few drops of Tween 80. Starting 5 min after the acetic acid injection, the number of muscular contractions in each mouse was counted for 30 min. A significant reduction in the number of writhing by a conjugate compared with control animals was considered as a positive analgesic response. Percentage protection was calculated according to equ. (3), where n is the average number of writhing in the control group and n′ is the average number of writhing in the treated group. The % potency was calculated by equ. (4) (Table 2).

$$\% Inhibition / protection = \frac{n - {n}^{′}}{n} \times 100 \qquad ( 3 )$$

$$\% Potency = \frac{\% protection of the tested compound}{\% protection of indomethacin} \times 100 \qquad ( 4 )$$

### Central Analgesic (Hot Plate) Testing

An in vivo standard hot plate test was performed. Six albino mice of either sex (20-25 g) were used in each group (19 groups). Mice were intraperitoneally (i.p.) administered with 10 mg kg⁻¹ (mice body weight) indomethacin mol equivalent of the tested compounds and reference standards (indomethacin and ibuprofen) suspended in sterile/distilled water with two drops of Tween 80. After the i.p. administration, mice were screened by placing them on a hot plate maintained at 55±1° C. for 30 min (LSI Letica hot plate LE-7406) and recording the reaction time in seconds for forepaw licking or jumping. The control group animals were given sterile/distilled water with few drops of Tween 80. The maximum cutoff time was 15 seconds to prevent tissue damage. Response latencies were measured at 30, 60, 90 and 120 inns. The % protection was calculated by equ. (5), where, Ti, To are the latency time mean of the tested compound and control group respectively (Table 3).

$$\% Protection = \frac{{T}_{1} - {T}_{0}}{{T}_{0}} \times 100 \qquad ( 5 )$$

### Ulcerogenic Liability

The ulcerogenic liability of conjugates was determined in albino mice following a standard method. Animals of either sex (pregnant females were excluded) weighing 20-25 g were divided into 8 groups of 6 animals each. The animals fasted 18 h before drug administration. Conjugates 3, 5a, 5b, 5d, and 5e and the reference standard (indomethacin and ibuprofen) were suspended in distilled water with a few drops of Tween 80. The suspensions were administered orally for three successive days to fasted animals at a dose of 10 mg kg¹ (animal body weight) indomethacin mol equivalent. The control group animals were given water with a few drops of Tween 80. The animals were sacrificed by cervical dislocation and the stomach was removed, opened along the greater curvature, and rinsed with saline. The gastric mucosa was examined with a magnifying lens (10×) for the presence of lesions and erosions. The ulcer index was calculated (Table 4) and the degree of ulcerogenic effect was expressed in terms of: (1) the percentage incidence of ulcers divided by 10; (2) average number of ulcers per stomach; and (3) average severity of ulcers. The ulcer index is the value that resulted from the sum of the above three values.

### Toxicological Bioassay

Toxicological study of conjugates 3, 5a, 5b, 5d, 5e was determined utilizing a standard method in mice. Albino mice weighing 20-25 g were divided into 6 groups of 6 mice each. The conjugates were dissolved in distilled water with a few drops of Tween 80. Then the liquid was given intraperitoneally to each mice in 50 mg kg⁻¹ (mice body weight) indomethacin mol equivalent (i.e. 5 folds of the anti-inflammatory dosage). The control group was given water with a few drops of Tween 80. The toxic symptoms and mortality rates were recorded 24 h post-administration in each group.

### Inhibition of COX-1 and COX-2

The inhibitory properties of conjugates 3, 5a, 5b, 5d, and 5e against COX-1 and COX-2 were determined using a standard technique following the manufacturer's instructions.

### Measurement of Nitrite Concentration Using Griess Method

The Griess method was used to determine the nitrite concentration in cell culture medium. RAW264.7 macrophages were seeded at a density of 1×10⁶ cells/mL in a 96-well plate for 2 h. Experimental data were collected for normal control, LPS-stimulated cells only (10 ng/mL), and LPS-stimulated cells treated with conjugates 5a, 5b, 5d, and 5e respectively. LPS-stimulated RAW264.7 macrophages were treated with 40 g/mL of the conjugates and left overnight. The next day, a volume of 150 μL of the cell supernatant was transferred to a new 96-well plate and 130 μL of deionized water was added on top of it. Lastly, a volume of 20 μL of Griess reagent was added to the cell supernatant and incubated for 30 min in the dark at room temperature. Griess reagent was prepared according to instructions: 1:1 ratio of N-1-naphthyl-ethylenediamine (0.1%) and phosphoric acid (5%) in sulphanilamide (1%) in distilled water. After incubation, the absorbance of azo chromophore formed color was measured spectrophotometrically at 548 nm using a Nano SPECTROstar microplate reader (BMG LABTECH, Germany). The concentration of nitrite was determined using a NaNO₂ standard curve.

### Measurement of Cell Viability Using MTT Assay

To ensure that the inflammatory response is not due to cell toxicity, cell viability measurements were done using MTT colorimetric assay. On the same cell cultured 96-well plate from the previous experiment, 100 μL of 1 mg/mL MTT solution was added to the adherent RAW264.7 macrophages cells at 37° C. and incubated for 2 h. After that, 100 μL of DMSO was added to the wells to dissolve insoluble formazan crystals. Absorbance was measured spectrophotometrically at 540 nm using a NanoSPECTROstar microplate reader (BMG LABTECH, Germany). Cell viability was then calculated as a percentage of untreated control.

Evaluation of Inflammatory mRNA Expression Levels

Inflammatory mRNA expression levels were evaluated using RT-qPCR (Applied Biosystems, CA, USA). Total RNA was extracted by lysing RAW264.7 cells using Qiazol Lysis Reagent. The extracted RNA was used to synthesize cDNA using RevertAid First Strand cDNA synthesis kit according to manufacturer's protocol. The synthesized cDNA was added with forward and reverse primers, nuclease free water, and maxima SYBR green mix (Thermo Fisher Scientific, MA, USA). The inflammatory cytokines for which mRNA expression levels were measured are: IL-6, TNF-α, and iNOS. All expression was normalized to GAPDH (endogenous control) and the relative fold of mRNA expression levels was calculated using the comparative 2^{-ΔΔCT} method. The PCR reaction includes an initiation stage at 95° C. for 10 min, 40 PCR cycles of denaturation stage for 15 s at 95° C., and annealing and extension for 1 min at 60° C. NCBI Primer-Blast tool (https://www.ncbi.nlm.nih.gov/tools/primer-blast/) was used for primer generation (Thermo Fisher scientific, MA, USA). Table 6 lists the primer sequences of inflammation-related genes.

### Docking Studies

The conjugates were energy minimized using the MacroModel (v9.9) molecular mechanics program and prepared for docking using the LigPrep module in Schrödinger modeling package. Protein crystal structures were prepared using protein preparation wizard. Hydrogens were added, bond orders were assigned, and the missing side chains and loops were added using the Prime package in Schrödinger. The hydrogen bonding network was adjusted by reorienting the hydroxyl and thiol groups and amide groups of Asn, Gln, and His side chains. Neutral and protonated states of His, Asp, and Glu and tautomeric states of His were sampled at pH 7.0 using PROPKA. Following H-bond adjustment, the protein was minimized using the OPLS-2005 force field until the RMSD of heavy atoms converged to 0.30 Å. The receptor grid was constructed, and the docking site was set around the crystalized ligand without constraints. Ligands with a length up to 20 Å were allowed to dock. Glide XP was used for pose generation, and the docking was terminated if two consecutive solutions were within an RMSD of 0.5 Å. The docked poses were again minimized using Prime, VSGB solvation model, and the energies were calculated using OPLS3e force field. Residues within 5 Å of the ligand were treated as flexible.

### 2D-QSAR Studies

Geometry of the tested conjugates was adjusted by the molecular mechanics force field (MM⁺), followed by semi-empirical AM1 method implemented in the HyperChem 8.0 package (http://www.hyper.com). The structures were fully adjusted without constraining any parameters, bringing all geometric variables to their equilibrium values. The energy minimization protocol used the Polak-Ribiere conjugated gradient algorithm. Convergence to a local minimum was achieved when the energy gradient was ≤0.01 kcal mol⁻¹. RHF (Restricted Hartree-Fock) method was used in the spin pairing for the semi-empirical tool.

2D-QSAR studies were performed using the comprehensive descriptors for structural and statistical analysis (CODESSA-Pro) software. The structures of the tested conjugates were uploaded to CODESSA-Pro that included MOPAC capability for the final geometry adjustment. CODESSA-Pro calculated molecular descriptors (constitutional, topological, geometrical, charge-related, semi-empirical, thermodynamical, molecular-type, atomic-type and bond-type descriptors) for the exported conjugates. Different mathematical transformations [including property, 1/property, log(property) and 1/log(property)] of the experimentally observed property/activity of the training set compounds were utilized searching for the best QSAR model. The best multi-linear regression (BMLR) technique was utilized, which is a stepwise search for the best n parameter regression equations (where n stands for the number of descriptors used), based on the highest R² (squared correlation coefficient), R²cvOO (squared cross-validation “leave-one-out, LOO” coefficient), R²cvMO (squared cross-validation “leave-many-out, LMO” coefficient), F (Fisher statistical significance criteria) values, and s (standard deviation). The QSAR models describing the bioactivity of the agents were generated (obeying the thumb rule).

### Results and Discussion

### Chemistry

The synthetic pathway used to produce exemplary ibuprofen (“Ibu”) conjugates 5a-5g containing triazolyl moiety is depicted in Scheme 1. The alkyne component accessible for the click chemistry was developed by treating Ibu with propargyl bromide in the presence of cesium carbonate (Cs₂CO₃) in THF (at 0° C. to room temp., overnight). Further, the alkyne was treated with aromatic azides 4 adopting a modified click chemical technique in the presence of CuSO₄·5H₂O and sodium D-isoascorbate in n-butanol-water under microwave irradiation for 2 h at 70° C. to obtain the desired conjugates 5a-5g with yields of 79%, 86%, 85%, 92%, 87%, 85%, and 89%, respectively.

A similar protocol was used to synthesize the indomethacin conjugates 8a-8g using click chemistry (Scheme 2). However, the reaction yields are lower than the ibuprofen conjugates. The reactions were performed at different temperatures and reaction times. Microwave reaction gave a cleaner reaction with better yields than conventional heating. The conjugates 8a-8g were obtained with yields of 65%, 85%, 85%, 72%, 63%, 67%, and 85%, respectively.

### Anti-Inflammatory Properties

The carrageenan-induced rat paw-edema technique was adopted to determine the anti-inflammatory property of the synthesized conjugates. As shown in Table 1 and FIG. 1, most of the synthesized conjugates showed anti-inflammatory properties with improved potency compared with their parent compound (i.e., ibuprofen or indomethacin). No anti-inflammatory efficacy was observed for compounds 7, 8b, and 8f.

SAR (structure-activity relationship) was studied through the observed anti-inflammatory data (Table 1, FIG. 1). Generally, conjugation of triazolyl heterocycle with ibuprofen scaffold affords enhanced anti-inflammatory properties than that of indomethacin (compound 5c is an exception). Some conjugates with enhanced anti-inflammatory properties were observed relative to their parent drug (compounds 5a, 5b, and 5e show % potency of 117.6, 116.5, 109.1, respectively, while ibuprofen (parent compound) showed % potency of 97.2).

For the ibuprofen-triazole conjugates, the chloro-substituted phenyl triazoles showed higher anti-inflammatory properties than those having methyl/methoxy substituted phenyls, as shown in compounds 5a/5b/5d/5e/5f. Additionally, the ortho-substituted phenyl conjugates showed higher anti-inflammatory properties than the para-substituted conjugates as shown in pairs 5a/5b and 5e/5f. The last correlation was observed for indomethacin-triazole conjugates as shown in pairs 8a/8b and 8e/8f.

Conjugates 5a and 5e that showed high acute anti-inflammatory properties also demonstrated considerable potency after 24 h relative to their parent compound (% inhibition of edema after 24 h was 22.5, 42.1, and 11.2 for 5a, 5e, and ibuprofen, respectively). Conjugate 8a showed mild acute anti-inflammatory activity, with enhanced property after 24 h compared with its parent compound (% inhibition of edema after 24 h=33.3, 12.0 for 8a and indomethacin, respectively).

TABLE 1
Anti-inflammatory properties of the exemplary conjugates and parent compounds.

| | Mean edema thickness “mm” (% inhibition of edema ± standard error) | | | | | |
|---|---|---|---|---|---|---|
| Compd. | 1 h | 2 h | 3 h | 4 h | 24 h | % Potency^{a} |
| Control | 0.863 ± 0.02 | 0.713 ± 0.03 | 0.723 ± 0.02 | 0.713 ± 0.01 | 0.920 ± 0.07 | — |
| | (0.00 ± 0.01) | (0.00 ± 0.02) | (0.00 ± 0.01) | (0.00 ± 0.03) | (0.00 ± 0.04) | |
| Indo | 0.510 ± 0.03** | 0.353 ± 0.05** | 0.137 ± 0.01** | 0.350 ± 0.04** | 0.810 ± 0.03** | 100 |
| | (40.9 ± 1.2) | (50.5 ± 2.1) | (81.1 ± 1.8) | (50.9 ± 1.1) | (12.0 ± 2.1) | |
| Ibu | 0.480 ± 0.04** | 0.403 ± 0.09** | 0.153 ± 0.01** | 0.563 ± 0.02* | 0.817 ± 0.07* | 97.2 |
| | (44.4 ± 2.5) | (43.5 ± 2.9) | (78.8 ± 0.9) | (21.0 ± 1.1) | (11.2 ± 0.6) | |
| 3 | 0.727 ± 0.05* | 0.589 ± 0.01* | 0.103 ± 0.04** | 0.473 ± 0.03* | 0.920 ± 0.02* | 105.8 |
| | (15.8 ± 1.9) | (17.4 ± 0.9) | (85.8 ± 1.1) | (33.7 ± 1.2) | (0.00 ± 0.3) | |
| 5a | 0.607 ± 0.04* | 0.490 ± 0.03* | 0.033 ± 0.01** | 0.387 ± 0.06** | 0.713 ± 0.02** | 117.6 |
| | (29.7 ± 1.1) | (31.3 ± 0.8) | (95.4 ± 0.7) | (45.7 ± 1.4) | (22.5 ± 0.9) | |
| 5b | 0.360 ± 0.01** | 0.217 ± 0.07** | 0.040 ± 0.01** | 0.237 ± 0.08** | 0.843 ± 0.09** | 116.5 |
| | (58.3 ± 0.7) | (69.6 ± 2.1) | (94.5 ± 0.4) | (66.8 ± 1.6) | (8.4 ± 1.2) | |
| 5c | 0.863 ± 0.08* | 0.540 ± 0.01* | 0.527 ± 0.03* | 0.530 ± 0.05* | 0.920 ± 0.01* | 33.4 |
| | (0.00 ± 1.0) | (24.3 ± 1.4) | (27.1 ± 0.7) | (25.7 ± 0.9) | (0.00 ± 0.2) | |
| 5d | 0.257 ± 0.08** | 0.206 ± 0.04** | 0.173 ± 0.01** | 0.490 ± 0.07** | 0.920 ± 0.09* | 93.8 |
| | (70.2 ± 2.2) | (71.1 ± 1.5) | (76.1 ± 0.8) | (31.3 ± 1.1) | (0.00 ± 1.7) | |
| 5e | 0.197 ± 0.02** | 0.161 ± 0.08** | 0.083 ± 0.02** | 0.660 ± 0.09* | 0.533 ± 0.01* | 109.1 |
| | (77.2 ± 1.9) | (77.4 ± 2.2) | (88.5 ± 2.0) | (7.4 ± 1.8) | (42.1 ± 0.4) | |
| 5f | 0.703 ± 0.09* | 0.270 ± 0.05* | 0.300 ± 0.07* | 0.700 ± 0.10* | 0.897 ± 0.08* | 72.1 |
| | (18.5 ± 2.5) | (62.1 ± 2.2) | (58.5 ± 2.4) | (1.8 ± 0.7) | (2.5 ± 0.9) | |
| 5g | 0.570 ± 0.05** | 0.230 ± 0.02** | 0.249 ± 0.07** | 0.373 ± 0.04** | 0.920 ± 0.02* | 80.9 |
| | (34.0 ± 1.8) | (67.7 ± 1.1) | (65.6 ± 2.9) | (47.7 ± 1.5) | (0.00 ± 0.5) | |
| 8a | 0.863 ± 0.07* | 0.393 ± 0.08* | 0.488 ± 0.07* | 0.521 ± 0.03* | 0.614 ± 0.09* | 40.1 |
| | (0.00 ± 1.5) | (44.9 ± 1.7) | (32.5 ± 1.5) | (26.9 ± 1.1) | (33.3 ± 2.4) | |
| 8c | 0.750 ± 0.08* | 0.466 ± 0.02* | 0.403 ± 0.01** | 0.340 ± 0.04** | 0.920 ± 0.04* | 54.6 |
| | (13.1 ± 1.7) | (34.6 ± 0.9) | (44.3 ± 0.6) | (52.3 ± 0.8) | (0.00 ± 1.0) | |
| 8d | 0.863 ± 0.07* | 0.618 ± 0.09* | 0.723 ± 0.04* | 0.713 ± 0.03* | 0.920 ± 0.02* | — |
| | (0.00 ± 0.8) | (13.3 ± 1.9) | (0.00 ± 0.5) | (0.00 ± 0.6) | (0.00 ± 0.4) | |
| 8e | 0.863 ± 0.01* | 0.667 ± 0.05* | 0.536 ± 0.07* | 0.683 ± 0.08* | 0.920 ± 0.01* | 31.8 |
| | (0.00 ± 0.2) | (6.5 ± 1.0) | (25.9 ± 1.3) | (4.2 ± 1.9) | (0.00 ± 0.3) | |
| 8g | 0.690 ± 0.02** | 0.394 ± 0.08** | 0.456 ± 0.01** | 0.462 ± 0.04** | 0.920 ± 0.05* | 45.5 |
| | (20.0 ± 0.7) | (44.7 ± 1.7) | (36.9 ± 0.5) | (35.2 ± 0.9) | (0.00 ± 1.2) | |

^{a}Potency is the % inhibition of the edema thickness of the synthesized agent relative to indomethacin at 3 h.
Statistical analysis was conducted by one-way ANOVA (*p < 0.05, **p < 0.001).

### Peripheral Analgesic Property

In-vivo acetic acid-induced abdominal writhing assay in mice was performed for peripheral analgesic testing of the conjugates (10 mg/kg “animal body weight” indomethacin mol equivalent). Table 2 summarizes the observed results. All of the synthesized ibuprofen-triazole conjugates showed peripheral analgesic properties with higher potencies (% potency=88.2-121.9) than their parent compound (% potency of ibuprofen=81.5). Additionally, few of the synthesized indomethacin-triazole conjugates showed enhanced biological properties (% potency of 8e and 8g: 112.5 and 134.1, respectively) compared with their parent compound (% potency of indomethacin: 100).

SAR was studied based on the observed biological properties. Ibuprofen-triazole conjugates-containing halogenated phenyl are more effective than those with methyl- or methoxyphenyl compounds as shown in compounds 5b/5c/5d/5f and 5a/5e. The conjugate with chlorophenyl substituent is more potent than that of fluorophenyl ring as shown in pairs 5b/5c (% potency: 121.9 and 116.0, respectively). Additionally, the para-substituted phenyl conjugates showed higher analgesic properties than those of ortho-substituted phenyl conjugates as shown in pairs 5a/5b (% potency: 116.0 and 121.9, respectively) and 5e/5f (% potency: 88.2 and 111.4, respectively).

In contrast, the ortho-substituted phenyl indomethacin-triazole conjugates showed higher peripheral analgesic properties than the para-substituted phenyl conjugates as shown in pairs 8a/8b and 8e/8f (% potency: 68.7/64.5 and 112.5/93.0, respectively). Additionally, the methyl- and methoxyphenyl containing indomethacin-triazole conjugates showed higher potency than the halogenated phenyl containing-conjugates as shown in conjugates 8d/8f/8c/8b and 8e/8a.

TABLE 2
Peripheral analgesic properties of the exemplary
conjugates and parent compounds.

| Compd. | Writing reflex ± SE | % Inhibition/protection | Potency^{a} |
|---|---|---|---|
| Control | 40 ± 3.2 | 0 | — |
| Indo | 11.3 ± 1.6** | 71.8 | 100.0 |
| Ibu | 16.6 ± 0.7** | 58.5 | 81.5 |
| 3 | 11.3 ± 0.9** | 71.8 | 100.0 |
| 5a | 6.7 ± 0.7** | 83.3 | 116.0 |
| 5b | 5.0 ± 0.9** | 87.5 | 121.9 |
| 5c | 6.7 ± 0.5** | 83.3 | 116.0 |
| 5d | 7.0 ± 1.3** | 82.5 | 114.9 |
| 5e | 14.7 ± 1.6** | 63.3 | 88.2 |
| 5f | 8.0 ± 1.4** | 80.0 | 111.4 |
| 5g | 14.0 ± 1.9** | 65.0 | 90.5 |
| 7 | 4.3 ± 0.5** | 89.3 | 124.4 |
| 8a | 20.3 ± 1.8* | 49.3 | 68.7 |
| 8b | 21.5 ± 1.5* | 46.3 | 64.5 |
| 8c | 23.5 ± 1.4* | 41.3 | 57.5 |
| 8d | 15.0 ± 1.6* | 62.5 | 87.0 |
| 8e | 7.7 ± 1.3** | 80.8 | 112.5 |
| 8f | 13.3 ± 1.9** | 66.8 | 93.0 |
| 8g | 1.5 ± 0.4** | 96.3 | 134.1 |

^{a}Potency is the % inhibition/protection compared to indomethacin.

### Central Analgesic Property

Central analgesic property of the synthesized conjugates was performed using the hot plate assay in mice (10 mg/kg “animal body weight” indomethacin mol equivalent). As shown in Table 3, conjugate 8f is superior among all the synthesized conjugates, showing higher potency than its parent compound (% potency: 117.7 and 100 for 8f and indomethacin, respectively). Conjugate 5a also showed central analgesic potency comparable to its parent compound (% potency: 96.5 and 96.1 for 5a and ibuprofen, respectively). Compound 5c showed high analgesic properties at the first-time interval (1.8 folds % protection compared with its parent compound, ibuprofen at 30 min). This analgesic effect of 5c decreased drastically by time (% protection: 18.0 and 54.3 for 5c and ibuprofen, respectively, at 120 min.). Similar results were observed for conjugate 5f (% protection: 90.1, 0.2; 61.7 and 54.3 for 5f and ibuprofen at 30- and 120-min time intervals, respectively.). Conjugate 8f (the most potent conjugate with respect to central analgesic property) showed almost stable activities through all the experimental time intervals, similar to its parent compound (% protection: 66.3, 92.4, 68.4, 66.5; 87.1, 80.9, 57.9, 56.5 for 8f and indomethacin at 30, 60, 90, and 120 min, respectively).

SAR was studied based on the biological observations. For the ibuprofen-triazole conjugates, the halogenated phenyl containing-conjugates showed higher central analgesic properties relative to the methyl- or methoxyphenyl containing-compounds as shown in compounds 5b/5c/5d/5f and 5a/5e. The ortho-substituted phenyl containing triazoles are more potent than the para-substituted phenyl conjugates as shown in pairs 5a/5b (% potency: 96.5 and 48.0, respectively) and 5e/5f (% potency: 49.6 and 0.4, respectively). In contrast, the methyl-, methoxyphenyl containing indomethacin-triazole conjugates showed higher bio-activities than the fluoro-, chlorophenyl-containing conjugates (8a is an exception), as shown in conjugates 8d/8f compared with 8b/8c (% potency: 86.0, 117.7, 17.2, and 85.1, respectively).

TABLE 3
Central analgesic properties of the exemplary conjugates and parent compounds.

| | Latency period ± SE “standard error”, second (% | | | | |
|---|---|---|---|---|---|
| | Protection) | | | | |
| | After 30 | After 60 | After | After | |
| Compd. | min. | min. | 90 min. | 120 min. | Potency^{a} |
| Control | 6.68 ± 1.0 | 7.27 ± 1.4 | 8.91 ± 0.9 | 9.01 ± 1.1 | — |
| | (0.0) | (0.0) | (0.0) | (0.0) | |
| Indo | 12.50 ± | 13.15 ± | 14.07 ± 1.2** | 14.10 ± 1.9** | 100 |
| | 2.0** | 1.6** (80.9) | (57.9) | (56.5) | |
| | (87.1) | | | | |
| Ibu | 10.80 ± | 11.00 ± | 11.90 ± 1.5** | 13.90 ± 2.2** | 96.1 |
| | 0.8** | 1.3** (51.3) | (33.6) | (54.3) | |
| | (61.7) | | | | |
| 3 | 10.37 ± | 10.78 ± | 10.62 ± 2.2** | 10.35 ± 1.7** | 26.4 |
| | 1.6** | 2.1** (48.3) | (19.2) | (14.9) | |
| | (55.2) | | | | |
| 5a | 11.97 ± | 12.47 ± | 11.98 ± 2.6** | 13.92 ± 2.3** | 96.5 |
| | 2.5** | 2.0** (71.5) | (34.5) | (54.5) | |
| | (79.2) | | | | |
| 5b | 12.30 ± | 10.27 ± | 11.14 ± 1.5** | 11.45 ± 2.0** | 48.0 |
| | 1.6** | 1.9** (41.3) | (25.0) | (27.1) | |
| | (84.1) | | | | |
| 5c | 14.17 ± | 15.0 ± 2.3 | 14.91 ± 2.5** | 10.63 ± 1.8** | 31.9 |
| | 2.6** | **(106.3) | (67.3) | (18.0) | |
| | (112.1) | | | | |
| 5d | 11.02 ± | 9.86 ± 0.7* | 10.69 ± 0.9* | 9.12 ± 1.1* | 2.1 |
| | 1.4** | (35.6) | (20.0) | (1.2) | |
| | (65.0) | | | | |
| 5e | 12.61 ± | 10.53 ± 1.8* | 12.12 ± 1.7* | 11.53 ± 1.3* | 49.6 |
| | 2.1** | (44.8) | (36.03) | (28.0) | |
| | (88.8) | | | | |
| 5f | 12.7 ± | 11.40 ± 1.5* | 10.60 ± 1.9* | 9.03 ± 1.6* | 0.4 |
| | 0.9* | (56.8) | (19.0) | (0.2) | |
| | (90.1) | | | | |
| 5g | 12.5 ± | 12.59 ± | 10.60 ± 2.6** | 11.54 ± 2.2** | 49.7 |
| | 2.1** | 1.5** (73.2) | (19.0) | (28.1) | |
| | (87.1) | | | | |
| 7 | 6.68 ± | 9.54 ± 1.6* | 11.18 ± 1.5* | 10.78 ± 1.8* | 34.7 |
| | 2.0* (0.0) | (31.2) | (25.5) | (19.6) | |
| 8a | 12.19 ± | 13.51 ± | 13.85 ± 1.4** | 13.63 ± 1.3** | 90.8 |
| | 1.9** | 1.7** (85.8) | (55.4) | (51.3) | |
| | (82.5) | | | | |
| 8b | 6.84 ± | 8.31 ± 1.9* | 9.91 ± 1.7* | 9.88 ± 2.0* | 17.2 |
| | 1.4* (2.4) | (14.3) | (11.2) | (9.7) | |
| 8c | 11.32 ± | 9.39 ± 1.1* | 11.40 ± 1.6* | 13.34 ± 1.8* | 85.1 |
| | 0.8* | (29.2) | (27.9) | (48.1) | |
| | (69.5) | | | | |
| 8d | 13.21 ± | 12.65 ± | 13.37 ± 1.6** | 13.39 ± 1.2** | 86.0 |
| | 2.5** | 2.0** (74.0) | (50.1) | (48.6) | |
| | (97.8) | | | | |
| 8e | 11.30 ± | 13.23 ± | 11.36 ± 2.5** | 11.61 ± 2.2** | 51.2 |
| | 1.1** | 1.6** (82.0) | (27.5) | (28.9) | |
| | (69.2) | | | | |
| 8f | 11.11 ± | 13.99 ± | 15.00 ± 2.5** | 15.00 ± 2.6** | 117.7 |
| | 1.6** | 1.9** (92.4) | (68.4) | (66.5) | |
| | (66.3) | | | | |
| 8g | 10.46 ± | 10.57 ± | 12.58 ± 2.0** | 9.25 ± 2.3* | 4.8 |
| | 2.4** | 2.7** (45.4) | (41.2) | (2.7) | |
| | (56.6) | | | | |

^{a}Potency is that at 120 min. compared to indomethacin.

### Ulcerogenic Liability

The synthesized anti-inflammatory conjugates (3, 5a, 5b, 5d, 5e) were tested for ulcerogenic liability in mice. As shown in Table 4, none of the synthesized potential conjugates (5a, 5b, 5d, 5e) showed ulcers or erosions to the gastric of tested animals, which demonstrated their safe applicability for oral administration.

### Toxicological Bioassay

The synthesized anti-inflammatory conjugates (3, 5a, 5b, 5d, 5e) were tested for their toxicological effect in mice. Five times of the anti-inflammatory dose was orally administrated. No toxic symptoms or mortality was observed using any of the tested conjugates.

TABLE 4
Ulcerogenic liability of the tested compounds.

| | | Number of | % Incidence | Average | Average | |
|---|---|---|---|---|---|---|
| | | animals | of ulcer | of ulcer | severity | Ulcer |
| Entry | Compd. | with ulcer | divided by 10 | number | of ulcer | index |
| 1 | Control | 0/6 | 0 | 0 | 0 | 0 |
| 2 | Indo | 6/6 | 10 | 2 | 1.67 | 13.67 |
| 3 | Ibu | 2/6 | 3.33 | 0.33 | 0.67 | 4.33 |
| 4 | 3 | 2/6 | 3.33 | 0.33 | 0.67 | 4.33 |
| 5 | 5a | 0 | 0 | 0 | 0 | 0 |
| 6 | 5b | 0 | 0 | 0 | 0 | 0 |
| 7 | 5d | 0 | 0 | 0 | 0 | 0 |
| 8 | 5e | 0 | 0 | 0 | 0 | 0 |

### Inhibition of COX-1 and COX-2

The inhibitory properties of the synthesized anti-inflammatory conjugates (3, 5a, 5b, 5d, and 5e) against COX-1 and COX-2 were studied by a standard technique according to the manufacturer's instructions. As shown in Table 5, all of the synthesized conjugates showed enhanced selectivity index [SI=IC_{50 (COX-1)}/IC_{50 (COX-2)}] towards COX-2 compared with COX-1. Conjugate 5a is superior with high SI of 23.096. Conjugate 5b also showed competent SI value (SI=9.619). The SI values follow a similar trend to the anti-inflammatory % potency for most of the tested conjugates (SI: 2.262, 23.096, 9.619 and 2.158; % potency: 105.8, 117.6, 116.5 and 93.8 for conjugates 3, 5a, 5b and 5d, respectively). Further, conjugate 5e showed higher potency against COX-1 relative to its parent compound (i.e., ibuprofen), which is consistent the anti-inflammatory properties observed (IC₅₀ against COX-1: 5.417 and 13.16 μM; % potency: 109.1 and 97.2, for 5e and Ibu, respectively). Additionally, conjugate 5e showed a mild SI value (COX-1/COX-2), similar to its parent compound (SI: 0.387 and 0.106 for 5e and Ibu, respectively).

TABLE 5
COX-1 and COX-2 inhibitory properties of the tested compounds.

| | IC₅₀, μM ± SD | | |
|---|---|---|---|
| Compd. | COX-1 | COX-2 | SI |
| Indo | 0.354 ± 0.01** | 3.239 ± 0.09** | 0.109 |
| IBU | 13.16 ± 0.37** | 124.2 ± 3.53* | 0.106 |
| 3 | 19.45 ± 0.55** | 8.599 ± 0.24** | 2.262 |
| 5a | 17.16 ± 0.41** | 0.743 ± 0.02** | 23.096 |
| 5b | 118.8 ± 3.4** | 12.35 ± 0.35** | 9.619 |
| 5d | 9.638 ± 0.27** | 4.467 ± 0.13** | 2.158 |
| 5e | 5.417 ± 0.15** | 13.99 ± 0.40** | 0.387 |

Evaluation of NO production of LPS-Stimulated RAW264.7

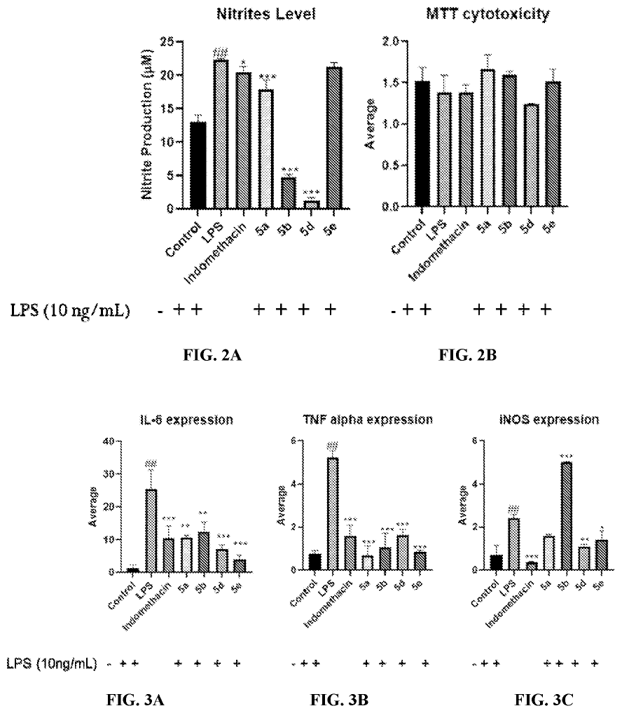

The anti-inflammatory responses of ibuprofen conjugates (5a, 5b, 5d, 5e) and indomethacin (reference standard) were assessed on LPS-stimulated RAW264.7 cells. The nitrite production in the supernatant in the culture medium was measured using Greiss reaction. Based on the nitrite standard curve, it was found that the baseline of NO production in RAW 264.7 cells is 12.8 M. When RAW 264.7 cells were stimulated with LPS, NO production was significantly increased to 22 μM compared with the control (P***<0.001). As shown in FIG. 2A, indomethacin (positive control) decreased LPS-stimulated NO production to 18.8±2.2 μM (P*<0.05). The ibuprofen conjugates, 5a, 5b, and 5d, significantly reduced the LPS-stimulated NO production to 17.7 μM±3.5, 4.88±1 μM and 1.45±1.3 μM, respectively, compared with LPS-stimulated cells (P***<0.001, P***<0.001, P***<0.001, respectively). These results demonstrate that these ibuprofen conjugates 5a, 5b and 5d are superior to the positive control (indomethacin) in reducing NO production in LPS-stimulated RAW264.7 cells and highlighting the potential anti-inflammatory effect. The ibuprofen conjugate 5e showed an insignificant decrease (P>0.05) in NO production in comparison to LPS-stimulated cells.

Cytotoxicity of the macrophages was evaluated using MTT assay. As shown in FIG. 2B, no significant cytotoxicity was observed for LPS-stimulated cells (without treatment by the conjugates or indomethacin) and LPS-stimulated cells treated with ibuprofen conjugates and indomethacin (positive control), in comparison to control.

Evaluation of mRNA Levels of Inflammatory Cytokines in LPS-Stimulated RAW264.7 Cells

The effect of these conjugates on the pro-inflammatory mRNA markers (IL-6, TNF-α and iNOS) was determined. The mRNA levels of IL-6, TNF-α and iNOS were measured using real-time qPCR (RT-qPCR). The mRNA sequences used for the primers in RT-qPCR are provided in Table 6. LPS resulted in a significant increase in the mRNA levels of IL-6, TNF-α and iNOS (P***<0.001, P***<0.001, P**<0.01, respectively, in comparison to control). Treatment with the ibuprofen conjugates significantly decreased mRNA levels of IL-6, TNF-α and iNOS in LPS-stimulated RAW264.7 cells (FIGS. 3A-3C).

As shown in FIG. 3A, a reduction in the IL-6 gene expression levels was detected in LPS-stimulated RAW264.7 cells treated with the ibuprofen conjugates: 61.3, 42, 66.2 and 82% reduction for 5a, 5b, 5d and 5e respectively (P**<0.01, P**<0.01, P***<0.001, P***<0.001, respectively, in comparison to LPS-stimulated cells only). The positive control, indomethacin, showed a 50% reduction in mRNA levels of IL-6.

As shown in FIG. 3B, a reduction in the TNF-α gene expression levels was detected in LPS-stimulated RAW264.7 cells treated with the ibuprofen conjugates: 88, 82, 72, 86% reduction for 5a, 5b, 5d and 5e respectively (P***<0.001, P***<0.001, P***<0.001, P***<0.001, respectively, in comparison to LPS-stimulated cells only). The positive control, indomethacin, showed a 74% reduction in mRNA levels of TNF-α.

As shown in FIG. 3C, a reduction in iNOS gene expression levels was detected in LPS-stimulated RAW264.7 cells treated with the ibuprofen conjugates except for 5b, 34, 59 and 42% reduction of mRNA levels were observed for 5a, 5d and 5e respectively (P>0.05. P**<0.01, P*<0.05, respectively, in comparison to LPS-stimulated cells only). The positive control, indomethacin, showed an 85% reduction in mRNA levels of iNOS.

TABLE 6
mRNA sequences used for RT-qPCR.

| Target | | | |
|---|---|---|---|
| mRNA | Primer sequence (5′-3′) | | Tm |
| IL-6 | Forward: | GATGCTACCAAACTGGATATAA | 55 |
| | | TCAG (SEQ ID NO: 1) | |
| | Reverse: | CTCTGAAGGACTCTGGCTTTG | 58 |
| | | (SEQ ID NO: 2) | |
| TNF-α | Forward: | GAACTCCAGGCGGTGCCTAT | 63 |
| | | (SEQ ID NO: 3) | |
| | Reverse: | TGAGAGGGAGGCCATTTGGG | 63 |
| | | (SEQ ID NO: 4) | |
| iNOS | Forward: | GGAACCTACCAGCTCACTCTGG | 63 |
| | | (SEQ ID NO: 5) | |
| | Reverse: | TGCTGAAACATTTCCTGTGCTGT | 60 |
| | | (SEQ ID NO: 6) | |
| GAPDH | Forward: | CTTTGTCAAGCTCATTTCCTGG | 57 |
| | | (SEQ ID NO: 7) | |
| | Reverse: | TCTTGCTCAGTGTCCTTGC | 58 |
| | | (SEQ ID NO: 8) | |

### Molecular Modeling Studies

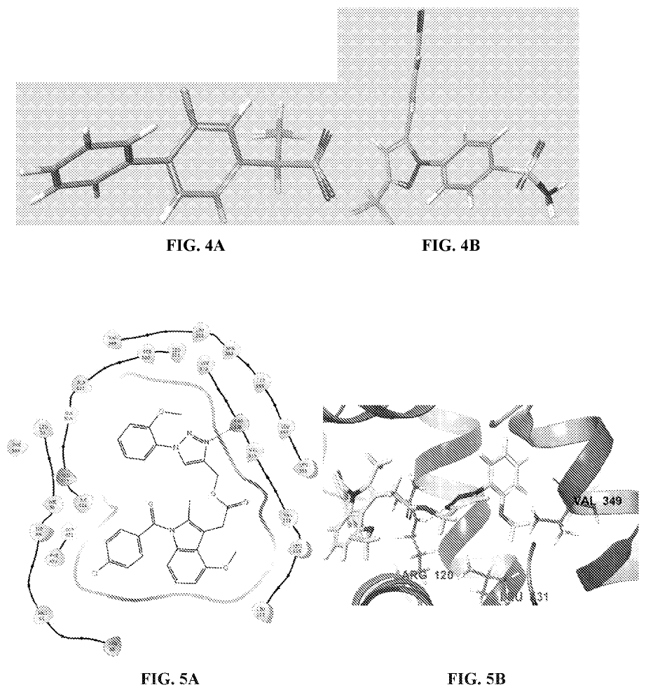

Among the compounds tested, conjugate 5a is highly selective for COX-2, with a SI of 23.096 (Table 5). Conjugate 5e is more potent in inhibiting COX-1 and is about three-fold less active in inhibiting COX-2. To investigate the underlying molecular interactions leading to the selectivity of the conjugates, docking studies were performed using the Glide program of the Schrodinger software, v2020-1. Glide extra precision (XP) mode, which uses a more sophisticated scoring system, was used for docking. COX-2 crystal structure co-crystalized with selective inhibitor SC-558. PDB accession number 6COX, and COX-1 structure crystalized with COX-1 selective drug flurbiprofen (PDB entry: 3N8W) were used for docking simulations. Glide poses were first validated by docking the native ligands of the 6COX and 3N8W structures in the receptor active site. Superimposition of the docked poses with their respective bioactive ligand conformations (FIGS. 4A and 4B) provided a low root mean square deviation (RM4SD) of 0.088 Å and 0.080 Å, respectively. It shows that Glide XP docking could reproduce the ligand conformation in the COX crystal structures accurately.

The binding mode of conjugate 5e in the COX-1 active site (PDB 3N8W) showed that the triazole ring lies perpendicular to the plane of the 2-OCH₃ phenyl ring (FIGS. 5A and 5B). In this orientation, the triazole ring makes a H-bonding interaction with Arg120, which is located at the entrance of the cyclooxygenase active site. The carboxylic acid of arachidonic acid (AA), the cyclooxygenase substrate, makes a salt bridge interaction with Arg120 of COX-1. The 2′-methoxy substituent on the phenyl ring of conjugate 5e fits into a hydrophobic groove near the enzyme active site. This 2-methoxy substituent may impart COX-1 inhibitory activity by interacting with residues Leu531 and Val349. Val349 in COX-1 helps position the substrate AA in the active site and confers catalytic activity to the enzyme leading to maximum PGG2 production. In indomethacin, the 2-methyl group on the indole ring interacts with Val349.

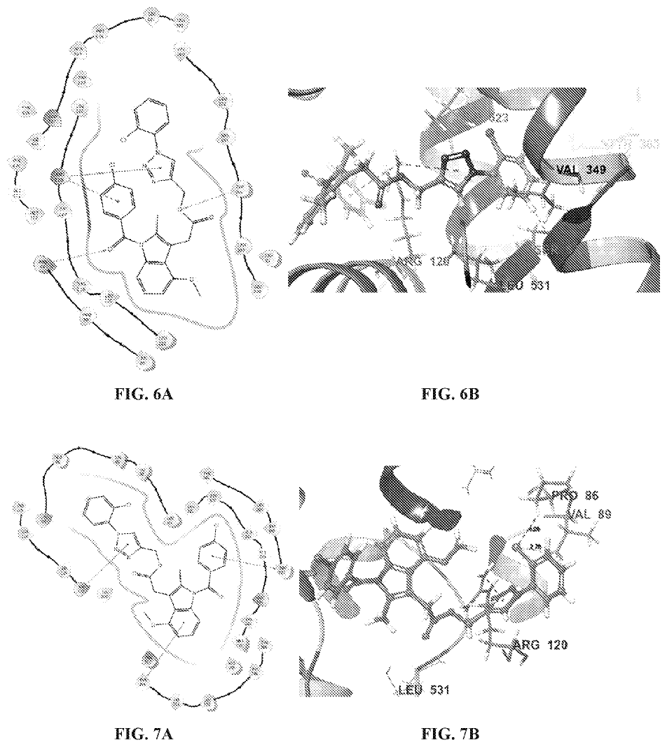

The binding mode of conjugate 5a (FIGS. 6A and 6B) was studied for its weak COX-1 inhibitory activity. The triazole ring got flipped and is substantially coplanar with the 2-chloro phenyl ring. The chloro substituent makes vdW contact with ILe523. The drop of COX-1 potency in conjugate 5a may be attributed to the loss of interactions with Leu531 or Val 349. H-bonding interactions with Arg120 were also absent. A cation-pi interaction between Arg120 and the triazole ring may occur.

The experimental data for conjugates 5a and 5e agree with the observed Glide XP scores of −5.9 kcal/mol and −5.5 kcal/mol, respectively. Since docking score is not a reliable indicator of binding free energies, ΔGMM-GBSA was calculated on the docked poses of 5a and 5e. A more significant difference in the binding free energy estimations (−87 kcal/mol for 5e and −68 kcal/mol for 5a) was observed, which correlate with the observed COX-1 IC₅₀s. The XP docking scores of 5b and 5d in structure 3N8W did not correlate with observed experimental data; however, their MM-GBSA ΔG binding scores of −81.98 kcal/mol and −75.91 kcal/mol aligned well with observed potency. Overall, the COX-1 MM-GBSA ΔG binding scores of conjugates 5a, 5b, 5d, and 5e correlated with observed potency values in Table 5.

For COX-2, the substrate AA does not make a salt bridge with Arg120 but makes an H-bonding interaction with Tyr385 and Ser530 (6). COX-2 has a larger side pocket formed by substituting His513 (in COX-1) with Arg 513 and mutation of Ile434 and 523 (in COX-1) to smaller valine residues. In the COX-2 structure, the Leu531 is more flexible, oriented differently, and may not involve in substrate binding or COX activity. In contrast, Leu531 mutations in COX-1 may lead to more than 90% loss of maximal cyclooxygenase activity. To study the greater COX-2 potency of 5a and a relative loss of activity of 5e, these two compounds in the COX-2 crystal structure (PDB ID: 6COX) were docketed.

The Glide XP could only dock 5a (FIGS. 7A and 7B), but did not give any pose for conjugate 5e. The docking simulations were performed using default conditions, including Coulomb-van der Waals energy cut-off of 0 kcal/mol for pose filtering. To accept higher energy poses of 5e, the threshold was relaxed to incorporate poses with the combined Coulomb and van der Waals interaction energy of 2 kcal/mol. However, the Glide XP could not retrieve any pose for conjugate 5e, showing that 5e may not optimally fit into the COX-2 active site. Switching the Glide XP mode to a less accurate Glide standard precision (SP) mode did dock 5e; however, the SP pose was not considered for analysis. ΔGMM-GBSA calculations on the XP pose of compound 5a gave binding free energy of −69 kcal/mol, which was marginally better than the binding free energy of 5a (68 kcal/mol) in the COX-1 active site. The triazole ring of 5a in the COX-2 structure is oriented out of the plane and interacts with Arg120 via cation-pi interactions (FIGS. 7A and 7B). The presence of the 2-chloro substituent resulted in COX-2 selectivity, probably by interacting with Pro86 and Val89 through hydrophobic and Van der Waals interactions. Val89 is a residue of the membrane binding domain of COX and is shown to confer greater COX-2 inhibitory potency. Conjugates 5b and 5d were then docketed in the 6COX active site.

In agreement with the SAR, improved docking (XP) and MMGBSA scores (−2.681, −59.66 kcal/mol) were observed for 5d over 5b (1.145, −39.94 kcal/mol). MMGBSA binding free energy of 5a, 5b, and 5d also aligned with the COX-2 experimental data in Table 5.

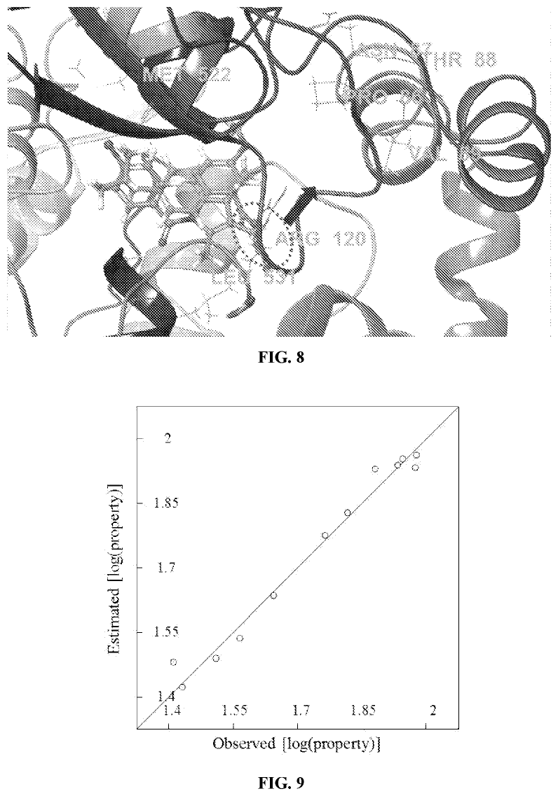

Compared to Indomethacin and Ibuprofen, conjugate 5e is more potent and selective for COX-2. As shown in FIG. 8, an overlay of the docked poses of Indomethacin and Ibuprofen were observed in the COX2 crystal structure, 6COX.

The carboxylic acid group interacted with Arg120, without contacts with Pro 86 and Val89. The binding mode shows that the selectivity and potency for COX-2 may be achieved by appending groups that can target the loop residues, including Pro86, Asn87, Thr88, and Val89. The carboxylic acid of indomethacin was used as the seed group to which a phenyl substituted triazole ring was conjugated. The position of the substituent on the phenyl ring played a role in achieving selectivity and potency for COX-2, as shown by the 2-chloro substituent of 5a.

### Anti-Inflammatory QSAR Model

QSAR can utilize the physicochemical parameters (descriptors) to express mathematically the biological properties. It is usable to rationalize the bio-properties exhibited. Prediction of new hits/leads based on a pre-assigned model and identification of parameters for bio-properties optimization are benefits of the QSAR technique.

CODESSA-Pro software was considered for the current QSAR studies. A robust three descriptor QSAR model (R²=0.979, R²cvOO=0.951, R²cvMO=0.963) describes the anti-inflammatory observations of the tested compounds (Tables 7-9 and FIG. 9).

TABLE 7
Descriptors of the QSAR model for the exemplary conjugates.

| ID | Coefficient | s | t | Descriptor |
|---|---|---|---|---|
| 0 | 2.4754 | 0.045 | 54.69 | Intercept |
| D₁ | −0.0148805 | 0.003 | −5.545 | H-donors PSA (version 2) |
| D₂ | −0.0021036 | 0.0001 | −13.16 | WNSA-1 Weighted PNSA |
| | | | | (PNSA1*TMSA/1000) |
| | | | | (MOPAC PC) |
| D₃ | −0.0598335 | 0.004 | −13.426 | RNCS Relative negative |
| | | | | charged SA |
| | | | | (SAMNEG*RNCG) (Zefirov PC) |

N = 12,
n = 3,
R² = 0.979,
R²cvOO = 0.951,
R²cvMO = 0.963,
F = 123.9,
s² = 0.001,
log(% inhibition of edema thickness at 3 h) = 2.4754 − (0.0148805 × D₁) − (0.0021036 × D₂) − (0.0598335 × D₃)

TABLE 8
Observed and estimated anti-inflammatory properties for the
exemplary conjugates according to the BMLR-QSAR model.

| Com- | log(observed | Observed | log(estimated | Estimated | |
|---|---|---|---|---|---|
| pound | property) | property | property) | property | Error^{a} |
| 3 | 1.93349 | 85.8 | 1.93775 | 86.6 | −0.8 |
| 5a | 1.97955 | 95.4 | 1.95999 | 91.2 | 4.2 |
| 5b | 1.97543 | 94.5 | 1.93178 | 85.5 | 9.0 |
| 5c | 1.43297 | 27.1 | 1.41998 | 26.3 | 0.8 |
| 5d | 1.88138 | 76.1 | 1.92863 | 84.8 | −8.7 |
| 5e | 1.94694 | 88.5 | 1.95295 | 89.7 | −1.2 |
| 5f | 1.76716 | 58.5 | 1.77554 | 59.6 | −1.1 |
| 5g | 1.8169 | 65.6 | 1.82804 | 67.3 | −1.7 |
| 8a | 1.51188 | 32.5 | 1.48976 | 30.9 | 1.6 |
| 8c | 1.6464 | 44.3 | 1.63371 | 43.0 | 1.3 |
| 8e | 1.4133 | 25.9 | 1.4783 | 30.1 | −4.2 |
| 8g | 1.56703 | 36.9 | 1.536 | 34.4 | 2.5 |

^{a}Error is the difference between the observed and estimated property.

TABLE 9
Molecular descriptor values of the QSAR
model for the exemplary conjugates.

| | Descriptors^{a} | | |
|---|---|---|---|
| Comopund | D₁ | D₂ | D₃ |
| 3 | 11.92823 | 89.51916 | 2.87195 |
| 5a | 14.79101 | 140.3812 | 0 |
| 5b | 12.88249 | 163.8141 | 0.1223 |
| 5c | 11.92823 | 148.1222 | 9.46504 |
| 5d | 18.60805 | 121.181 | 0.24984 |
| 5e | 1.90852 | 150.1909 | 2.97673 |
| 5f | 12.88249 | 136.0808 | 3.70859 |
| 5g | 9.06546 | 216.654 | 0.94766 |
| 8a | 15.74527 | 271.1091 | 3.02568 |
| 8c | 12.88249 | 248.1973 | 2.13735 |
| 8e | 14.31388 | 298.4915 | 2.61049 |
| 8g | 18.13092 | 289.9739 | 0.99622 |

^{a}D₁ = H-donors PSA (version 2), D₂ = WNSA-1 Weighted PNSA (PNSA1*TMSA/1000) (MOPAC PC), D₃ = RNCS Relative negative charged SA (SAMNEG*RNCG) (Zefirov PC).

The charge-related descriptor H-donors PSA (t: −5.545) is negatively participated in the QSAR model with a coefficient value of −0.0149). Therefore, a conjugate with a high mathematical descriptor value indicates a low estimated biological property, as shown in conjugates 5c and 5e (descriptor value: 11.928 and 1.909; estimated anti-inflammatory property: 26.3 and 89.7 for 5c and 5e, respectively). The partial positively charged surface area can be calculated by equ. (1).

$$\begin{matrix}PPSA 1 = {\sum }_{A} {S}_{A} & A ∈ \{ {δ}_{A} > 0 \}\end{matrix} \qquad ( 1 )$$

where, S_{A} is the positively charged solvent-accessible atomic surface area.

Weighted PNSA is a charge related-descriptor (t: −13.16). Low descriptor value of 5d relative to that of 5c indicates its potent estimated anti-inflammatory property (descriptor value: 121.181 and 148.1222; estimated property: 84.8 and 26.3 for 5d and 5c, respectively), due to its negative coefficient sign (coefficient: −0.002) in the QSAR model. The surface weighted charged partial negative charged surface area WNSA-1 is determined by equ. (2).

$$WNSA 1 = \frac{PNSA 1 \cdot TMSA}{1000} \qquad ( 2 )$$

where, PNSA1 is the partial negatively charged molecular surface area and TMSA is the total molecular surface area.

Relative negative charged surface area is also a charge-related descriptor with a coefficient value of −0.060. This correlates to the enhanced predicted anti-inflammatory property of 5b relative to 5c (descriptor value: 0.1223 and 9.46504; estimated property: 85.5 and 26.3 for 5b and 5c, respectively). The relative negative charge can be determined by equ. (3).

$$\begin{matrix}RNCG = \frac{{δ}_{max}^{-}}{{\sum }_{A} {δ}_{A}} & A ∈ \{ {δ}_{A} > 0 \}\end{matrix} \qquad ( 3 )$$

where,

$${δ}_{max}^{-}$$

is the maximum atomic negative charge in the molecule and 6, is the negative atomic charge in the molecule.

The statistical parameters (F: 123.9, s²: 0.001) and the correlation of the observed and calculated anti-inflammatory properties of the tested conjugates support the results of the QSAR model.

### Peripheral Analgesic QSAR Model

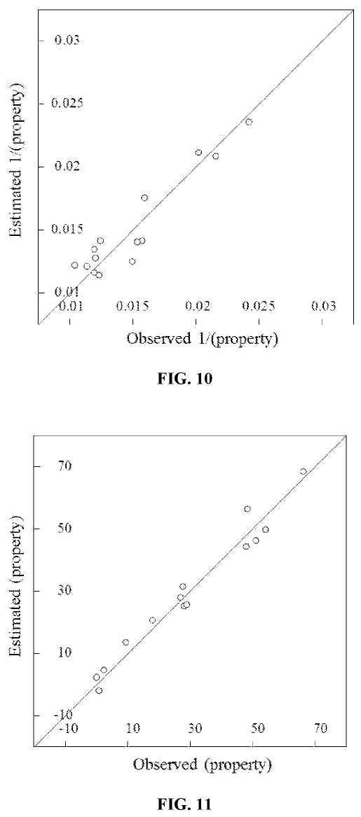

Three descriptor QSAR model expressed the observed peripheral analgesic properties of the tested conjugates (Tables 10-12 and FIG. 10). The semi-empirical descriptor average nucleophilic reactivity index for atom N possess a coefficient value of −0.981123 in the QSAR model determining 1/(property “% inhibition/protection”). The high descriptor value indicates low potent compound, as shown in conjugates 5c and 8c (descriptor values: 0.00721 and 0.00365; estimated % protection/inhibition: 86.0 and 42.5, respectively). Fukui atomic nucleophilic reactivity index can be calculated by equ. (4).

$${N}_{A} = {\sum }_{t ∈ A} {C}_{tHOMO}^{2} / ( 1 - {ε}_{HOMO} ) \qquad ( 4 )$$

where, εHOMO and C_{tHOMO} are the highest occupied molecular energy and coefficients, respectively.

TABLE 10
Descriptors of the QSAR model for the exemplary conjugates.

| ID | Coefficient | s | t | Descriptor |
|---|---|---|---|---|
| 0 | 2.58125 | 0.448 | 5.757 | Intercept |
| D₁ | −0.981123 | 0.203 | −4.827 | Avg. nucleoph. react. index for |
| | | | | atom N |
| D₂ | −0.323865 | 0.057 | −5.659 | Max. atomic state energy for atom H |
| D₃ | −1.28007 | 0.158 | −8.096 | Max. electroph. react. index for |
| | | | | atom C |

N = 14,
n = 3,
R² = 0.895,
R²cvOO = 0.811,
R²cvMO = 0.831,
F = 28.355,
s² = 2.435e−006
1/(% inhibition) = 2.58125 − (0.981123 × D₁) − (0.323865 × D₂) − (1.28007 × D₃)

TABLE 11
Observed and estimated peripheral analgesic properties for
the exemplary conjugates according to the BMLR-QSAR model.

| Com- | 1/(observed | Observed | 1/(estimated | Estimated | |
|---|---|---|---|---|---|
| pound | property) | property | property) | property | Error^{a} |
| 5a | 0.012005 | 83.3 | 0.013395 | 74.7 | 8.6 |
| 5b | 0.011429 | 87.5 | 0.012091 | 82.7 | 4.8 |
| 5c | 0.012005 | 83.3 | 0.011625 | 86.0 | −2.7 |
| 5d | 0.012121 | 82.5 | 0.012785 | 78.2 | 4.3 |
| 5e | 0.015798 | 63.3 | 0.014143 | 70.7 | −7.4 |
| 5f | 0.0125 | 80.0 | 0.014098 | 70.9 | 9.1 |
| 5g | 0.015385 | 65.0 | 0.013976 | 71.6 | −6.6 |
| 8a | 0.020284 | 49.3 | 0.021096 | 47.4 | 1.9 |
| 8b | 0.021598 | 46.3 | 0.020818 | 48.0 | −1.7 |
| 8c | 0.024213 | 41.3 | 0.023524 | 42.5 | −1.2 |
| 8d | 0.016 | 62.5 | 0.01749 | 57.2 | 5.3 |
| 8e | 0.012376 | 80.8 | 0.011431 | 87.5 | −6.7 |
| 8f | 0.01497 | 66.8 | 0.012473 | 80.2 | −13.4 |
| 8g | 0.010384 | 96.3 | 0.012123 | 82.5 | 13.8 |

^{a}Error is the difference between the observed and estimated property.

TABLE 12
Molecular descriptor values of QSAR
model for the exemplary conjugates.

| | Descriptors^{a} | | |
|---|---|---|---|
| Compound | D₁ | D₂ | D₃ |
| 5a | 0.00012 | 7.8146 | 0.0288 |
| 5b | 0.00595 | 7.815 | 0.02525 |
| 5c | 0.00721 | 7.8103 | 0.02583 |
| 5d | 0.00734 | 7.8159 | 0.02341 |
| 5e | 0.00499 | 7.8144 | 0.02453 |
| 5f | 0.00529 | 7.8237 | 0.02198 |
| 5g | 0 | 7.8129 | 0.02886 |
| 8a | 0.00364 | 7.8133 | 0.02041 |
| 8b | 0.00259 | 7.8191 | 0.01997 |
| 8c | 0.00365 | 7.7989 | 0.02215 |
| 8d | 0.00209 | 7.8229 | 0.02199 |
| 8e | 0.00389 | 7.8328 | 0.02284 |
| 8f | 0.00365 | 7.8211 | 0.02517 |
| 8g | 0.00278 | 7.8116 | 0.02851 |

^{a}D₁ = Avg. nucleoph. react. index for atom N, D₂ = Max. atomic state energy for atom H, D₃ = Max. electroph. react. index for atom C.

Maximum atomic state energy for atom H is a semi-empirical descriptor with a negative coefficient sign in the attained QSAR model. The estimated results correlate with the low observed analgesic properties of compound 8c relative to 8e (descriptor values: 7.7989 and 7.8328; estimated % protection/inhibition: 42.5 and 87.5, respectively). The electron-electron repulsion and attraction energies for a given atomic species can be determined by equs. (5) and (6), respectively.

$$E \text{?} ( A ) = {\sum }_{B \neq A} {\sum }_{μ , v ∈ A} {\sum }_{λ , σ ∈ B} P \text{?} {P}_{λσ} 〈 μ v \vert λσ 〉 \qquad ( 5 )$$

$$E \text{?} ( AB ) = {\sum }_{B} {\sum }_{μ , v ∈ A} P \text{?} 〈 μ \vert \frac{Z \text{?}}{R \text{?}} \vert \text{?} 〉 \qquad ( 6 )$$

$$\text{?} \text{indicates text missing or illegible when filed}$$

where, A and B are two different atoms; P_{μν}, P_{λσ} are the density matrix elements over atomic basis (μνλσ): [symbol] μν|λσ [symbol] is the electron repulsion integral on atomic basis {μνλσ}; P_{μν} is the density matrix elements over atomic basis {μν}; Z_{B} is the charge of atomic nucleus B; R_{lR} is the distance between the electron and atomic nucleus B; and

$$〈 μ \vert \frac{Z \text{?}}{R \text{?}} \vert \text{?} 〉$$

$$\text{?} \text{indicates text missing or illegible when filed}$$

is the electron-nucleus attraction integral on atomic basis {Ξν}.

Again, maximum electrophilic reactivity index for atom C is a semi-empirical descriptor with negative coefficient value (−1.28007) in the 2D-QSAR model attained. The estimated values correlate with the observed anti-inflammatory properties of conjugate 5c relative to conjugate 8a (descriptor values: 0.02583 and 0.02041; estimated % protection/inhibition: 86.0 and 47.4, respectively). Fukui atomic electrophilic reactivity index is determined by equ. (7).

$${E}_{A} = {\sum }_{f ∈ A} {C}_{fLUMO}^{2} / ( {ε}_{LUMO} + 10 ) \qquad ( 7 )$$

where, ε_{LUMO} and C_{jLUMO} are the lowest unoccupied molecular orbital energy and coefficients, respectively.

The comparative values of observed and calculated analgesic properties support the results obtained using QSAR model (Table 11).

### Central Analgesic QSAR Model

CODESSA-Pro was employed for adjusting the statistically robust three-descriptor QSAR model (R²=0.967, R²cvOO=0.941, R²cvMO=0.949), using the homogeneous (non-diverse) bioactive conjugates having variable biological properties (observed % protection=0.2-66.5) (Tables 13-15 and FIG. 11).

TABLE 13
Descriptors of the QSAR model for the exemplary conjugates.

| ID | Coefficient | s | t | Descriptor |
|---|---|---|---|---|
| 0 | −4500.61 | 371.777 | −12.1057 | Intercept |
| D₁ | 402.343 | 32.617 | 12.335 | Max. resonance energy |
| | | | | for bond H—C |
| D₂ | 615.863 | 93.4621 | 6.589 | FNSA-3 Fractional PNSA |
| | | | | (PNSA-3/TMSA) |
| | | | | (MOPAC PC) |
| D₃ | −8834.15 | 716.006 | −12.3381 | Max. 1-electron react. |
| | | | | index for atom N |

N = 14,
n = 3,
R2 = 0.967,
R2cvOO = 0.941,
R2cvMO = 0.949,
F = 97.863,
s2 = 20.067
% Protection = −4500.61 + (402.343 × D1) + (615.863 × D2) − (8834.15 × D3)

TABLE 14
Observed and estimated central analgesic properties for
the exemplary conjugates according to the QSAR model

| Compound | Observed property | Estimated property | Error^{a} |
|---|---|---|---|
| 5a | 54.5 | 49.5 | 5.0 |
| 5b | 27.1 | 28.0 | −0.9 |
| 5c | 18 | 20.5 | −2.5 |
| 5d | 1.2 | −2.1 | 3.3 |
| 5e | 28 | 31.3 | −3.3 |
| 5f | 0.2 | 2.1 | −1.9 |
| 5g | 28.1 | 25.2 | 2.9 |
| 8a | 51.3 | 46.1 | 5.2 |
| 8b | 9.7 | 13.3 | −3.6 |
| 8c | 48.1 | 44.3 | 3.8 |
| 8d | 48.6 | 56.4 | −7.8 |
| 8e | 28.9 | 25.5 | 3.4 |
| 8f | 66.5 | 68.4 | −1.9 |
| 8g | 2.7 | 4.4 | −1.7 |

^{a}Error is the difference between the observed and estimated property.

TABLE 15
Molecular descriptor values of the QSAR
model for the exemplary conjugates

| | Descriptors^{a} | | |
|---|---|---|---|
| Compound | D₁ | D₂ | D₃ |
| 5a | 11.4069 | −0.03877 | 0.00176 |
| 5b | 11.4186 | −0.03964 | 0.00466 |
| 5c | 11.4263 | −0.04921 | 0.00519 |
| 5d | 11.3564 | −0.03786 | 0.00536 |
| 5e | 11.3569 | −0.04154 | 0.00134 |
| 5f | 11.358 | −0.04395 | 0.00453 |
| 5g | 11.3875 | −0.08934 | 0.0001 |
| 8a | 11.413 | −0.05235 | 0.00147 |
| 8b | 11.3319 | −0.05631 | 0.00121 |
| 8c | 11.3948 | −0.06407 | 0.00003 |
| 8d | 11.4089 | −0.05398 | 0 |
| 8e | 11.3236 | −0.04844 | 0 |
| 8f | 11.44 | −0.0549 | 0 |
| 8g | 11.3328 | −0.08776 | 0.00007 |

^{a}D₁ = Max. resonance energy for bond H—C, D₂ = FNSA-3 Fractional PNSA (PNSA-3/TMSA) (MOPAC PC), D₃ = Max. 1-electron react. index for atom N.

Maximum resonance energy for bond H—C is a semi-empirical descriptor positively participated in the QSAR model, which directly determines the % protection (property) of the tested conjugates with high coefficient value (coefficient: 402.343). The high descriptor value correlates with the potent analgesic activity of conjugates 8f and 5d (descriptor value: 11.44 and 11.3564; estimated value: 68.4 and −2.1 for conjugates 8f and 5d, respectively). Resonance energy between two atoms can be calculated by equ. (8).

$${E}_{R} ( AB ) = {\sum }_{μ ∈ A} {\sum }_{v ∈ B} {P}_{μ v} {β}_{μ v} \qquad ( 8 )$$

where, A and B are two different atomic species; and P_{μν} and β_{μν} are the density matrix elements and resonance integrals, respectively, over the atomic basis {μν}.

Fractional PNSA is a charge-related descriptor that also positively participated in the QSAR model with a high coefficient value (coefficient: 615.863). The estimated values correlate with the high observed property of conjugate 8f over conjugate 5g (descriptor value: −0.0549 and −0.08934; estimated value: 68.4 and 25.2, respectively). Fractional atomic charge for the weighted surface area (partially positive) can be calculated by equ. (9).

$$PPSA 3 = \frac{PPSA 3}{TMSA} \qquad ( 9 )$$

where, PFSA3 and TMSA are the total charge (partially positive) weighted molecular surface area and the total molecular surface area, respectively.

Maximum 1-electron reactivity index for atom N is a semi-empirical descriptor negatively participated in the QSAR model with the highest coefficient value among all the other descriptors (coefficient: −8834.15). The conjugate with high descriptor value indicates low biological activity, as shown in conjugates 5d and 8f (descriptor value: 0.00536 and 0, with estimated value of −2.1 and 68.4, respectively). Fukui atomic one-electron reactivity index can be calculated by equ. (10).

$${R}_{A} = {\sum }_{t ∈ A} {\sum }_{f ∈ A} {C}_{tHOMO} {C}_{fLUMO} / ( {ε}_{LUMO} + {ε}_{HOMO} ) \qquad ( 10 )$$

where, C_{lHOMO} and C_{jLUMO} rare the highest occupied and the lowest unoccupied molecular orbital coefficients, respectively.

The estimated biological properties based on the QSAR model are comparable to the observed central analgesic properties (Table 14).

### CONCLUSION

Exemplary ibuprofen and indomethacin conjugates (5 and 8) were synthesized using a molecular hybridization approach. The conjugates were evaluated for their anti-inflammatory and analgesic properties. Compounds 5a, 5b, 5d, and 5e showed potent anti-inflammatory properties comparable to their parent compounds ibuprofen and indomethacin. No ulcerogenic liability was shown by tested conjugates 5a, 5b, 5d, and 5e. These anti-inflammatory conjugates were evaluated in vitro as COX-1/COX-2 inhibitors, which showed considerable selectivity towards COX-2 compared with their parent compounds ibuprofen and indomethacin. The suppression effect of LPS-stimulated production of NO (and thus ROS) and cytokines IL-6, TNF-α, and iNOS in RAW264.7 cells demonstrated the anti-inflammatory properties of the ibuprofen conjugates. Molecular modeling supported the observed biological properties.

### REFERENCES

W. L. Smith, et al., J. Biol. Chem. 271, 33157-33160 (1996).
R. G. Kurumbail, et al., Nature 384, 644-648 (1996).
W. L. Smith, et al., Annu. Rev. Biochem. 69, 145-182 (2000).
J. A. Mitchell and T. D. Warner, Br. J. Pharmacol. 128, 1121-1132 (1999).
E. Ricciotti and G. A. FitzGerald, Arterioscler. Thromb. Vasc. Biol. 31, 986-1000 (2011).
D. Wang and R. N. DuBois, Ann. Rev. Med. 64, 131-144 (2013).
D. Wang and R. N. DuBois, Nat. Rev. Cancer 10, 181-193 (2010).
J.-B. Meric, et al., Crit. Rev. Oncol. Hematol. 59, 51-64 (2006).
P. Teismann, et al., Proc. Natl. Acad. Sci. USA 100, 5473-5478 (2003).
A. Sorokin. Curr. Pharm. Des. 10, 647-657 (2004).
C. Luong, et al., Nat. Struct. Mol. Biol. 3, 927-933 (1996).
J. M. Dogne, et al., J. Med. Chem. 48, 2251-2257 (2005).
S. Nalamachu and R. Wortmann, Med. 126, 92-97 (2014).
G. Varrassi, et al., Adv. Ther. 37, 61-82 (2020).
F. Naaz, et al., Bioorg. Chem. 81, 1-20 (2018).
K. Bozorov, et al., Bioorg. Med. Chem. 27, 3511-3531 (2019).
B. Zhang, Eur. J. Med. Chem. 168, 357-372 (2019).
D.-K. Kim, et al., Bioorg. Med. Chem. Lett. 14, 2401-2405 (2004).
M. Whiting, et al., J. Med. Chem. 49, 7697-7710 (2006).
Z.-Y. Cheng, et al., Bioorg. Med. Chem. 15, 1533-1538 (2007).
F. d. C, da Silva, et al., Eur. J. Med. Chem. 44, 373-383 (2009).
H. M. Faidallah, et al., Eur. J. Med. Chem. 143, 1524-1534 (2018).
I. A. Seliem, et al., Bioorg. Chem. 114, 105117 (2021).
S. S. Panda, et al., Future Med. Chem. 12, 1369-1386 (2020).
S. S. Panda. et al., Eur. J. Med. Chem. 196, 112293 (2020).
A. S. Girgis, et al., Eur. J. Med. Chem. 50, 1-8 (2012).
W. Wang, et al., Sci. Rep. 7, 44252 (2017).
Friesner R A, et al., J Med Chem. 2004; 47(7):1739-49.
Friesner R A, et al., J Med Chem. 2006; 49(21):6177-96.
Kurumbail R G, et al., Nature. 1996; 384(6610):644-8.
Sidhu R S, et al., Biochemistry. 2010; 49(33):7069-79.
Dvorakova, M, ACS Med. Chem. Lett. 2021; 12(4):610-616.
Rouzer, C. A. and Marnett, L. J., Chem. Rev. 2020; 120:7592-7641.
Lou, Y. and Zhu, J., Bioactive Carboxylic Compound Classes 2016; 221-236.
Blobaum, A. L. and Marnett, L. J., J. Biol. Chem. 2007; 282:16379-16390.
Genheden, S. and Ryde, U., Expert Opin. Drug Discov. 2015; 10:449-461.
Dong, L., et al., Biochemistry 2019; 58:3990-4002.
Baek, S. H., et al., Int. J. Mol. Sci. 22, 3659.
A. S. Girgis, et al., Org. Biomol. Chem. 13, 1741-1753 (2015).
Z. M. Nofal, et al., Eur. J. Med. Chem. 63, 14-21 (2013).
CODESSA PRO User's Manual (http://www.codessa-pro.com/manuals/manual.htm;
accessed on Jul. 30, 2022).
J. Li, et al., Beilstein J. Org. Chem. 9, 908-917 (2013).
K. K. Laali, et al., Chemistry Open 9, 822-834 (2020).
M. Korczak, et al., Sci. Rep. 12, 11676 (2022).
COX-1/2 (human) inhibitor screening assay kits (Fluorometric, Catalog #K547-100 and K548-100), BioVision Incorporated (155 S. Milpitas Blvd., Milpitas, CA 95035 USA, www.biovision.com).

I claim:

1. A compound having the structure of: 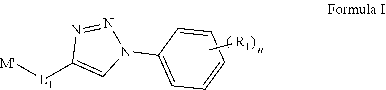
wherein:
(i) R₁ is a halide, an unsubstituted alkyl, a substituted alkyl, an alkoxy, a nitro, a carbonyl, a hydroxyl, an amino, an amido, a cyano, a thio, an unsubstituted alkenyl, a substituted alkenyl, an unsubstituted alkynyl, a substituted alkynyl, an unsubstituted alkylaryl, a substituted alkylaryl, an unsubstituted aryl, or a substituted aryl;
(ii) n is an integer from 0 to 5, from 0 to 4, from 0 to 3, or 1 or 2, such as 1;
(iii) L₁ is 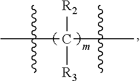
wherein m is an integer from 0 to 6,
R₂ and R₃ are independently H, an unsubstituted alkyl, or a substituted alkyl;
(iv) M′ is a parent drug moiety; and
(v) each substituent is independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a carbonyl, an alkoxy, a halogen, a hydroxyl, a phenoxy, a thiol, an alkylthio, a phenylthio, an arylthio, a cyano, an isocyano, a nitro, a carboxyl, an amino, an amido, an oxo, a silyl, a sulfinyl, a sulfonyl, a sulfonic acid, a phosphonium, a phosphanyl, a phosphoryl, or a phosphonyl.

2. The compound of claim 1, wherein M′ is 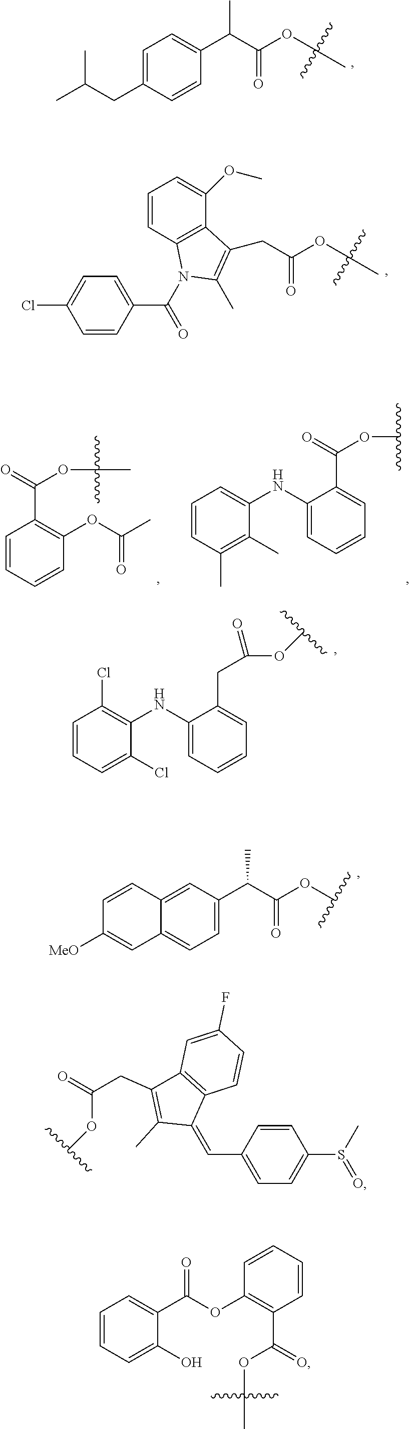 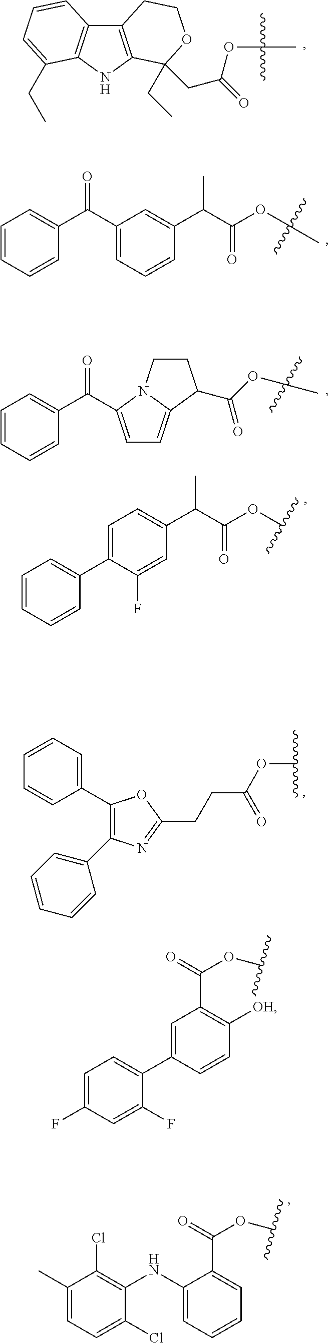 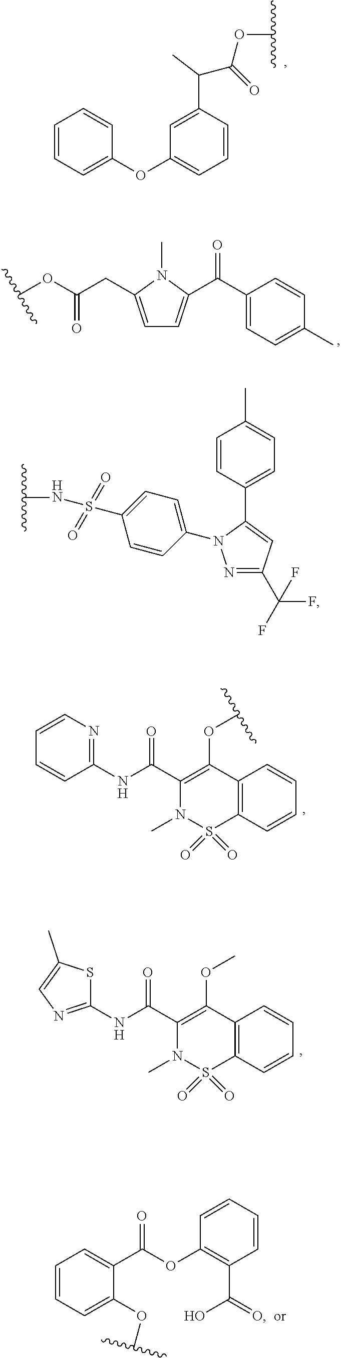 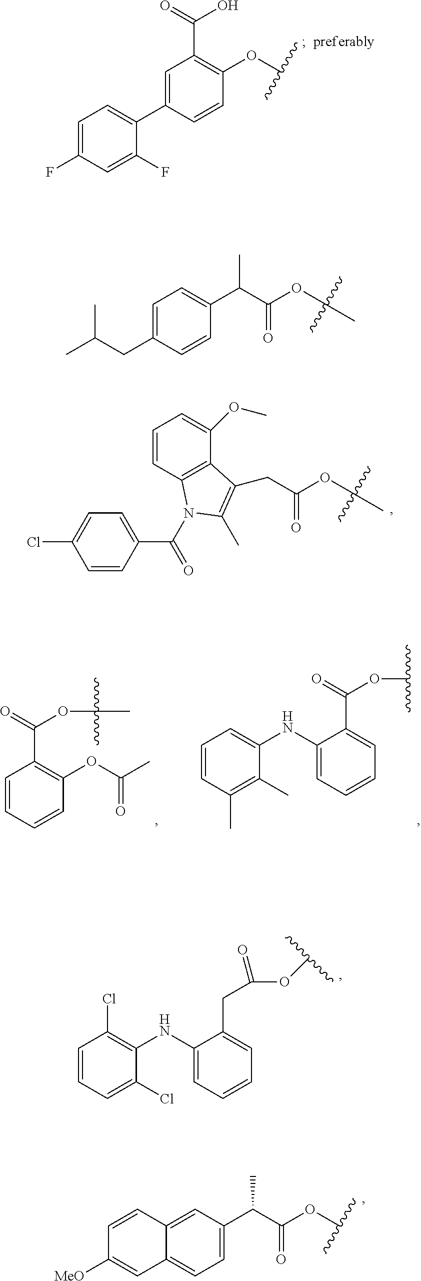 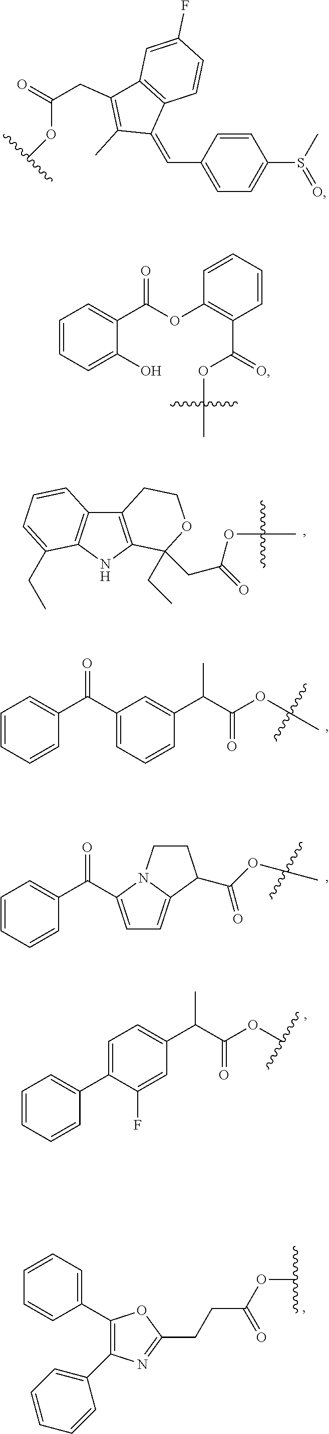 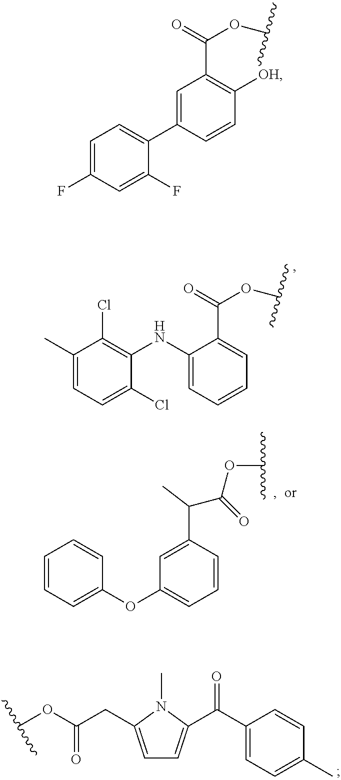 [SMILES: *OC(=O)c1cc(-c2ccc(F)cc2F)ccc1O ; *OC(=O)c1ccccc1Nc1c(Cl)ccc(C)c1Cl ; *OC(=O)C(C)c1cccc(Oc2ccccc2)c1 ; *OC(=O)Cc1ccc(C(=O)c2ccc(C)cc2)n1C]
more preferably or

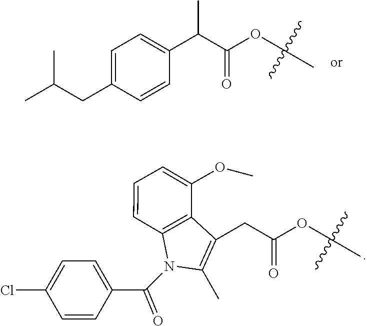 [SMILES: *OC(=O)C(C)c1ccc(CC(C)C)cc1 ; *OC(=O)Cc1c(C)n(C(=O)c2ccc(Cl)cc2)c2cccc(OC)c12]

3. The compound of claim 1 or 2, wherein m is an integer from 1 to 4, and R₂ and R₃ are independently H or an unsubstituted linear or branched C₁-C₆ alkyl, such as methyl, ethyl, 1-propanol, isopropanol, 1-butyl, isobutyl, or tertbutyl.

4. The compound of claim 3, wherein m is 1 or 2, and R₂ and R₃ are independently H or methyl.

5. The compound of any one of claims 1-4, wherein n is an integer from 1-3, such as 1.

6. The compound of any one of claims 1-5, wherein R₁ is a halide, an unsubstituted linear or branched C₁-C₆ alkyl, a nitro, or 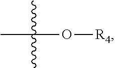
wherein R₄ is an unsubstituted linear or branched C₁-C₆ alkyl.

7. The compound of any one of claims 1-6, wherein n is 1 and R₁ is at the ortho- or para-position.

8. The compound of any one of claims 1-7, wherein the compound has the structure of:

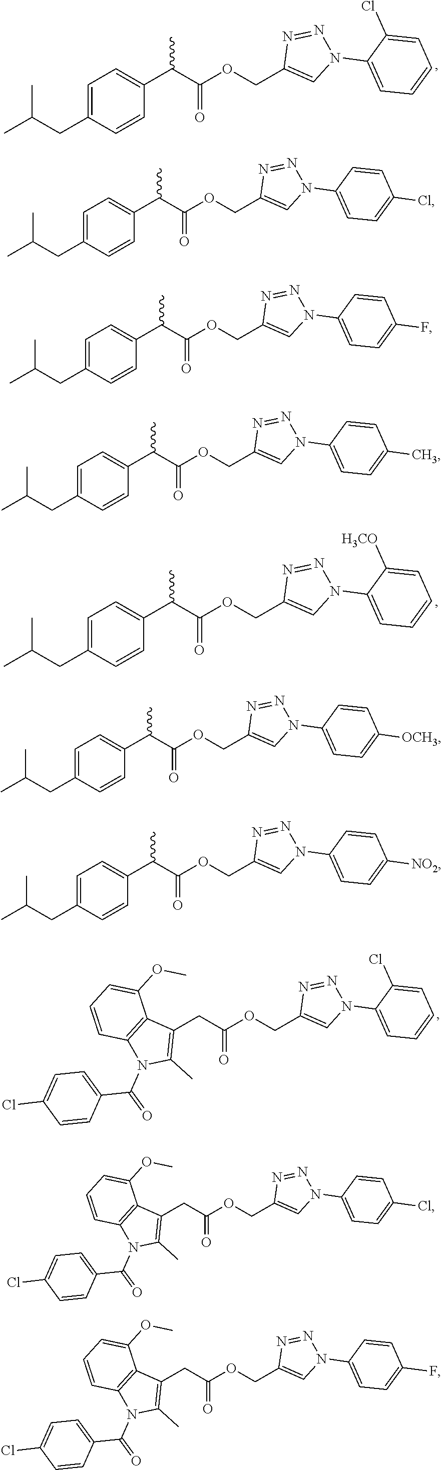

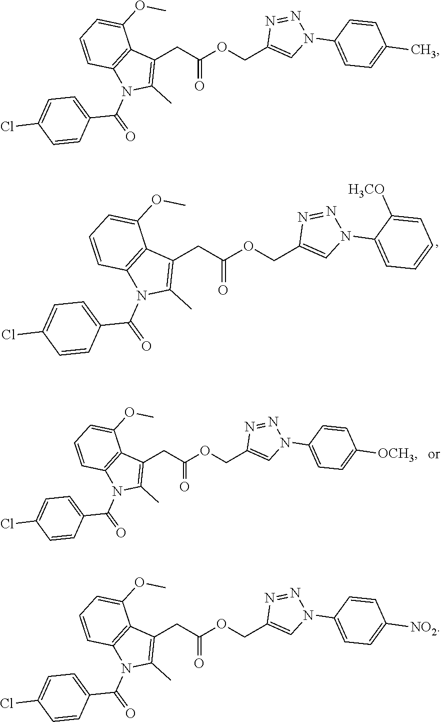 [SMILES: COc1cccc2c1c(CC(=O)OCc1cn(-c3ccc(C)cc3)nn1)c(C)n2C(=O)c1ccc(Cl)cc1 ; COc1ccccc1-n1cc(COC(=O)Cc2c(C)n(C(=O)c3ccc(Cl)cc3)c3cccc(OC)c23)nn1 ; COc1ccc(-n2cc(COC(=O)Cc3c(C)n(C(=O)c4ccc(Cl)cc4)c4cccc(OC)c34)nn2)cc1 ; COc1cccc2c1c(CC(=O)OCc1cn(-c3ccc([N+](=O)[O-])cc3)nn1)c(C)n2C(=O)c1ccc(Cl)cc1]

9. A pharmaceutical formulation comprising one or more compounds of any one of claims 1-8; and a pharmaceutically acceptable excipient,
wherein the one or more compounds are in an effective amount to prevent or treat an inflammatory disease or disorder in a subject.

10. The pharmaceutical formulation of claim 9, wherein the pharmaceutical formulation further comprises one or more active agents, and optionally wherein the one or more active agents is/are one or more anti-inflammatory agent.

11. The pharmaceutical formulation of claim 9 or 10, wherein the total amount of the one or more compounds in the pharmaceutical formulation is at least 0.01 wt %, at least 0.05 wt %, at least 0.1 wt %, in a range from 0.01 wt % to 50 wt %, from 0.05 wt % to 50 wt %, from 0.1 wt % to 50 wt %, from 0.01 wt % to 40 wt %, from 0.05 wt % to 40 wt %, from 0.1 wt % to 40 wt %, from 0.01 wt % to 30 wt %, from 0.05 wt % to 30 wt %, from 0.1 wt % to 30 wt %, from 0.01 wt % to 20 wt %, from 0.05 wt % to 20 wt %, from 0.1 wt % to 20 wt %, from 0.01 wt % to 10 wt %, from 0.05 wt % to 10 wt %, or from 0.1 wt % to 10 wt %.

12. A method of producing the compound of any one of claims 1-8 comprising:
(i) exposing a first reaction mixture to a microwave irradiation for a time period sufficient to form a product comprising the compound,
wherein the first reaction mixture comprises a first reactant, a second reactant, and a first solvent,
wherein the first reactant has the structure of: 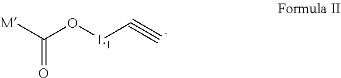
wherein the second reactant has the structure of: 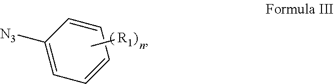
and
wherein:
(i) R₁ is a halide, an unsubstituted alkyl, a substituted alkyl, an alkoxy, a nitro, a carbonyl, a hydroxyl, an amino, an amido, a cyano, a thio, an unsubstituted alkenyl, a substituted alkenyl, an unsubstituted alkynyl, a substituted alkynyl, an unsubstituted alkylaryl, a substituted alkylaryl, an unsubstituted aryl, or a substituted aryl;
(ii) n is an integer from 0 to 5, from 0 to 4, from 0 to 3, or 1 or 2, such as 1;
(iii) L₁ is 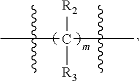
wherein m is an integer from 0 to 6,
R₂ and R₃ are independently H, an unsubstituted alkyl, or a substituted alkyl;
(iv) M′ is a parent drug moiety; and
(v) each substituent is independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a carbonyl, an alkoxy, a halogen, a hydroxyl, a phenoxy, a thiol, an alkylthio, a phenylthio, an arylthio, a cyano, an isocyano, a nitro, a carboxyl, an amino, an amido, an oxo, a silyl, a sulfinyl, a sulfonyl, a sulfonic acid, a phosphonium, a phosphanyl, a phosphoryl, or a phosphonyl.

13. The method of claim 12, wherein the microwave irradiation has an energy in a range from about 10 W to about 50 W, from about 10 W to about 40 W, from 10 W to about 30 W, or from 15 W to about 25 W, such as about 20 W.

14. The method of claim 12 or 13, wherein the first reaction mixture is maintained at a temperature in a range from about 30° C. to about 100° C., from about 40° C. to about 90° C., or from about 50° C. to about 80° C., such as about 70° C., for a time period in a range from about 30 mins to about 5 hours, from about 1 hour to about 4 hours, or from about 1 hour to about 3 hours, such as about 2 hours.

15. The method of any one of claims 12-14, wherein the first solvent is water, an alcohol, or a mixture thereof, such as a mixture of 1-butanol and water.

16. The method of any one of claims 12-15, wherein the reaction mixture further comprises a reducing agent and/or a first catalyst, and optionally wherein the reducing agent is sodium iso-ascorbate and/or copper thiophene carboxylate and the first catalyst is CuSO₄.

17. The method of any one of claims 12-16, wherein the reaction mixture is under stirring during step (i).

18. The method of any one of claims 12-17, wherein the method further comprises:
(a) maintaining a second reaction mixture at a suitable temperature for a time period sufficient to produce the first reactant,
wherein step (a) is performed prior to step (i),
wherein the second reaction mixture comprises a first starting reactant, a second starting reactant, and a second solvent,
wherein the first starting reactant has the structure of: 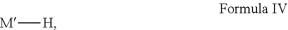
wherein the second starting reactant has the structure of: 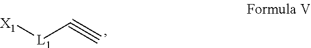
and
wherein:
(i) M′ and L₁ are as defined above in claim 12; and
(ii) X₁ is a halide, such as fluoride, bromide, or chloride.

19. The method of claim 18, wherein step (a) was maintained at room temperature (20° C. to 25° C. at 1 atm) for a time period in a range from 12 hour to 36 hours.

20. The method of claim 19, wherein prior to step (a), the second reaction mixture is heated from about 0° C. to the room temperature.

21. The method of any one of claims 18-20, wherein the second solvent is tetrahydrofuran, dichloromethane, dimethyl sulfoxide, dimethyl formamide, ethyl acetate, ethyl lactate, acetone, 1-butanol, 1-propanol, 2-propanol, ethanol, isopropyl acetate, methanol, methyl ethyl ketone, t-butanol, 2-methyl tetrahydrofuran, acetonitrile, or toluene, or a combination thereof.

22. The method of any one of claims 18-21, wherein the second reaction mixture further comprises a second catalyst, and optionally wherein the second catalyst is Cs₂CO₃, potassium carbonate, triethylamine, sodium bicarbonate, tetrabutylammonium fluoride, or potassium hydroxide, or combinations thereof.

23. The method of any one of claims 12-22, further comprising purifying the product containing the compound subsequent to step (i).

24. The method of any one of claims 12-23, wherein the compound has a yield of at least 60%, at least 65%, at least 70%, or at least 75%.

25. A method for preventing or treating an inflammatory disease or disorder in a subject in need thereof comprising
(i) administering to the subject the pharmaceutical formulation of any one of claims 9-11, wherein step (i) occurs one or more times.

26. The method of claim 25, wherein the method comprises only a single administration of the pharmaceutical formulation, wherein the pharmaceutical formulation comprises an effective amount of the compounds to change one or more clinical and/or biochemical measurements associated with the inflammatory disease or disorder of the subject (such as reduce or relief inflammation associated swelling and/or reduce or relief pain), compared to the subject before administered with the pharmaceutical formulation.

27. The method of claim 25, wherein the method comprises more than one step of administering to the subject the pharmaceutical formulation, wherein following all of the administration steps, an effective amount of the compounds to change one or more clinical and/or biochemical measurements associated with the inflammatory disease or disorder of the subject (such as reduce or relief inflammation associated swelling and/or reduce or relief pain), compared to the subject before administered with the pharmaceutical formulation, is administered to the subject.

28. The method of claim 26 or 27, wherein the effective amount of the compound is effective to reduce or relief inflammation associated swelling, as indicated by reduction of the edema thickness of a body part of the subject by at least 10%, at least 20%, at least 30%, at least 50%, at least 60%, at least 70%, at least 80%, or at least 90% within about 24 hours, about 12 hours, about 10 hours, about 5 hours, about 4 hours, about 3 hours, about 2 hours, or about 1 hour following the single administration or all of the administrations of the pharmaceutical formulation.

29. The method of any one of claims 26-28, wherein the effective amount of the compound is effective to reduce or relief pain associated with the inflammatory disease or disorder.

30. The method of any one of claims 26-29, wherein following the administration or all of the administrations of the pharmaceutical formulation, the subject does not develop an ulcer or erosion in the gastrointestinal system.

31. The method of any one of claims 25-30, wherein in step (i), the pharmaceutical formulation is administered by oral administration, intramuscular administration, intravenous administration, intraperitoneal administration, or subcutaneous administration, or a combination thereof.

32. The method of any one of claims 25-31, wherein during step (i), the dosage of the compounds in the pharmaceutical formulation is from about 0.1 mg to about 100 mg, from about 0.5 mg to about 50 mg, from about 1 mg to about 100 mg, from about 2 mg to about 100 mg, from about 2 mg to about 50 mg, from about 2 mg to about 25 mg, or from about 5 mg to about 20 mg per kg of the subject, such as about 10 mg per kg of the subject.

33. A method for treating macrophages in a subject in need thereof comprising:
(i) administering to the subject the pharmaceutical formulation of any one of claims 9-11, wherein step (i) occurs one or more times.

34. The method of claim 33, wherein the method comprises only a single administration of the pharmaceutical formulation, wherein the pharmaceutical formulation comprises an effective amount of the compounds to reduce production of nitric oxide by macrophages and/or reduce release of one or more inflammatory cytokines from the macrophages, compared with the subject before administered with the pharmaceutical formulation.

35. The method of claim 33, wherein the method comprises more than one step of administering to the subject the pharmaceutical formulation, wherein following all of the administration steps, an effective amount of the compounds to reduce production of nitric oxide by macrophages and/or reduce release of one or more inflammatory cytokines from the macrophages is administered to the subject, compared with the subject before administered with the pharmaceutical formulation.

36. The method of claim 34 or 35, wherein the reduction of the release of the one or more inflammatory cytokines is indicated by a decrease in the mRNA level associated with the inflammatory cytokine or each of the inflammatory cytokines.

37. The method of any one of claims 34-36, wherein the inflammatory cytokine is or each of the inflammatory cytokines independently is IL-6, TNF-α, or iNOS.
